(19) United States
(12) Patent Application Publication
(10) Pub. No.: US 2017/0334991 A1
(43) Pub. Date: Nov. 23, 2017

(54) ANTI-CLDN CHIMERIC ANTIGEN RECEPTORS AND METHODS OF USE

(71) Applicant: ABBVIE STEMCENTRX LLC, NORTH CHICAGO, IL (US)

(72) Inventors: PAUL ANTHONY ESCARPE, SAN BRUNO, CA (US); SCOTT J. DYLLA, EMERALD HILLS, CA (US); ROBERT A. STULL, ALAMEDA, CA (US); DAVID LIU, SAN FRANCISCO, CA (US)

(73) Assignee: ABBVIE STEMCENTRX LLC, NORTH CHICAGO, IL (US)

(21) Appl. No.: 15/524,675

(22) PCT Filed: Nov. 4, 2015
(86) PCT No.: PCT/US15/59106

§ 371 Date: May 5, 2017

Related U.S. Application Data
(60) Provisional application No. 62/157,928, filed on May 6, 2015.
(60) Provisional application No. 62/247,108, filed on Oct. 27, 2015.

(51) Int. Cl. C07K 16/28

(52) U.S. Cl. CPC C07K 16/28; C07K 2319/03; C07K 2317/14; C07K 2317/24; C07K 2317/622

(57) ABSTRACT

Provided herein are novel anti-CLDN chimeric antigen receptors and methods of using the same to treat proliferative disorders.

### CROSS REFERENCED APPLICATIONS

This application claims the benefit of Patent Cooperation Treaty (PCT) Application No. PCT/US2014/064165 filed on 5 Nov. 2014, U.S. Provisional Application No. 62/157,928 filed on 6 May 2015 and U.S. Provisional Application No. 62/247,108 filed on 27 Oct. 2015, each of which is incorporated herein by reference in its entirety.

### SEQUENCE LISTING

This application contains a sequence listing which has been submitted in ASCII format via EFS-Web and is hereby incorporated by reference in its entirety. Said ASCII copy, created on Nov. 3, 2015 is named sc2703pct_S69697_1270WO_SEQL_110315.txt and is 247,510 bytes in size.

### FIELD OF THE INVENTION

The present invention generally relates to adoptive immunotherapy comprising the use of novel chimeric antigen receptors incorporating a claudin (CLDN) binding domain. In preferred embodiments the disclosed chimeric antigen receptors are useful for the treatment or prophylaxis of proliferative disorders and any recurrence or metastasis thereof.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rates for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

In a broad aspect the present invention provides novel chimeric antigen receptors (CARs) comprising a CLDN binding domain that specifically binds to at least one protein of the CLDN family (CLDN CARs). In selected embodiments the CLDN CARs of the invention bind specifically to CLDN6 or specifically to CLDN6 and CLDN9. In yet other embodiments the antibodies of the invention bind to CLDN6 and CLDN9 with substantially the same apparent binding affinity. In other embodiments the disclosed CARs may cross-react with CLDN4. Yet in other preferred embodiments the CLDN target protein(s) are expressed on tumor initiating cells.

Through genetic modification (e.g., transduction) the CLDN CAR are expressed on cytotoxic lymphocytes (preferably autologous) to provide CLDN sensitive lymphocytes that are used to target and kill CLDN positive tumor cells. As will be discussed extensively herein the CARs of the instant invention generally comprise an extracellular domain comprising a CLDN binding domain, a transmembrane domain and an intracellular signaling domain that activates certain lymphocytes and generates an immune response directed to CLDN positive tumor cells (i.e., those that are CLDN6+ and/or CLDN9+ and/or CLDN4+). Selected embodiments of the invention comprise immunoactive host cells expressing the disclosed CARs and various polynucleotide sequences and vectors encoding the CLDN CARs of the invention. Yet other aspects include methods of enhancing T lymphocyte or natural killer (NK) cell activity in an individual and treating an individual suffering from cancer by introducing into the individual host cells expressing CLDN CAR molecules. Such aspects specifically include the treatment of lung cancer (e.g., small cell lung cancer), melanoma, breast cancer, prostate cancer, colon cancer, renal cell carcinoma, ovarian cancer, neuroblastoma, rhabdomyosarcoma, leukemia and lymphoma.

As discussed in more detail below, the term “antibody” as used herein shall be held to mean intact antibodies (e.g., IgG or IgM) as well as any immunoreactive fragments (e.g., Fab fragments) or immunoreactive constructs or derivatives thereof (e.g., scFv). In certain embodiments the CLDN binding domains (and CLDN CARs) of the instant invention will comprise scFv constructs and, in preferred embodiments, will comprise scFv constructs that compete for binding with antibodies comprising heavy and light chain variable regions as disclosed herein. In other preferred embodiments the CLDN binding domains (and CLDN CARs) of the invention will comprise scFv constructs comprising heavy and light chain variable regions disclosed herein or fragments thereof. As such, for the purposes of the instant disclosure the term “antibody” shall be used generally and will expressly be held to include immunoreactive fragments, constructs or derivatives thereof unless otherwise contextually dictated.

In particular aspects of the invention the CAR binding domain binds to at least one member of the CLDN family (e.g., CLDN4, CLDN6 and/or CLDN9) and will be derived from, comprise or compete for binding with an antibody or antibody fragment comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59. In particularly preferred embodiments the CLDN binding domain will comprise a scFv construct comprising the aforementioned VL and VH sequences or fragments thereof. In some aspects of the invention the CAR binding domain comprises a chimeric, CDR grafted, humanized or human antibody or an immunoreactive fragment thereof. In other aspects of the invention the CAR binding domain comprising the aforementioned sequences is an internalizing antibody.

In yet other embodiments the CAR binding domain binds specifically to CLND6; or binds specifically to CLDN6 and CLDN9 and competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59.

Yet other preferred CARs of the instant invention will comprise CDR grafted or humanized CAR binding domains comprising one or more heavy (CDRH1, CDRH2, CDRH3) or light (CDRL1, CDRL2, CDRL3) chain CDRs as set forth in FIGS. 3A or 3B wherein the CDRs are derived as per Kabat et al.

In a particular embodiment the invention comprises a binding domain that binds to at least one protein of the CLDN family and competes for binding with an antibody comprising three variable light chain CDRs (CDRL) as set forth in SEQ ID NO: 61; and three variable heavy chain CDRs (CDRH) as set forth in SEQ ID NO: 63; or three CDRL as set forth in SEQ ID NO: 65 and three CDRH as set forth in SEQ ID NO: 67; or three CDRL as set forth in SEQ ID NO: 69 and three CDRH as set forth in SEQ ID NO: 71; three CDRL as set forth in SEQ ID NO: 73 and three CDRH as set forth in SEQ ID NO: 87.

In a further embodiment the invention comprises a humanized or CDR grafted binding domain that binds to at least one protein of the CLDN family and competes for binding with an antibody comprising a VH and VL, wherein the VL has three CDRL comprising a CDRL1 of SEQ ID NO: 151, a CDRL2 of SEQ ID NO: 152 and a CDRL3 of SEQ ID NO: 153; or a VL having three CDRLs comprising a CDRL1 of SEQ ID NO: 157, a CDRL2 of SEQ ID NO: 158 and a CDRL3 of SEQ ID NO: 159; or a VL having three CDRLs comprising a CDRL1 of SEQ ID NO: 163, a CDRL2 of SEQ ID NO: 164 and a CDRL3 of SEQ ID NO: 165; or a VL having three CDRLs comprising a CDRL1 of SEQ ID NO: 169, a CDRL2 of SEQ ID NO: 170 and a CDRL3 of SEQ ID NO: 171.

In a further embodiment the invention comprises a humanized or CDR grafted binding domain that binds to at least one protein of the CLDN family and competes for binding with an antibody comprising a VL and a VH, wherein the VH has three CDRs (CDRH) comprising a CDRH1 of SEQ ID NO: 154, a CDRH2 of SEQ ID NO: 155 and a CDRH3 of SEQ ID NO: 156; or the VH has three CDRHs comprising a CDRH1 of SEQ ID NO: 160, a CDRH2 of SEQ ID NO: 161 and a CDRH3 of SEQ ID NO: 162; or the VH has three CDRHs comprising a CDRH1 of SEQ ID NO: 166, a CDRH2 of SEQ ID NO: 167 and a CDRH3 of SEQ ID NO: 168; or the VH has three CDRHs comprising a CDRH1 of SEQ ID NO: 172, a CDRH2 of SEQ ID NO: 173 and a CDRH3 of SEQ ID NO: 174; or the VH has three CDRHs comprising a CDRH1 of SEQ ID NO: 172, a CDRH2 of SEQ ID NO: 176 and a CDRH3 of SEQ ID NO: 174.

In a further embodiment the invention comprises a humanized or CDR grafted binding domain that binds to at least one protein of the CLDN family and competes for binding with an antibody comprising a VL and VH wherein the VL has three CDRLs comprising a CDRL1 of SEQ ID NO: 151, a CDRL2 of SEQ ID NO: 152 and a CDRL3 of SEQ ID NO: 153 and the VH has three CDRHs comprising a CDRH1 of SEQ ID NO: 154, a CDRH2 of SEQ ID NO: 155 and a CDRH3 of SEQ ID NO: 156; or an antibody comprising a VL and VH wherein the VL has three CDRLs comprising a CDRL1 of SEQ ID NO: 157, a CDRL2 of SEQ ID NO: 158 and a CDRL3 of SEQ ID NO: 159 and the VH has three CDRHs comprising a CDRH1 of SEQ ID NO: 160, a CDRH2 of SEQ ID NO: 161 and a CDRH3 of SEQ ID NO: 162; or an antibody comprising a VL and VH wherein the VL has three CDRLs comprising a CDRL1 of SEQ ID NO: 163, a CDRL2 of SEQ ID NO: 164 and a CDRL3 of SEQ ID NO: 165 and the VH has three CDRHs comprising a CDRH1 of SEQ ID NO: 166, a CDRH2 of SEQ ID NO: 167 and a CDRH3 of SEQ ID NO: 168; or an antibody comprising a VL and VH wherein the VL has three CDRLs comprising a CDRL1 of SEQ ID NO: 169, a CDRL2 of SEQ ID NO: 170 and a CDRL3 of SEQ ID NO: 171 and the VH has three CDRHs comprising a CDRH1 of SEQ ID NO: 172, a CDRH2 of SEQ ID NO: 173 and a CDRH3 of SEQ ID NO: 174; or an antibody comprising a VL and VH wherein the VL has three CDRLs comprising a CDRL1 of SEQ ID NO: 169, a CDRL2 of SEQ ID NO: 170 and a CDRL3 of SEQ ID NO: 171 and the VH has three CDRHs comprising a CDRH1 of SEQ ID NO: 172, a CDRH2 of SEQ ID NO: 176 and a CDRH3 of SEQ ID NO: 174.

In particularly preferred embodiments the CAR binding domain will compete with an antibody comprising a VL of SEQ ID NO: 69 and a VH of SEQ ID NO: 71. In other embodiments the CAR construct will comprise a binding domain comprising a VL of SEQ ID NO: 69 and a VH of SEQ ID NO: 71. Preferably the binding domain will comprise a scFv.

In other particularly preferred embodiments the CAR binding domain will compete with an antibody comprising a VL of SEQ ID NO: 73 and a VH of SEQ ID NO: 87. In other embodiments the CAR construct will comprise a binding domain comprising a VL of SEQ ID NO: 73 and a VH of SEQ ID NO: 87. Preferably the binding domain will comprise a scFv. In certain preferred embodiments each of the aforementioned chimeric antigen receptors comprises a humanized or CDR grafted binding domain.

The term “compete” or “competing antibody” when used in the context of the disclosed binding domains means binding competition between antibodies as determined by an assay in which a reference antibody or immunologically functional fragment substantially prevents or inhibits (e.g., greater than 30%, 35%, 40%, 45%, 50%, 55%, 60%, 65%, 70%, 75%, 80%, 85% or 90%.) specific binding of a test antibody to a common antigen. Compatible methods for determining such competition comprise art known techniques such as, for example, bio-layer interferometry, surface plasmon resonance, flow cytometry, competitive ELISA, etc.

In certain embodiments the invention is directed to a nucleic acid encoding a CAR comprising the heavy or light chain amino acid sequence of any one of the anti-CLDN binding domains disclosed herein. In this regard nucleic acid sequences encoding such exemplary humanized or CDR grafted heavy and light chain variable regions are set forth in attached FIG. 3C and sequence listing. In preferred embodiments the nucleic acid encoding the binding domain or CAR are incorporated in a plasmid or vector. In yet other embodiments the vector will comprise a viral vector.

In other embodiments the present invention provides methods of treating cancer such as pancreatic cancer, ovarian cancer, colorectal cancer, small cell and non-small cell lung cancer, and gastric cancer, comprising administering a pharmaceutical composition comprising a host cell expressing an anti-CLDN CAR as disclosed herein.

In some embodiments, the invention provides methods of treating cancer comprising administering a pharmaceutical composition comprising a host cell expressing an anti-CLDN CAR as disclosed herein and further comprising administering to the subject at least one additional therapeutic moiety. In preferred embodiments the host cell will comprise a sensitized lymphocyte.

The present invention also provides a method of reducing tumor initiating cells in a tumor cell population, wherein the method comprises contacting a tumor cell population comprising tumor initiating cells and tumor cells other than tumor initiating cells, with a host cell expressing an anti-CLDN CAR; whereby the frequency of tumor initiating cells is reduced.

In yet other preferred embodiments the present invention also provides kits or devices and associated methods that are useful in the treatment of CLDN associated disorders such as cancer. To this end the present invention preferably provides an article of manufacture useful for generating CLDN sensitized lymphocytes for treating CLDN associated disorders comprising, for example, a receptacle containing vectors (e.g., viral vectors) encoding the disclosed CARs and instructional materials for generating CLDN sensitized lymphocytes. In selected embodiments the kits will comprise additional reagents and receptacles to effectively transduce the lymphocytes. In certain selected embodiments the kits will be used to transduce autologous lymphocytes obtained from the patient to be treated. In other selected embodiments such kits comprise allogeneic CLDN sensitized lymphocytes that may be directly administered to the patient to generate the desired immune response.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1 shows the relative mRNA expression levels of CDLN4, CLDN6, and CLDN9 determined by whole transcriptome (SOLiD) sequencing in selected patient-derived xenograft (PDX) tumors wherein tumor types are denoted according the abbreviations listed in Table 3 below;

FIGS. 2A-2C illustrate the relationship between CLDN family members wherein FIG. 2A is a dendrogram showing the relative degree of similarity between the 30 CLDN proteins encoded by the 23 human CLDN genes, FIG. 2B is a tabular representation of the percent identity of amino acid residues in extracellular domain (ECD) 1 or ECD2 in CLDN4, CLDN6 and CLDN9 and FIG. 2C is a tabular representation of the percent identity of amino acid residues in the ECD1 and ECD2 loops amongst the 16 proteins comprising the set of human, rat, mouse and cynomolgus monkey orthologs of CLDN4, CLDN6 and CLDN9;

FIGS. 3A-3C provide, in a tabular form, light chain (FIG. 3A) and heavy chain (FIG. 3B) contiguous variable region amino acid sequences of exemplary mouse and humanized anti-CLDN antibodies (SEQ ID NOS: 21-75, odd numbers) and variants of hSC27.22, hSC27.108 and hSC27.204 while FIG. 3C shows the nucleic acid sequences of the same light and heavy chain variable regions of such exemplary mouse and humanized anti-CLDN antibodies (SEQ ID NOS: 20-74, even numbers) along with variants of antibodies hSC27.22, hSC27.108 and hSC27.204;

FIGS. 4A-4C demonstrate the cross reactivity of certain binding domains of the invention where FIG. 4A shows the ability of anti-CLDN binding domains SC27.1 and SC27.22 to bind HEK-293T cells overexpressing human CLDN4, CLDN6 and CLDN9, FIG. 4B shows the ability of anti-CLDN antibodies to bind HEK-293T cells overexpressing CLDN4, CLDN6 and CLDN9 and FIG. 4C graphically illustrates the apparent binding affinity of an exemplary anti-CLDN antibody for CLDN6 and CLDN9 as determined by a titration of the amount of antibody versus a fixed number of cells expressing the antigen of interest;

FIG. 5 provides a schematic representation of an exemplary CLDN CAR construct compatible with the teachings herein;

FIGS. 6A-6D depict nucleic acid (FIGS. 6A and 6C) and amino acid (FIGS. 6B and 6D) sequences of two novel chimeric antigen receptors SCT1-SC27.108 and SCT1-SC27.204v2 of the instant invention;

FIG. 7 provides a schematic representation illustrating a process for producing CLDN sensitized lymphocytes and their subsequent use to generate an immune response directed to CLDN positive tumor cells;

FIGS. 8A and 8B demonstrate the expression of an exemplary CLDN CAR on transduced Jurkat cells (FIG. 8A) and the expression of hCLDN on engineered HEK-293T control cells (FIG. 8B) each as measured using flow cytometry;

FIGS. 9A and 9B depict the ratios of CLDN CAR Jurkat cells to CLDN expressing control cells used to gauge the activity of exemplary CLDN CAR cells (FIG. 9A) and the induction of an immune response in SCT1-h27.108 or SCT1-h27.204v2 transduced Jurkat cells as measured by IL-2 production (FIG. 9B);

FIG. 10 demonstrates that human lymphocytes from two individuals may be engineered to effectively express anti-CLDN CARs (SCT1-h27.108 or SCT1-h27.204v2) in accordance with the teachings herein;

FIGS. 11A and 11B provide CLDN surface expression profiles for a 293T cell line engineered to express CLDN6 (FIG. 11A) and an ovarian cancer patient derived xenograft (“PDX”) cell line (FIG. 11B) as evidenced by flow cytometry;

FIGS. 12A and 12B demonstrate the ability of CLDN sensitized lymphocytes comprising host cells from two individuals to provoke an immune response (as measured by the induction of INFγ) when exposed to engineered 293T cells (FIG. 12A) or PDX tumor cells (FIG. 12B);

FIGS. 13A and 13B demonstrate the ability of CLDN sensitized lymphocytes comprising host cells from two individuals to provoke an immune response (as measured by the induction of TNFα) when exposed to engineered 293T cells (FIG. 13A) or PDX tumor cells (FIG. 13B); and

FIGS. 14A and 14B show the ability of CLDN sensitized lymphocytes comprising host cells from two individuals to eliminate engineered 293T cells (FIG. 14A) or PDX tumor cells (FIG. 14B) upon exposure.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

Recent advances in adoptive transfer immunotherapy have provided a promising approach for the treatment of various neoplasia and a chance to improve patient experiences, particularly with regard to solid tumors. In this regard the present invention is directed to the use of novel chimeric antigen receptors (“CARs”) comprising an extracellular binding or targeting domain that associates or reacts with at least one claudin family member (“CLDN”). Preferably the CARs will immunospecifically associate with at least one of CLDN6, CLDN4 and/or CLDN9. As will be discussed extensively herein, CLDN (e.g., CLDN6) is a particularly effective tumor marker that is expressed on a number of different cancers and, significantly, has been found to be associated with cancer stem cells. Thus, when the anti-CLDN binding domains of the instant invention are incorporated in a chimeric antigen receptor expressed on lymphocytes, the resulting “CLDN sensitized lymphocytes” (e.g., natural killer cells or T cells that immunospecifically recognize CLDN) are able to effectively mount an immune response directed to aberrant CLDN positive cells including cancer stem cells. This ability to effectively eliminate tumorigenic “seed” cells is often critical in reducing the possibility of tumor recurrence or metastasis. To this end it will be appreciated that the anti-CLDN CAR T cells of the instant invention may be used in combination with other therapeutic agents (including anti-CLDN antibody drug conjugates) or as part of a maintenance regimen following standard of care treatments.

More generally a chimeric antigen receptor is an artificially constructed hybrid protein or polypeptide containing or comprising an antigen binding domain of an antibody linked to a signaling domain (e.g., T-cell signaling or T-cell activation domains). CARs have the ability to redirect such sensitized lymphocyte (e.g., T-cells) specificity and reactivity toward CLDN positive target cells in a non-MHC-restricted manner, exploiting the antigen-binding properties of monoclonal antibodies. The non-MHC-restricted antigen recognition gives T-cells expressing CARs the ability to recognize a CLDN independent of antigen processing, thus bypassing a major mechanism of tumor escape. Moreover, when expressed in T-cells, CARs advantageously do not dimerize with endogenous T cell receptor (TCR) alpha and beta chains, which may occur when applying TCR engineering techniques.

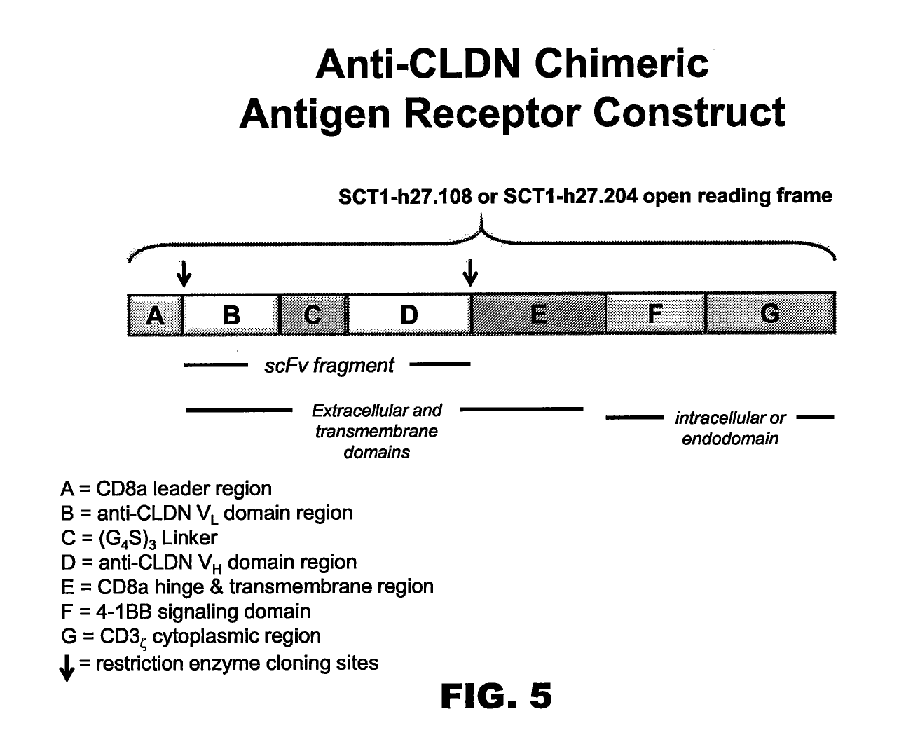

Accordingly, the present invention is generally directed to chimeric antigen receptors comprising a CLDN binding domain that immunospecifically associates with CLDN on target cells and stimulates an immune response. In preferred embodiments the CLDN binding domain of the CAR may comprise a scFv derived from the heavy and light chain antibody variable regions disclosed herein. More specifically an “anti-CLDN CAR” or simply “CLDN CAR” of the instant invention shall comprise a chimeric protein incorporating an extracellular CLDN binding domain, a transmembrane domain and an intracellular signaling domain (see FIG. 5). Typically a nucleotide sequence encoding the desired CLDN CAR will be synthesized or engineered and inserted into an expression vector or system (e.g. lentiviral, retroviral, etc.). In preferred embodiments lymphocytes, including T-lymphocytes and natural killer cells (“NK cells”), obtained from a patient or donor are then exposed to (e.g., transduced) the selected CLDN CAR vector to provide engineered lymphocytes that express the CAR protein with the extracellular CLDN binding domain (i.e., “CLDN sensitized lymphocytes”). In other embodiments allogeneic cells may be engineered to express the disclosed CARs to provide CLDN sensitized lymphocytes. Following optional expansion, these CLDN sensitized lymphocytes may be infused into a patient to mount an immunospecific response to CLDN positive tumor cells (see generally FIG. 7). In this regard the CLDN sensitized lymphocytes will be activated upon contacting a target cell expressing a CLDN determinant. To activate the sensitized lymphocytes (e.g., T cells and NK cells) means to induce a change in theft biologic state by which the cells express activation markers, produce cytokines, proliferate and/or become cytotoxic to target cells. All these changes can be produced by primary stimulatory signals, preferably along with co-stimulatory signals that amplify the magnitude of the primary signals and suppress cell death following initial stimulation resulting in a more durable activation state and thus a higher cytotoxic capacity.

It will further be appreciated that, besides the CLDN binding domain, CARs of the invention will comprise an intracellular or cytoplasmic domain that initiates a primary cytoplasmic signaling sequence (e.g., a sequence for initiating antigen-dependent primary activation via a T-cell receptor complex). Compatible intracellular domains may, for example, be derived from CD3ζ, FcRγ, FcRβ, CD3γ, CD3δ, CD3ε, CD5, CD22, CD79a, CD79b, and CD66d. In other preferred embodiments, the CARs of the invention will comprise an intracellular domain that initiates a secondary or co-stimulating signal. Compatible co-stimulatory domains may comprise, for example, intracellular domains derived from CD2, CD4, CD5, CD8α, CD8β, CD28, CD134, CD137, ICOS, CD154, 4-1BB and glucocorticoid-induced tumor necrosis factor receptor (see U.S.P.N. US/2014/0242701). Additionally, in preferred embodiments the disclosed CARs will comprise a transmembrane (and optionally a spacer) domain interposed between the extracellular CLDN binding domain and the intracellular signaling domain. As discussed in more detail below the transmembrane domain may comprise, for example, part of an antibody constant (Fc) region, human CD8a or artificially produced spacers known in the art. Essentially, any amino acid sequence that anchors the CAR in the cell membrane and allows for effective association of the CLDN binding domain and transmission of appropriate signaling from the intracellular domain is compatible with the invention.

With respect to the novel CLDN CARs of the invention it will be appreciated that the selection of CLDN as the tumor target is integral in generating an effective immune response. More specifically it has been found that CLDN phenotypic determinants are clinically associated with various proliferative disorders, including several types of cancer, and that CLDN proteins and variants or isoforms thereof provide useful tumor markers which may be exploited in the treatment of related diseases. In this regard the present invention provides a number of chimeric antigen receptors that comprise an anti-CLDN binding domain in addition to any signaling component. As discussed in more detail below, the disclosed CLDN CARs are particularly effective at eliminating tumorigenic cells and therefore useful for the treatment and prophylaxis of certain proliferative disorders or the progression or recurrence thereof.

Moreover, as set forth in the instant application it has been found that CLDN markers or determinants such as cell surface CLDN proteins are therapeutically associated with cancer stem cells (also known as tumor perpetuating cells) and may be effectively leveraged to eliminate or silence the same. The ability to selectively reduce or eliminate cancer stem cells through the use of CLDN CARs as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed CLDN CARs and associated methods effectively overcome this inherent resistance to specifically eliminate, deplete, silence or promote the differentiation of such cancer stem cells thereby negating their ability to sustain or, significantly, re-induce underlying tumor growth.

Thus, it is particularly remarkable that CLDN CARs such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of exemplary signaling or co-stimulatory domains or regions or in the context of cancer stem cells or tumors comprising selected features and their interactions with the disclosed CLDN CARs, those skilled in the art will appreciate that the scope of the instant invention is not limited by such exemplary embodiments. Rather, the most expansive embodiments of the present invention and the appended claims are broadly and expressly directed to any chimeric antigen receptor comprising a binding domain that immunospecifically associates or binds to at least one claudin family member and their use in the treatment and/or prevention of a variety of CLDN associated or mediated disorders, including neoplastic or cell proliferative disorders, regardless of any particular mechanism of action, CAR construct or specifically targeted tumor, cellular or molecular component. In particularly preferred embodiments the CARs of the instant invention will immunospecifically bind to, or associate with, CLDN6.

To that end, and as demonstrated in the instant application, it has unexpectedly been found that the disclosed CLDN CARs can effectively be used to target and eliminate or otherwise incapacitate proliferative or tumorigenic cells and treat CLDN associated disorders (e.g., neoplasia). As used herein a “CLDN associated disorder” shall be held to mean any disorder or disease (including proliferative disorders) that is marked, diagnosed, detected or identified by a phenotypic aberration of CLDN genetic components or expression (“CLDN determinant”) during the course or etiology of the disease or disorder. In this regard a CLDN phenotypic aberration or determinant may, for example, comprise elevated or depressed levels of CLDN protein expression (e.g., CLDN6), abnormal CLDN protein expression on certain definable cell populations or abnormal CLDN protein expression at an inappropriate phase or stage of a cell lifecycle. Of course, it will be appreciated that similar expression patterns of genotypic determinants (e.g., mRNA transcription levels) of CLDN may also be used to classify, detect or treat CLDN disorders.

### I. Claudin (CLDN) Physiology

It has been found that CLDN phenotypic determinants are clinically associated with various proliferative disorders, including neoplasia, and that CLDN family proteins (including CLDN6) and variants or isoforms thereof provide useful tumor markers which may be exploited in the treatment of related diseases. In this regard the present invention provides a number of CLDN CAR constructs comprising an engineered anti-CLDN binding or targeting agent operably associated with one or more signaling domain(s) capable of inducing an immune response in a lymphocyte. As discussed in more detail below and set forth in the appended Examples, the disclosed anti-CLDN CARs are particularly effective at eliminating tumorigenic cells and therefore useful for the treatment and prophylaxis of certain proliferative disorders or the progression or recurrence thereof.

Moreover, it has been found that CLDN markers or determinants such as cell surface CLDN protein (e.g., CLDN6) are therapeutically associated with cancer stem cells (also known as tumor perpetuating cells) and may be effectively exploited to eliminate or silence the same. The ability to selectively reduce or eliminate cancer stem cells through the use of CLDN CARs as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed CARs and methods effectively overcome this inherent resistance and to specifically eliminate, deplete, silence or promote the differentiation of such cancer stem cells thereby negating their ability to sustain or re-induce the underlying tumor growth.

Claudins are integral membrane proteins comprising a major structural protein of tight junctions, the most apical cell-cell adhesion junction in polarized cell types such as those found in epithelial or endothelial cell sheets. Tight junctions are composed of strands of networked proteins that form continuous seals around cells to provide a physical but modulatable barrier to the transport of solutes and water in the paracellular space. The claudin family of proteins in humans is comprised of at least 23 members, ranging in size from 22-34 kDa. All claudins possess a tetraspanin topology in which both protein termini are located on the intracellular face of the membrane, resulting in the formation of two extracellular (EC) loops, EC1 and EC2. The EC loops mediate head-to-head homophilic, and for certain combinations of claudins, heterophilic interactions that lead to formation of tight junctions. The specific claudin-claudin interactions and claudin EC sequences are a key determinant of ion selectivity and tight junction strength (for example, see Nakano et al., 2009, PMID: 19696885). Typically, EC1 is about 50-60 amino acids in size, contains a conserved disulfide bond within a larger W—X(17-22)-W—X(2)-C—X(8-10)-C motif, and numerous charged residues that participate in ion channel formation (Turksen and Troy, 2004, PMID: 15159449). EC2 is smaller than EC1, being approximately 25 amino acids. Due to its helix-turn-helix conformation, it has been suggested that EC2 contributes to dimer or multimer formation of claudins on opposing cell membranes, although mutations in both loops may perturb complex formation. Claudin-claudin complexes in vitro may range in size from dimers to hexamers, depending upon the specific claudins involved (Krause et al., 2008, PMID: 18036336). Individual claudins show a range of tissue specific expression patterns, as well as developmentally regulated expression as determined by PCR analyses (Krause et al., 2008, PMID:18036336; Turksen, 2011, PMID:21526417).

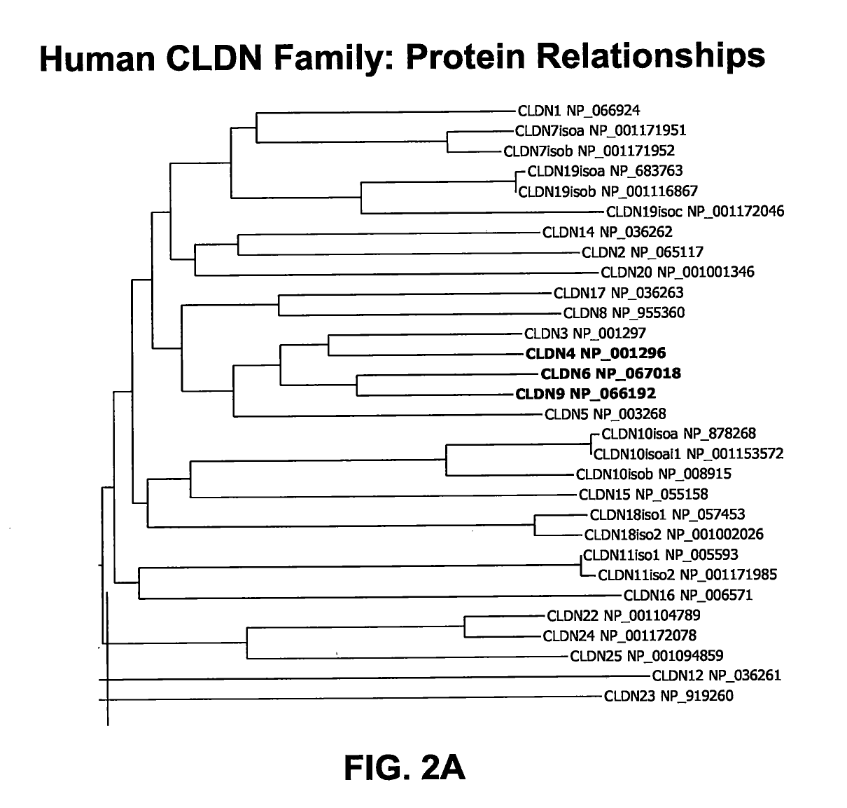

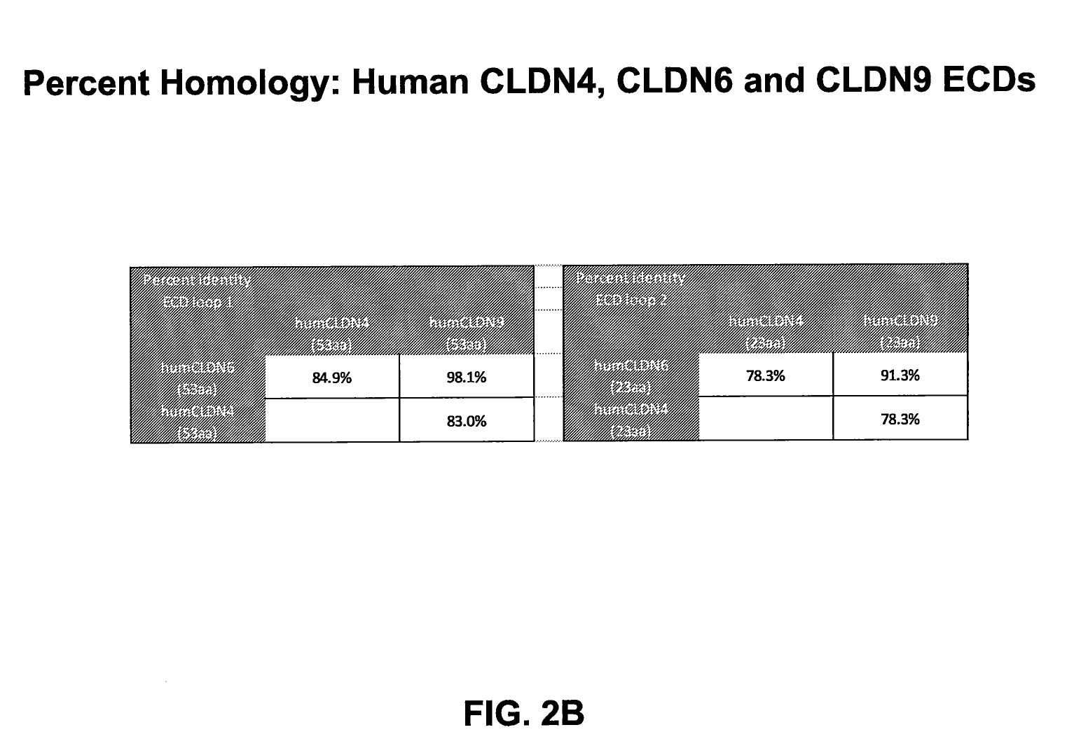

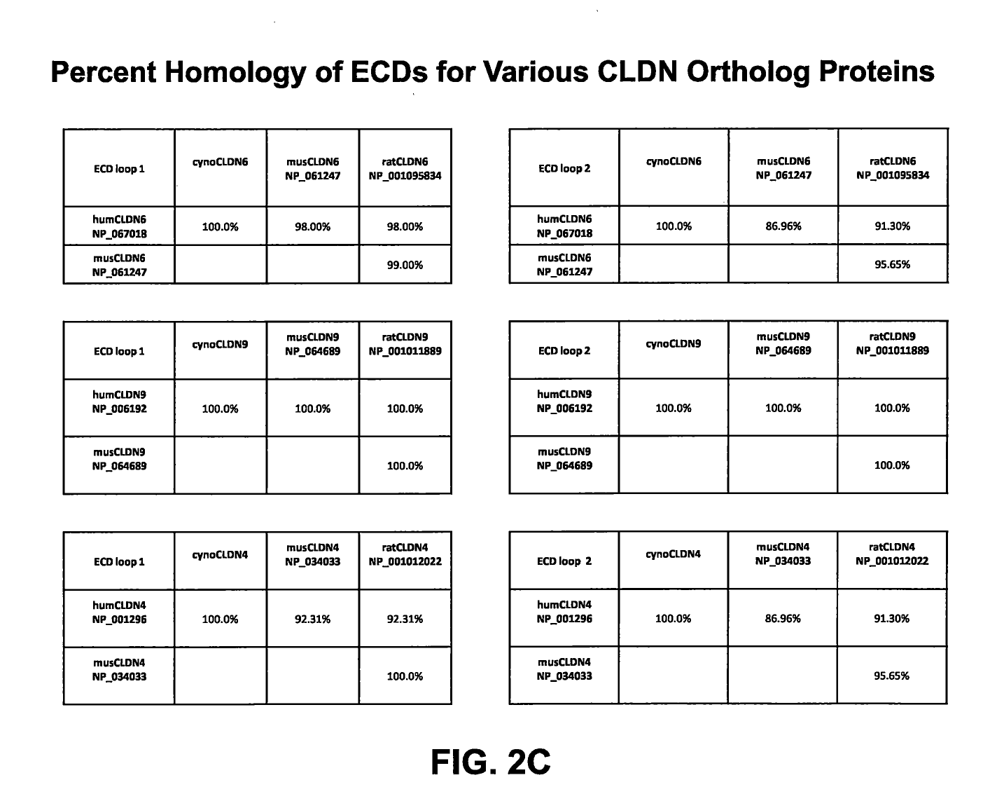

Sequence analysis can be used to construct phylogenetic trees for the claudin family members, indicating the relationship and degrees of relatedness of the protein sequences (FIG. 2A). For instance, it can be seen that the CLDN6 and CLDN9 proteins are closely related which, given the adjacent head-to-head location of their genes at the chromosomal location 16p3.3, is suggestive of an ancestral gene duplication. These similarities may translate to an ability of these family members to interact heterotypically. Similarly, the CLDN3 and CLDN4 proteins are closely related by sequence analysis, and their genes can be found in tandem at the chromosomal location 7r11.23. High homology in the EC1 or EC2 loops between certain family members (e.g. FIG. 2B) provides opportunity to develop antibodies that are multi-reactive with various claudin family members, while substantial homology between the ECD loop 1 and the ECD loop 2 of various species (FIG. 2C) allows for the development of cross-reactive antibodies that bind to selected orthologs.

CLDN6, also known as skullin, is a developmentally regulated claudin. Representative CLDN6 protein orthologs include, but are not limited to, human (NP_067018), chimpanzee (XP_523276), rhesus monkey (NP_001180762), mouse (NP_061247), and rat (NP_001095834). In humans, the CLDN6 gene consists of 2 exons spanning approximately 3.5 kBp at the chromosomal location 16p13.3. Transcription of the CLDN6 locus yields a mature 1.4 kB mRNA transcript (NM_021195), encoding a 219 amino acid protein (NP_061247). CLDN6 is expressed in ES cell derivatives committed to an epithelial fate (Turksen and Troy, 2001, PMID: 11668606), in the periderm (Morita et al., 2002, PMID: 12060405), and in the suprabasal level of the epidermis (Turkson and Troy, 2002, PMID: 11923212). It is also expressed in developing mouse kidney (Abuazza et al., 2006, PMID: 16774906), although expression is not detected in adult kidney (Reyes et al., 2002, PMID: 12110008). CLDN6 is also a coreceptor for hepatitis C virus, along with CLDN1 and CLDN9 (Zheng et al., 2007, PMID: 17804490).

CLDN9 is the most closely related family member to CLDN6. Representative CLDN9 protein orthologs include, but are not limited to, human (NP_066192), chimpanzee (XP_003314989), rhesus monkey (NP_001180758), mouse (NP_064689), and rat (NP_001011889). In humans, the CLDN9 gene consists of a single exon spanning approximately 2.1 kBp at the chromosomal locus 16p13.3. Transcription of the intronless CLDN9 locus yields a 2.1 kB mRNA transcript (NM_020982), encoding a 217 amino acid protein (NP_0066192). CLDN9 is expressed in various structures of the inner ear (Kitarjiri et al., 2004, PMID:14698084; Nankano et al., 2009, PMID: 19696885), the cornea (Ban et al., 2003, PMID:12742348), the liver (Zheng et al., 2007, PMID:17804490) and developing kidney (Abuazza et al., 2006, PMID:16774906). Consistent with its expression in the cochlea, animals expressing a CLDN9 protein with a missense mutation show defects in hearing likely due to altered paracellular K⁺permeability with consequent perturbation of ion currents critical for depolarization of hair cells involved in sound detection. Expression of CLDN9 in cells of the inner ear is specifically localized to a subdomain underneath more apical tight-junction strands formed by other claudins, indicating that not all claudins in normal tissues are found in the most apical and accessible tight junctions (Nankano et al., 2009, PMID: 19696885). In contrast to the results in the cochlea, mice expressing missense CLDN9 showed no signs of hepatic or renal defects (Nankano et al., 2009, PMID: 19696885).

CLDN4 is also known as the Clostridium perfringens enterotoxin receptor, due to its high affinity binding of this toxin responsible for food poisoning and other gastrointestinal illnesses. Representative CLDN4 protein orthologs include, but are not limited to, human (NP_001296), chimpanzee (XP_519142), rhesus monkey (NP_001181493), mouse (NP_034033), and rat (NP_001012022). In humans, the intronless CLDN4 gene spans approximately 1.82 kBp at the chromosomal location 17q11.23. Transcription of the CLDN4 locus yields a 1.82 kB mRNA transcript (NM_001305), encoding a 209 amino acid protein (NP_001296). Consistent with the ability of CLDN4 to bind a toxin produced by a gastrointestinal pathogen, CDLN4 expression can be detected throughout the GI tract as well as in prostate, bladder, breast, and lung (Rahner et al., 2001, PMID:11159882; Tamagawa et al., 2003, PMID:12861044; Wang et al., 2003, PMID:12600828; Nichols et al., 2004, PMID:14983936).

Although claudins are important in the function and homeostasis of normal tissues, tumor cells frequently exhibit abnormal tight junction function. This may be linked to disregulated expression and/or localization of claudins as a consequence of the dedifferentiation of tumor cells, or the requirement of rapidly growing cancerous tissues to efficiently absorb nutrients within a tumor mass with abnormal vascularization (Morin, 2005, PMID: 16266975). Individual claudin family members may be up-regulated in certain cancer types, yet down-regulated in others. For example, CLDN3 and CLDN4 expression is elevated in certain pancreatic, breast and ovarian cancers, yet may be lower in other breast (e.g., “claudin-low”) carcinomas. Claudin proteins may be particularly good targets for CARs since it is known that claudins undergo endocytosis, turnover time of some claudins is short relative to other membrane proteins (Van Itallie et al., 2004, PMID: 15366421), claudin expression is disregulated in cancer cells and tight junctions structures among tumor cells are disrupted in cancer cells. These properties may afford more opportunities for activated lymphocytes to bind claudin proteins in neoplastic but not in normal tissues. Although binding domains specific to individual claudins may be useful, it is also possible that polyreactive claudin CARs would be more likely to facilitate the delivery of payloads to a broader patient population. Specifically, polyreactive claudin CARs may permit more efficient targeting of cells expressing multiple claudin proteins due to higher aggregate antigen density, reduce the likelihood of escape of tumor cells with low levels of antigen expression of any individual claudin, and as can be seen in the expression examples below, expand the number of therapeutic indications for a single CLDN CAR.

### II. Cancer Stem Cells

As alluded to above it has surprisingly been discovered that aberrant CLDN expression (genotypic and/or phenotypic) is associated with various tumorigenic cell subpopulations. In this respect the present invention provides CLDN CAR mediated therapeutic regimens that may be particularly useful for targeting such cells (e.g., cancer stem cells), thereby facilitating the treatment, management or prevention of neoplastic disorders. Thus, in preferred embodiments the disclosed CLDN CAR may be advantageously be used to reduce tumor initiating cell frequency in accordance with the present teachings and thereby facilitate the treatment or management of proliferative disorders.

According to the current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which make up 0.1-40% (more typically 0.1-10%) of a tumor's cell population, have the ability to form tumors. Tumorigenic cells encompass both tumor perpetuating cells (TPCs), referred to interchangeably as cancer stem cells (CSCs) and tumor progenitor cells (TProgs).

CSCs, like normal stem cells that support cellular hierarchies in normal tissue, are able to self-replicate indefinitely while maintaining the capacity for multilineage differentiation. CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice.

Tprogs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because Tprogs are typically only capable of a finite number of cell divisions as demonstrated by serial transplantation of low numbers of highly purified Tprog into immunocompromised mice. Tprogs may further be divided into early Tprogs and late Tprogs, which may be distinguished by phenotype (e.g., cell surface markers) and their different capacities to recapitulate tumor cell architecture. While neither can recapitulate a tumor to the same extent as CSCs, early Tprogs have a greater capacity to recapitulate the parental tumor's characteristics than late Tprogs. Notwithstanding the foregoing distinctions, it has been shown that some Tprog populations can, on rare occasion, gain self-renewal capabilities normally attributed to CSCs and can themselves become CSCs.

CSCs exhibit higher tumorigenicity and are relatively more quiescent than: (i) Tprogs (both early and late Tprogs); and (ii) non-tumorigenic cells such as tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are more resistant to conventional therapies and regimens than the faster proliferating Tprogs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. These properties in CSCs constitute a key reason for the failure of standard oncology treatment regimens to ensure long-term benefit for most patients with advanced stage neoplasia because standard chemotherapy does not target the CSCs that actually fuel continued tumor growth and recurrence.

It has surprisingly been discovered that CLDN expression (including CLDN6 expression) is associated with various tumorigenic cell populations. The invention provides CLDN CARs that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, or otherwise inhibit (collectively, “inhibit”) tumorigenic cells, thereby facilitating the treatment, management and/or prevention of proliferative disorders (e.g. cancer). Advantageously, the novel CLDN CARs of the invention may be selected so they preferably reduce the frequency or tumorigenicity of tumorigenic cells upon administration to a subject regardless of the form of the CLDN determinant (e.g., isotype a or b). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition of the tumorigenic cells, modification of their potential (for example, by induced differentiation or niche disruption) or otherwise interfering with the ability of tumorigenic cells to influence the tumor environment or other cells, allows for the more effective treatment of CLDN associated disorders by inhibiting tumorigenesis, tumor maintenance and/or metastasis and recurrence.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

Listed below are markers that have been associated with CSC populations and, in some cases, have been used to isolate or characterize CSCs: ABCA1, ABCA3, ABCG2, ADCY9, ADORA2A, AFP, AXIN1, B7H3, BCL9, Bmi-1, BMP-4, C20orf52, C4.4A, carboxypeptidase M, CAV1, CAV2, CD105, CD133, CD14, CD16, CD166, CD16a, CD16b, CD2, CD20, CD24, CD29, CD3, CD31, CD324, CD325, CD34, CD38, CD44, CD45, CD46, CD49b, CD49f, CD56, CD64, CD74, CD9, CD90, CEACAM6, CELSR1, CPD, CRIM1, CX3CL1, CXCR4, DAF, decorin, easyh1, easyh2, EDG3, eed, EGFR, ENPP1, EPCAM, EPHA1, EPHA2, FLJ10052, FLVCR, FZD1, FZD10, FZD2, FZD3, FZD4, FZD6, FZD7, FZD8, FZD9, GD2, GJA1, GLI1, GL12, GPNMB, GPR54, GPRC5B, IL1R1, IL1RAP, JAM3, Lgr5, Lgr6, LRP3, LY6E, MCP, mf2, mllt3, MPZL1, MUC1, MUC16, MYC, N33, Nanog, NB84, nestin, NID2, NMA, NPC1, oncostatin M, OCT4, OPN3, PCDH7, PCDHA10, PCDHB2, PPAP2C, PTPN3, PTS, RARRES1, SEMA4B, SLC19A2, SLC1A1, SLC39A1, SLC4A11, SLC6A14, SLC7A8, smarcA3, smarcD3, smarcE1, smarcA5, Sox1, STAT3, STEAP, TCF4, TEM8, TGFBR3, TMEPAI, TMPRSS4, transferrin receptor, TrkA, WNT10B, WNT16, WNT2, WNT2B, WNT3, WNT5A, YY1 and β-catenin. See, for example, Schulenburg et al., 2010, PMID: 20185329, U.S. Pat. No. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD46^{hi}CD324⁺CD66c⁺, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺ as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes.

“Positive,” “low” and “negative” expression levels as they apply to markers or marker phenotypes are defined as follows. Cells with negative expression (i.e.“−”) are herein defined as those cells expressing less than, or equal to, the 95^{th} percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95^{th} percentile of expression observed with an isotype control antibody using the FMO staining procedure described above are herein defined as “positive” (i.e.“+”). As defined herein there are various populations of cells broadly defined as “positive.” A cell is defined as positive if the mean observed expression of the antigen is above the 95^{th} percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99^{th} percentile may preferably be used as a demarcation point between negative and positive FMO staining and in particularly preferred embodiments the percentile may be greater than 99%.

The CD46^{hi}CD324⁺ marker phenotype and those exemplified immediately above may be used in conjunction with standard flow cytometric analysis and cell sorting techniques to characterize, isolate, purify or enrich TIC and/or TPC cells or cell populations for further analysis.

The ability of the antibodies of the current invention to reduce the frequency of tumorigenic cells can therefore be determined using the techniques and markers described above. In some instances CLDN CAR may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed adoptive immunotherapy may reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. Chimeric Antigen Receptor Therapy

Cancer immunotherapies aim to harness the power of the human immune system to eradicate tumors via the activity of cytotoxic lymphocytes (comprising both T-lymphocytes and NK cells). That cytotoxic lymphocyte-mediated immune responses could lead to the eradication of residual tumor cells was inferred from studies that compared relapse rates in leukemia patients that had undergone various types of transplantation: a significant reduction in relapse rates was observed for patients receiving non-T-cell depleted marrow in allogeneic transplants from HLA identical siblings versus those receiving syngenic transplants, and this effect could be attributed to other T-cell mediated actions beyond graft-versus-host disease responses. However, clinically effective adoptive transfer of anti-tumor T-cells has been hampered by the fact that most tumor antigens are self-antigens, and therefore are poorly immunogenic. Negative selection of T-cells bearing high-affinity T-cell receptors (TCRs) recognizing self-antigens takes place in the thymus during development, resulting in central tolerance and selection for T-cells having low-avidity recognition of the tumor/self-antigen. These lower avidity T-cells then have consequent weak activation of anti-tumor T-cell function and limited persistence. Genetically engineered cytotoxic lymphocytes are being deployed in two major approaches to circumvent this tolerance/low avidity block to strong anti-tumor T-cell activation. In the first approach, affinity-enhanced TCR recognizing tumor antigens are artificially introduced into T-cells using molecular genetic engineering techniques. This approach is limited by several factors, including difficulty in expressing the affinity-enhanced TCR at levels approaching wild-type TCR expression, the potential for mispairing of TCR chains which arises when introducing additional sets of TCR genes into a native T-cell, and the ability of tumor cells to evade MHC-restricted TCR recognition by down-regulating MHC molecules.

A second approach to the genetic engineering of cytotoxic lymphocytes is introduction of an artificial, non-MHC-restricted chimeric antigen receptor (CAR) into various lymphocyte populations. This is most typically achieved by harvesting bulk lymphocyte populations which are cultured, stimulated and expanded ex vivo prior to transduction with retroviral or lentiviral vectors encoding the CAR molecule. Like a native TCR, the CAR must possess the ability to specifically and selectively recognize a target antigen, and then upon binding to this antigen, transduce the appropriate signals to the lymphocyte to stimulate effector functions and/or the cytokine production necessary for a sustained anti-tumor immunological response. The concept of CAR-modified T-cells arose from studies in which it was observed that the cytoplasmic ITAM domain of the CD3ζ chain could activate T-cells when expressed independently from the TCR:CD3 protein complex, particularly when the CD3ζ ITAM domain was fused to a heterologous extracellular and transmembrane domain. A first-generation CD4-CD3ζ CAR was transduced into T-cells and tested in HIV patients. Follow up studies showed these engineered CAR-T cells persisted for up to a decade after infusion, indicative of some proliferation and persistence of the engineered cells. Subsequently, anti-tumor CARs were constructed by combining in a single recombinant molecule a scFv domain and a transmembrane domain with the cytoplasmic domain of the CD3ζ chain, and it could be shown the antigen recognition of these engineered CAR-T cells was redirected to reflect the specificity of the scFv (U.S. Pat. No. 7,446,179). These first generation scFv-directed CAR-T cells were capable of acting as non-MHC restricted cytotoxic lymphocytes, recognizing native tumor antigen rather than processed peptides, and promoting lysis of tumor cells expressing the native antigen.

While many of the first generation scFv-directed CAR-T cells showed expected effects in vitro, in vivo studies in cancer patients were disappointing for their lack of anti-tumor effects and lack of CAR-T persistence. As T-cell biology has become better understood, it has become clear that T-cell populations are comprised of short lived effector cells, longer-lived central and peripheral memory T-cells, as well as regulatory T-cells (Tregs) that interact with the other T-cell subpopulations. Central to function of these populations are the role of costimulatory signals in inducing persistent activation of resting naïve or memory T-cells via cytokine production, as well as the role co-stimulation provides in preventing anergy, a state of T-cell non-responsiveness that may potentially arise from exclusive TCR:CD3ζ signaling in the absence of co-stimulatory signals. In particular, various co-stimulatory signals from proteins such as CD28, OX40, CD27, CD137/4-1BB, CD2, CD3, CD11a/CD18, CD54 and CD58 may be beneficial for optimal levels of cytokine production, proliferation and clonal expansion, and induction of cytolytic activity. Of these, CD28 is perhaps the best understood co-stimulatory signal, and CD28 co-stimulation has been shown to augment cytokine release by antigen activated CAR-T cells. Similarly, co-stimulatory signaling through CD137/4-1BB has been shown to enhance native T-cell proliferation, and may contribute to longer persistence of CAR-T in vivo. Therefore, so called second generation CAR constructs have been designed in which various additional signaling domains from these molecules have been added in tandem to the CD3ζ domain (U.S. Pat. Nos. 5,686,281 and 8,399,645). So called third generation CAR molecules including three or more signaling domains (e.g., CD3ζ and two co-stimulatory signaling domains) are also reportedly under development.

Several second generation CAR-T cells directed to the CD19 antigen have been shown to have strong antitumor effects, as well as substantial persistence, in patients with hematological malignancies. A major frontier for CAR-T therapies is applications in the treatment of solid tumors. With respect to the instant invention it has surprisingly been discovered that anti-CLDN binding domains may be advantageously integrated with each of the aforementioned chimeric antigen receptors and adoptive immunotherapies to provide effective antineoplastic treatments that overcome some of the previous limitations.

### IV. Chimeric Antigen Receptors

As alluded to above the CARs of the instant invention generally comprise an extracellular domain comprising a CLDN binding domain, a transmembrane domain and an intracellular signaling domain that activates certain lymphocytes and generates an immune response directed to CLDN positive tumor cells. More generally, the disclosed chimeric antigen receptors comprise an ectodomain and an endodomain each as defined by the host cell wall. In this regard the terms “ectodomain” or “extracellular domain” will refer to the portion of the CAR polypeptide outside of the cell or exterior to the membranous lipid bilayer, which may comprise the antigen recognition (e.g., CLDN) binding domains, an optional hinge region, and any spacer domains exterior the to the amino acid residues physically spanning the membrane. Conversely the terms “endodomain” or “intracellular domain” will refer to the portion of the CAR polypeptide inside the cell or interior to the membranous lipid bilayer, which may comprise any spacer domains interior to the amino acid residues physically spanning the membrane, as well as the intracellular signaling domain.

A. CLDN Binding Domains

1. Binding Domain Structure

As discussed extensively throughout the instant disclosure, chimeric antigen receptors comprising an anti-CLDN binding domain may advantageously be used to provide targeted therapies for various proliferative disorders. It will be appreciated that compatible anti-CLDN binding domains may comprise anti-CLDN antibodies or immunoreactive fragments thereof. In certain embodiments intact antibodies or antibodies comprising at least some portion of the fc or constant domain comprise the CLDN binding domain (see, for example, U.S.P.N. 2015/0139943). In particularly preferred embodiments, and as demonstrated in the examples appended hereto, the anti-CLDN binding domain may comprise a scFv derived from a monoclonal antibody (including humanized or CDR grafted monoclonal antibodies) that binds to CLDN. Compatible antibodies that may be used to provide CLDN binding domains consistent with the instant invention are discussed in more detail immediately below. For the purposes of the instant application the terms “binding domain” and “antibody” or “antibody fragment” may be used interchangeably unless otherwise contextually dictated.

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

An “intact antibody” typically comprises a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As alluded to above the term “antibody” should be construed generally and includes polyclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. scFv and ScFvFc); and derivatives thereof including Fc fusions and other modifications, and any other immunoreactive immunoglobulin molecule so long as it exhibits preferential association or binding with a CLDN determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2) and all immunoreactive fragments thereof. Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains. In short, any such antibody that binds to or associates with human CLDN is compatible with the teachings herein and may be used as the binding domain component for the disclosed chimeric antigen receptors.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. Of particular interest scFv constructs (for single chain fragment variable), which can be obtained by genetic engineering as discussed more extensively below, join VH and the VL regions (preferably from the same antibody), though a peptide linker. Depending on the desired conformation it will be appreciated that the peptide linker may be of various lengths.

As used herein, the assignment of amino acids to each domain, framework region and CDR may be in accordance with one of the numbering schemes provided by Kabat et aL (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. The amino acid residues which comprise CDRs as defined by Kabat, Chothia, MacCallum (also known as Contact) and AbM schemes, as obtained from the Abysis website database (infra.), are set out below.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A.C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably antibody sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of myeloma protein Eu, which reportedly was the first human IgG1 sequenced. The EU index of Edelman is also set forth in Kabat et aL, 1991 (supra.). Thus, the terms “EU index as set forth in Kabat” or “EU index of Kabat” or “EU index” in the context of the heavy chain refers to the residue numbering system based on the human IgG1 Eu antibody of Edelman et al. as set forth in Kabat et al., 1991 (supra.) The numbering system used for the light chain constant region amino acid sequence is similarly set forth in Kabat et al., (supra.) An exemplary kappa light chain constant region amino acid sequence compatible with the present invention is set forth immediately below:

(SEQ ID NO: 1)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Similarly, an exemplary IgG1 heavy chain constant region amino acid sequence compatible with the present invention is set forth immediately below:

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

The disclosed constant region sequences, or variations or derivatives thereof, may be operably associated with the disclosed heavy and light chain variable regions using standard molecular biology techniques to provide antibodies (full length or immunoreactive fragments comprising partial fc regions) that may be used as such or incorporated in CLDN CARs of the invention (preferably as part of the transmembrane domain).

More generally, the anti-CLDN binding domain component of compatible CARs may be generated from any antibody that specifically recognizes or associates with at least one CLDN determinant (e.g., CLDN4, CLDN6, CLDN9) or some combination thereof. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In certain preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a CLDN family member protein, or any of its splice variants, isoforms, homologs or family members, or specific domains, regions or epitopes thereof. An “antigen”, “immunogenic determinant”, “antigenic determinant” or “immunogen” means any protein or any fragment, region or domain thereof that can stimulate an immune response when introduced into an immunocompetent animal and is recognized by the antibodies produced from the immune response. The presence or absence of the CLDN determinants contemplated herein may be used to identify a cell, cell subpopulation or tissue (e.g., tumors, tumorigenic cells or CSCs).

2. Antibody Generation and Production

Antibodies compatible with the invention can be produced using a variety of methods known in the art and any such antibodies may be further modified to provide the binding domain of the anti-CLDN chimeric antigen receptors of the instant invention.

a. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

Any form of antigen, or cells or preparations containing the antigen, can be used to generate an antibody that is specific for a determinant. The term “antigen” is used in a broad sense and may comprise any immunogenic fragment or determinant of the selected target including a single epitope, multiple epitopes, single or multiple domains or the entire extracellular domain (ECD). The antigen may be an isolated full-length protein, a cell surface protein (e.g., immunizing with cells expressing at least a portion of the antigen on their surface), or a soluble protein (e.g., immunizing with only the ECD portion of the protein). The antigen may be produced in a genetically modified cell. Any of the aforementioned antigens may be used alone or in combination with one or more immunogenicity enhancing adjuvants known in the art. The DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

b. Monoclonal Antibodies

In selected embodiments, the invention contemplates use of monoclonal antibodies. The term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques including hybridoma techniques, recombinant techniques, phage display technologies, transgenic animals (e.g., a XenoMouse®) or some combination thereof. For example, in preferred embodiments monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+ Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following generation of a number of monoclonal antibodies that bind specifically to a determinant, particularly suitable antibodies may be selected through various screening processes, based on, for example, affinity for the determinant or rate of internalization. In particularly preferred embodiments monoclonal antibodies produced as described herein may be used as “source” antibodies and further modified to provide effective CLDN binding domains that may be associated with the disclosed CARs. For example the source antibody may be manipulated to provide scFvs or other fragments, improve affinity for the target, improve its production in cell culture, reduce immunogenicity in vivo, create multispecific constructs, etc. A more detailed description of monoclonal antibody production and screening is set out below and in the appended Examples.

c. Human Antibodies

Antibodies compatible with the instant invention may comprise fully human antibodies. The term “human antibody” refers to an antibody (preferably a monoclonal antibody) which possesses an amino acid sequence that corresponds to that of an antibody produced by a human and/or has been made using any of the techniques for making human antibodies described below.

In one embodiment, recombinant human antibodies may be isolated by screening a recombinant combinatorial antibody library prepared using phage display. In one embodiment, the library is a scFv phage or yeast display library, generated using human VL and VH cDNAs prepared from mRNA isolated from B-cells.

Human antibodies can also be made by introducing human immunoglobulin loci into transgenic animals, e.g., mice in which the endogenous immunoglobulin genes have been partially or completely inactivated and human immunoglobulin genes have been introduced. Upon challenge antibody generation is observed which closely resembles that seen in humans in all respects, including gene rearrangement, assembly and fully human antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, 1995, PMID: 7494109). Alternatively, a human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., 1991, PMID: 2051030; and U.S. Pat. No. 5,750,373. As with other monoclonal antibodies such human antibodies may be used as source antibodies.

d. Antibody Production and Engineering

Antibodies and fragments thereof may be produced or modified using genetic material obtained from antibody producing cells and recombinant technology (see, for example, Berger and Kimmel, Guide to Molecular Cloning Techniques, Methods in Enzymology vol. 152 Academic Press, Inc., San Diego, Calif.; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

As will be discussed in more detail below another aspect of the invention pertains to nucleic acid molecules that encode the CLDN binding domains and CARs of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCI banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In a preferred embodiment, the nucleic acid is a cDNA molecule.

Nucleic acids of the invention can be obtained and manipulated using standard molecular biology techniques. For antibodies expressed by hybridomas (e.g., hybridomas prepared as set forth in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or preferably to a nucleotide sequence encoding a CLDN specific scFv. In these manipulations, a VL- or VH-encoding DNA fragment is operatively linked to another DNA fragment encoding another protein, such as an antibody constant region or a flexible linker. The term “operatively linked” or “operably linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. In this respect an exemplary compatible kappa light chain constant region is set forth in SEQ ID NO: 1.

Contemplated herein are certain polypeptides (e.g. antibody variable regions) that exhibit “sequence identity”, sequence similarity” or “sequence homology” to the polypeptides of the invention. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci.,4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in preferred embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid cells or CSCs).

3. Derived Antibodies as CLDN Binding Domains

Once the source antibodies have been generated, selected and isolated as described above, they may be further altered to provide anti-CLDN CAR binding domain components compatible with the teachings herein. Preferably the source antibodies are modified or altered using known molecular engineering techniques to provide derived binding domain components having the desired therapeutic properties.

a. Chimeric and Humanized Antibodies

As discussed above selected embodiments of the invention comprise murine monoclonal antibodies that immunospecifically bind to CLDN and, for the purposes of the instant disclosure, may be considered “source” antibodies for CLDN binding domains. In selected embodiments, CLDN binding domains compatible with the invention can be derived from such source antibodies through optional modification of the constant region and/or the antigen binding amino acid sequences of the source antibody. In certain embodiments an antibody is derived from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor binding domain construct to provide the derivative CLDN binding domain (e.g. chimeric or humanized binding domains). These derived binding domains can be generated using standard molecular biological techniques as described below, such as, for example, to provide an scFv; to improve affinity for the determinant; to improve antibody stability; to improve expression; to reduce immunogenicity in vivo; to reduce toxicity or to facilitate transmission of a signal. Such antibodies may also be derived from source antibodies through modification of the mature molecule (e.g., glycosylation patterns or pegylation) by chemical means or post-translational modification.

In one embodiment, the chimeric binding regions of the invention are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some preferred embodiments chimeric antibodies of the instant invention may comprise all or most of the selected murine heavy and light chain variable regions operably linked to all or part of human light and heavy chain constant regions. In other particularly preferred embodiments, CLDN binding domains may be “derived” from the mouse antibodies disclosed herein.

In other embodiments, the chimeric binding domains of the invention are “CDR grafted” where the CDRs (as defined using Kabat, Chothia, McCallum, etc.) are derived from a particular species or belonging to a particular antibody class or subclass, while the remainder of the binding region is derived from an antibody from another species or belonging to another antibody class or subclass. For use in humans, one or more selected rodent CDRs (e.g., mouse CDRs) may be grafted into a human acceptor binding domain (i.e., with human framework regions), replacing one or more of the naturally occurring CDRs of the human antibody. These constructs generally have the advantages of providing effective binding while reducing unwanted immune responses to the binding domain by the subject. In particularly preferred embodiments the CDR grafted binding domains will comprise one or more CDRs obtained from a mouse incorporated in a human framework sequence.

Similar to the CDR-grafted binding domain is a “humanized” binding domain. As used herein, a “humanized” binding domain is a human binding domain (acceptor domain generally comprising human framework regions) comprising one or more amino acid sequences (e.g. CDR sequences) derived from one or more non-human antibodies (a donor or source antibody). In certain embodiments, “back mutations” can be introduced into the humanized binding domain, in which residues in one or more FRs of the variable region of the recipient human binding domain are replaced by corresponding residues from the non-human species donor antibody. Such back mutations may to help maintain the appropriate three-dimensional configuration of the grafted CDR(s) and thereby improve affinity and binding domain stability. Antibodies from various donor species may be used including, without limitation, mouse, rat, rabbit, or non-human primate. Furthermore, humanized antibodies or fragments may comprise new residues that are not found in the recipient antibody or in the donor antibody to, for example, further refine antibody performance. CDR grafted and humanized antibodies (and related CLDN binding domains) compatible with the instant invention and comprising the source murine antibodies set forth in the Examples below may therefor readily be provided without undue experimentation using the prior art techniques as set forth herein.

Various art-recognized techniques can further be used to determine which human sequences to use as acceptor antibodies to provide humanized constructs in accordance with the instant invention. Compilations of compatible human germline sequences and methods of determining their suitability as acceptor sequences are disclosed, for example, in Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638 each of which is incorporated herein in its entirety. The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody (or binding domain) may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

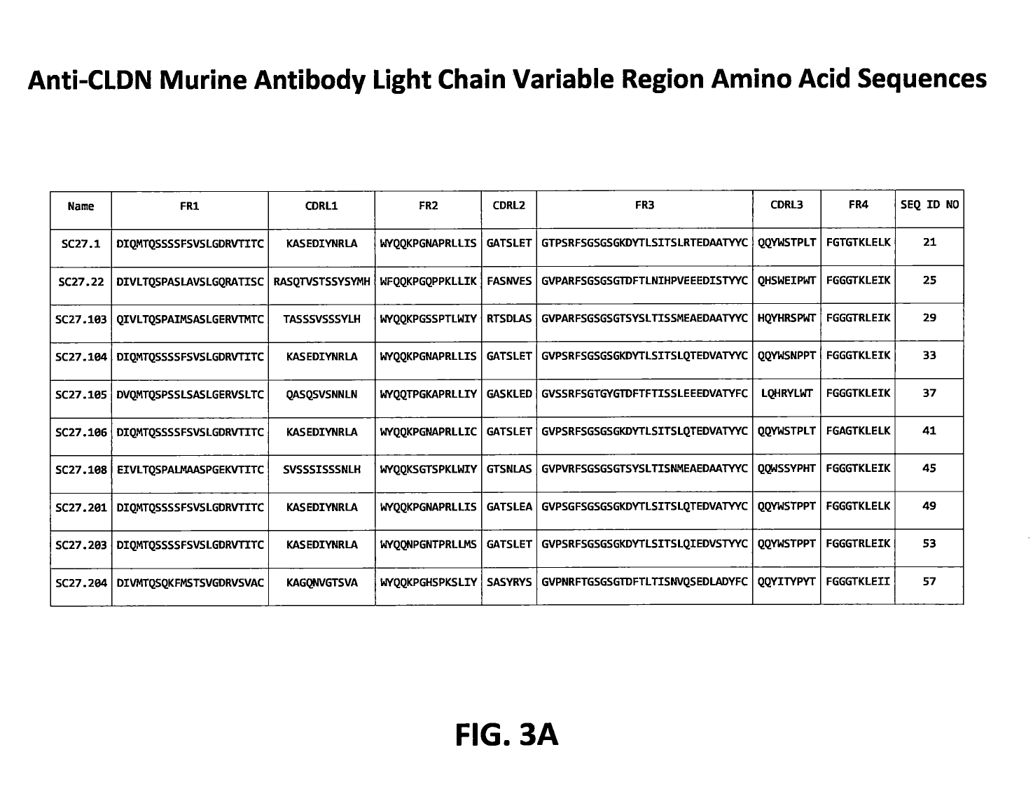

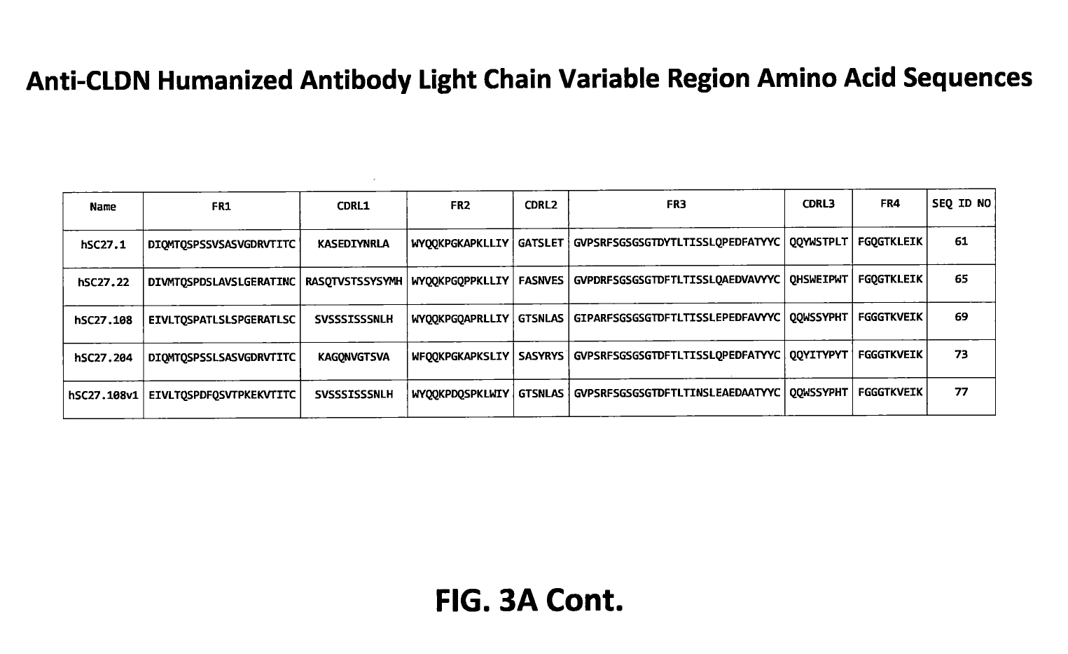

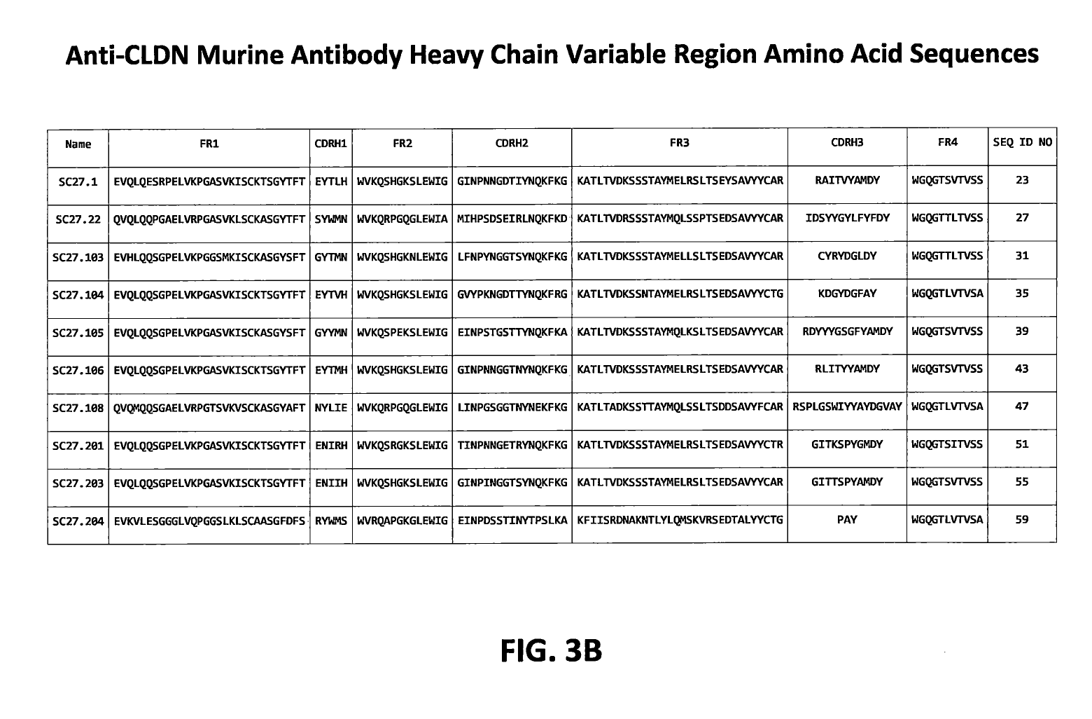

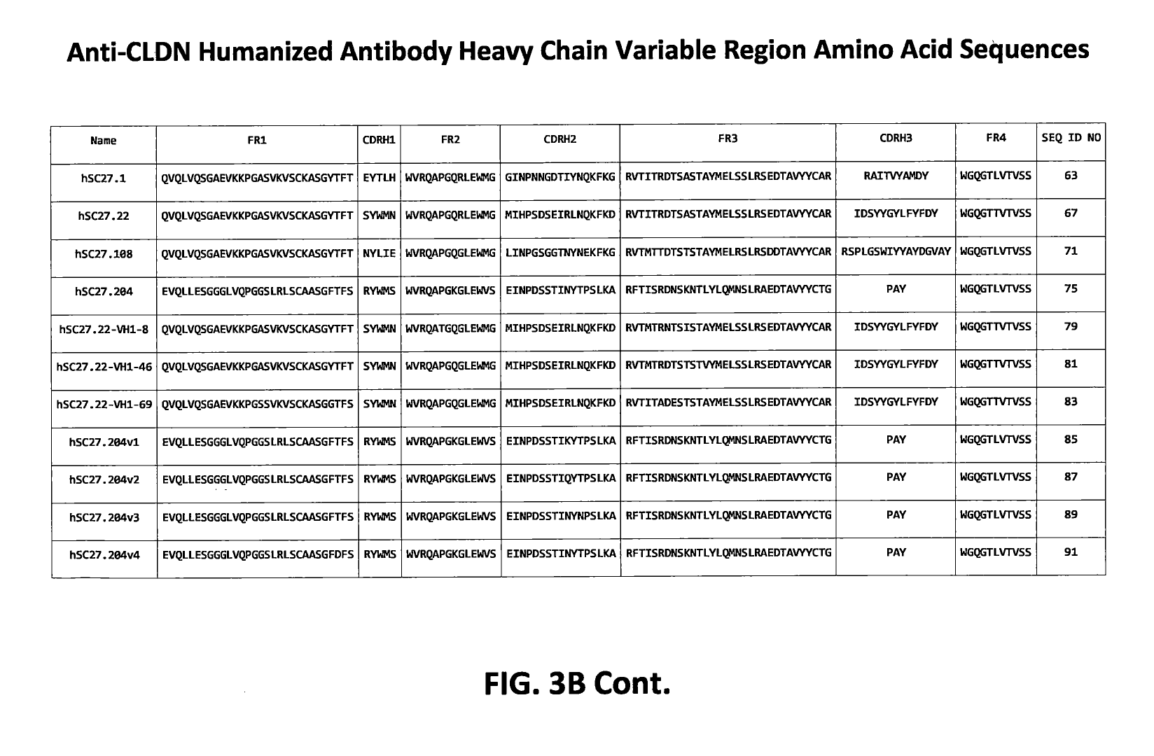

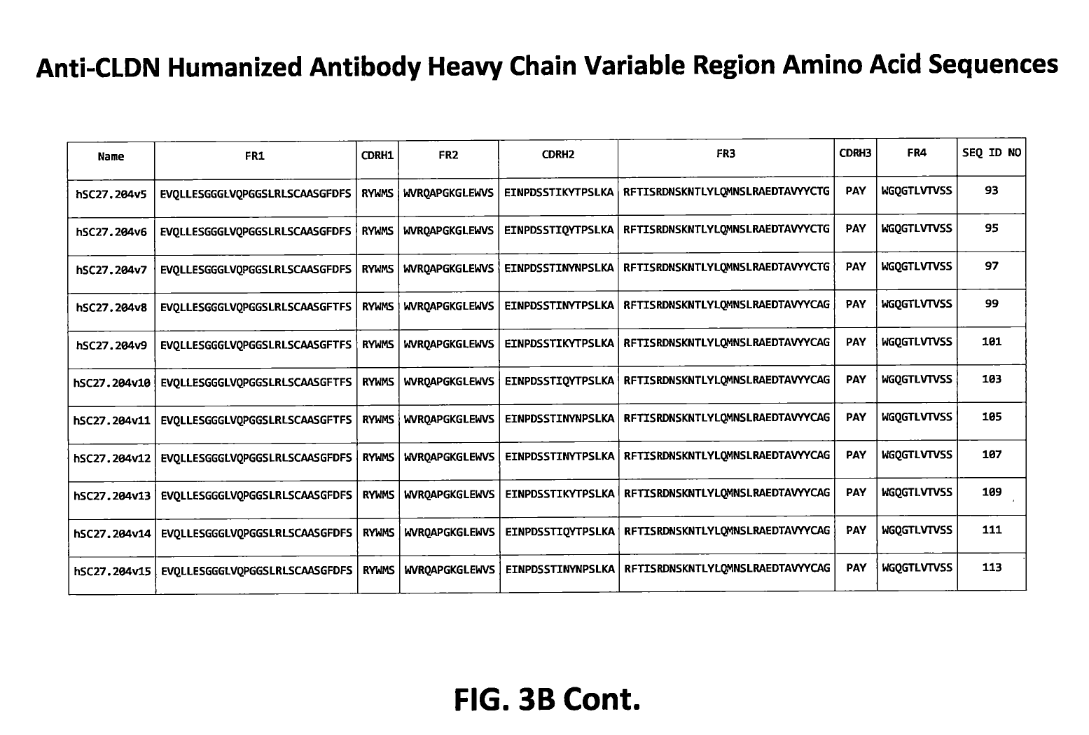

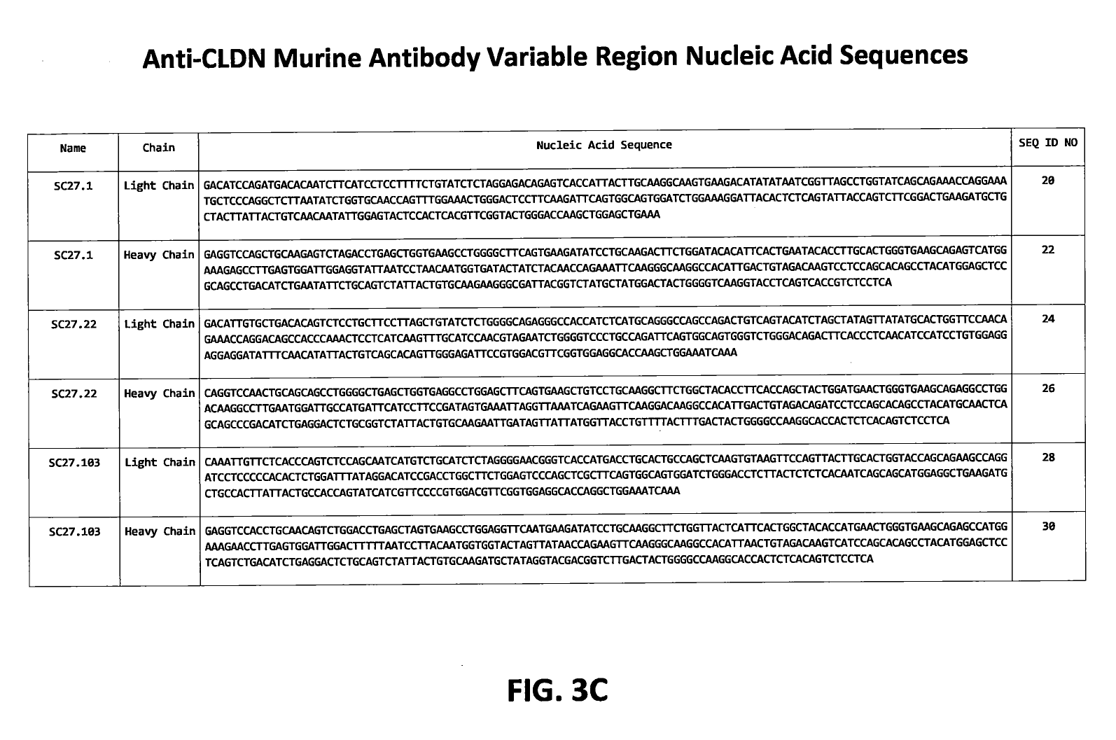

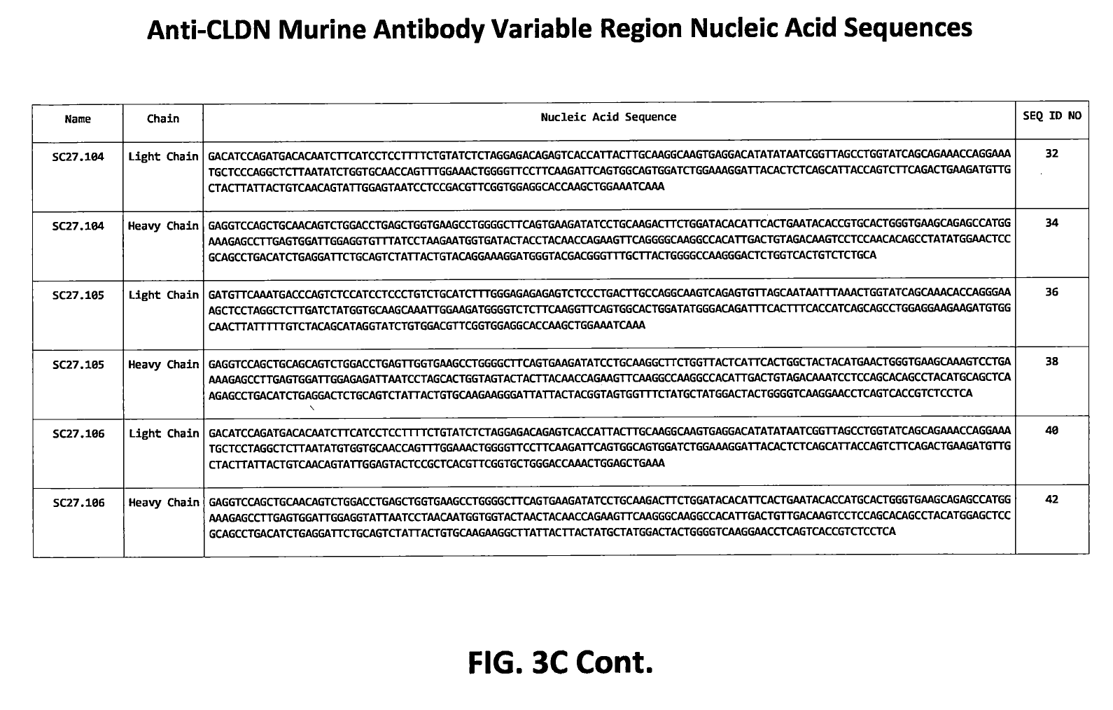

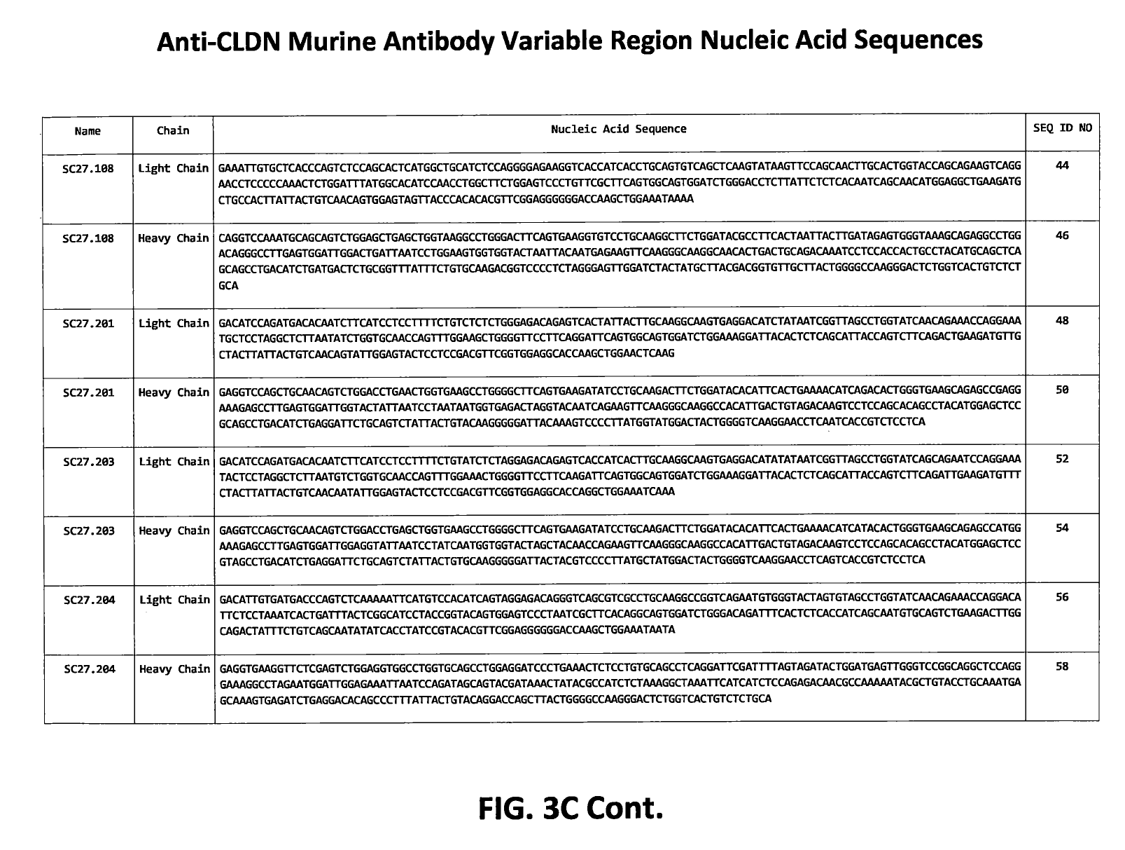

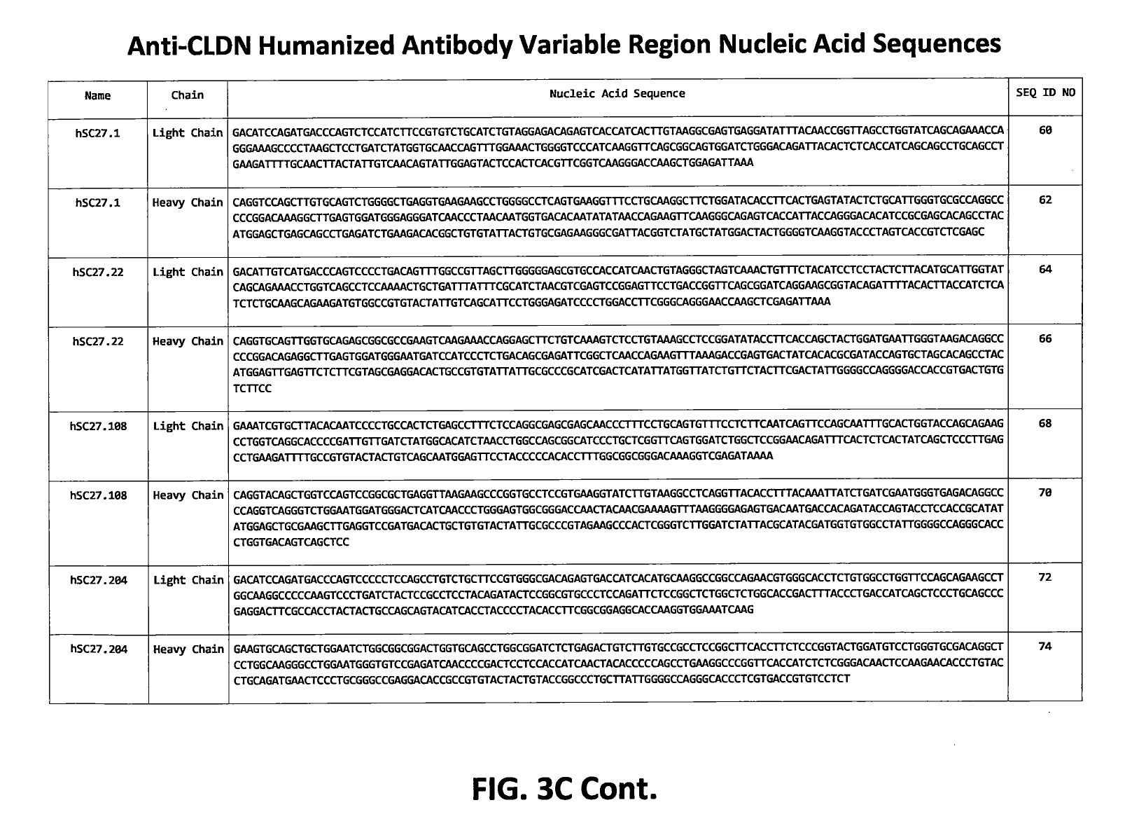

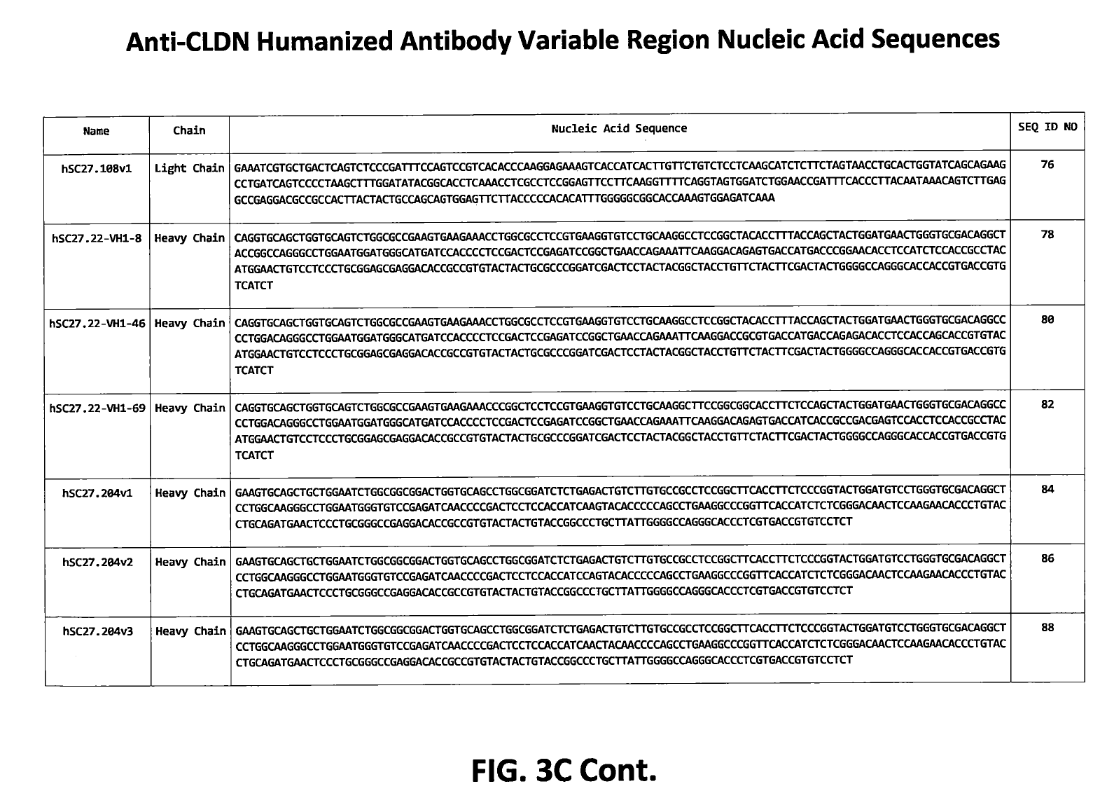

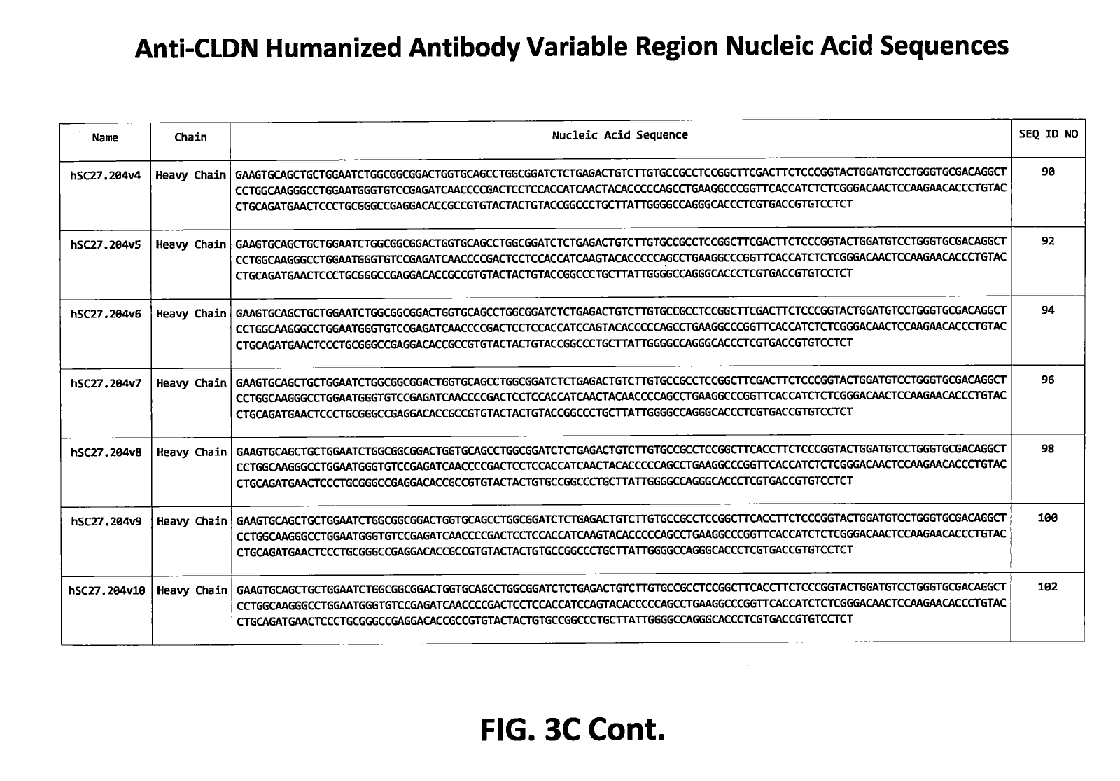

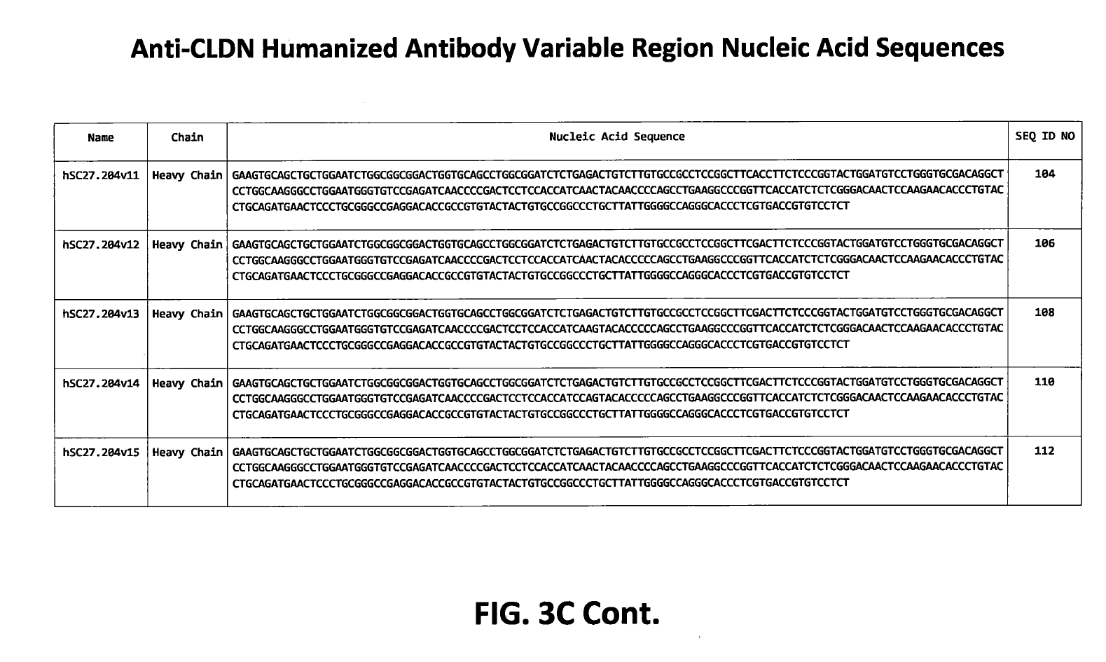

It will be appreciated that the annotated CDRs and framework sequences as provided in the appended FIGS. 3A and 3B are defined as per Kabat et al. using a proprietary Abysis database. However, as discussed herein one skilled in the art could readily identify CDRs in accordance with definitions provided by Chothia et al., ABM or MacCallum et al as well as Kabat et al. As such, anti-CLDN humanized antibodies comprising one or more CDRs derived according to any of the aforementioned systems are explicitly held to be within the scope of the instant invention.

b. Antibody Fragments, Derivatives or Constructs

In particularly preferred embodiments the CLDN binding domain will comprise an antibody fragment, derivative or construct. More particularly, regardless of which form of antibody (e.g. chimeric, humanized, etc.) is selected to practice the invention it will be appreciated that immunoreactive fragments of the same may be used, as part of a CLDN CAR, in accordance with the teachings herein. In a broad sense an “antibody fragment” comprises at least an immunoreactive portion of an intact antibody. That is, as used herein, the term “antibody fragment” includes at least an antigen-binding fragment or portion of an intact antibody and the term “antigen-binding fragment” refers to a polypeptide fragment of an immunoglobulin or antibody that immunospecifically binds or reacts with an immunogenic determinant of CLDN or competes with the intact antibody from which the fragments were derived for specific antigen binding. Moreover, for the purposes of the instant invention an “antibody construct” or “antibody derivative” shall be held to mean any molecular structure comprising an antibody fragment. Preferably such derivatives or constructs shall be non-natural and will be fabricated to impart beneficial molecular properties while maintaining the immunoreactive (or immunospecific) nature of the antibody.

Exemplary compatible antibody fragments, constructs or derivatives include: variable light chain fragments (VL), variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed or derived from antibody fragments. In other embodiments the CLDN binding domain of the instant invention may comprise an intact antibody, a scFv-Fc construct, a minibody, a diabody, a scFv construct, a Fab-scFv2 construct, a Fab-scFv construct or a peptibody. In certain aspects the CLDN binding domain will be covalently linked (e.g., by using art-recognized genetic engineering techniques) to the transmembrane and intracellular domains of the CAR. In other embodiments the CLDN binding domain may be non-covalently linked (e.g., via an Fc portion of the binding domain as set forth in U.S.P.N. 2015/0139943) to the transmembrane and intracellular domains of the CAR. Each form of binding domain attachment is compatible with the instant invention as long as the sensitized lymphocytes are able to induce the desired immune response.

In particularly preferred embodiments, and as shown in the appended Examples, the CLDN binding domain will comprise a scFv construct. As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain and light chain fragments are linked via a spacer sequence. Various methods for preparing an scFv are known, and include methods described in U.S. Pat. No. 4,694,778. Anti-CLDN scFv constructs compatible with the instant invention are described in more detail in the Examples appended hereto.

In other embodiments, the CLDN binding domain is one that comprises an Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such a binding domain may comprise an immunoreactive region linked to an Fc sequence comprising at least one free cysteine capable of conferring in vivo stability to the fragment. In other embodiments the Fc region may be modified using art-recognized techniques to modify the pharmacokinetics or pharmacodynamics of the disclosed CARs and sensitized lymphocytes.

Where the CLDN binding domain comprises an Fc portion it may be non-covalently linked or joined with the remaining portions of the CAR via an extracellular Fc receptor or binding molecule (“Fc binder”) that is operably associated with the transmembrane and intracellular domains. As used herein the term “Fc binder” is held to mean any molecule or portion thereof that binds to, or associates with, the Fc portion of an antibody (e.g., an Fc receptor). Such constructs (i.e., a “proto-CAR” comprising an Fc binder, transmembrane domain and intracellular signaling domain) may be fabricated using standard molecular biology techniques and associated with the selected lymphocytes (autologous or allogeneic) as described herein (e.g., via transduction) to generate “primed lymphocytes”. At some point prior to introduction into the patient, the primed lymphocytes may then be exposed to selected CLDN binding domain(s) comprising at least an Fc portion under conditions that allow association of the CLDN binding domain(s) with the proto-CAR. The non-covalent association of the binding domain with the proto-CAR provide the CLDN sensitized lymphocytes of the instant invention and may be used to inhibit tumorigenic cell proliferation as described herein (see generally U.S.P.N. 2015/0139943 which is incorporated herein in its entirety).

In those embodiments comprising a proto-CAR the Fc binder may comprise an Fc receptor such as an Fc-gamma receptor, an Fc-alpha receptor or an Fc-epsilon receptor. In certain selected embodiments the Fc receptor may comprise the ligand binding domain of CD16 (e.g., CD16A or CD16B), CD32 (e.g., CD32A or CD32B) or CD64 (e.g., CD64A, CD64B or CD64C). In certain other embodiments the Fc binder will not be an Fc receptor. For example the Fc binder may comprise all or part of protein A or protein G as long as the proto-CAR has the ability to associate with the CLDN binding domain. In other embodiments the Fc binder may comprise an immunoreactive antibody or fragment or construct or derivative thereof that binds the Fc portion of an immunoglobulin. As to such embodiments the Fc binder may, for example comprise an scFv, a nanobody or a minibody. Similarly CLDN binding domains compatible with such embodiments include any molecule that is capable of being bound by the Fc binder and immunospecifically reacting with CLDN. In some embodiments the CLDN binding domain will comprise intact CLDN monoclonal antibodies or mixtures of intact CLDN monoclonal antibodies. In other embodiments the CLDN binding domain may comprise intact polyclonal CLDN antibodies (preferably fully human). In yet other embodiments the CLDN binding domain may comprise a scFv-Fc construct. More generally, those of skill in the art will readily be able to identify proto-CAR compatible CLDN binding regions based on the teachings of the instant disclosure.

Moreover, as would be readily recognized by those skilled in the art, the disclosed fragments, construct or derivatives can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

c. Post-Production Selection

No matter how obtained, antibody-producing cells (e.g., hybridomas, yeast colonies, etc.) may be selected, cloned and further screened for desirable characteristics including, for example, high affinity for CLDN. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

4. Characteristics of CLDN Binding Domains

In selected embodiments, antibody-producing cells (e.g., hybridomas or yeast colonies) may be selected, cloned and further screened for favorable properties including, for example, robust growth, high antibody production and, as discussed in more detail below, desirable binding domain characteristics. In other cases characteristics of the antibody may be imparted by selecting a particular antigen (e.g., a specific CLDN domain) or immunoreactive fragment of the target antigen for inoculation of the animal. In still other embodiments the selected antibodies may be engineered as described above to enhance or refine immunochemical characteristics such as affinity or pharmacokinetics fragments.

a. Binding Domain Affinity

Disclosed herein are antibodies that have a high binding affinity for a specific determinant e.g. CLDN6. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≦10⁻⁷ M. The antibody specifically binds antigen with high affinity when the K_{D} is ≦5×10⁻⁹ M, and with very high affinity when the K_{D} is ≦5×10⁻¹⁰ M. In one embodiment of the invention, the antibody has a K_{D} of ≦10⁻⁹ M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≦2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M or less than 5×10⁻¹⁵ M.

In certain embodiments, an antibody of the invention that immunospecifically binds to a determinant e.g. CLDN may have an association rate constant or kₒₙ (or kₐ₎ rate (antibody+antigen (Ag)^{k}ₒₙ←antibody-Ag) of at least 10⁵ M⁻¹s⁻¹, at least 2×10⁵ M⁻¹s⁻¹, at least 5×10⁵ M⁻¹s⁻¹ , at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an antibody of the invention that immunospecifically binds to a determinant e.g. CLDN may have a disassociation rate constant or k_{off} (or k_{d)} rate (antibody+antigen (Ag)^{k}_{off}←antibody-Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

b. Binning and Epitope Mapping

As used herein, the term “binning” refers to methods used to group antibodies (or binding domains) into “bins” based on their antigen binding characteristics and whether they compete with each other. The initial determination of bins may be further refined and confirmed by epitope mapping and other techniques as described herein. However it will be appreciated that empirical assignment of antibodies to individual bins provides information that may be indicative of the therapeutic potential of the disclosed antibodies.

More specifically, one can determine whether a selected reference antibody (or fragment thereof) competes for binding with a second test antibody (i.e., is in the same bin) by using methods known in the art and set forth in the Examples herein. In one embodiment, a reference antibody is associated with CLDN antigen under saturating conditions and then the ability of a secondary or test antibody to bind to CLDN is determined using standard immunochemical techniques. If the test antibody is able to substantially bind to CLDN at the same time as the reference anti-CLDN antibody, then the secondary or test antibody binds to a different epitope than the primary or reference antibody. However, if the test antibody is not able to substantially bind to CLDN at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment being tested inhibits specific binding of a reference antibody to a common antigen. Typically, such an assay involves the use of purified antigen (e.g., CLDN or a domain or fragment thereof) bound to a solid surface or cells, an unlabeled test antibody and a labeled reference antibody. Competitive inhibition is measured by determining the amount of label bound to the solid surface or cells in the presence of the test antibody. Usually the test antibody is present in excess and/or allowed to bind first. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more.

Conversely, when the reference antibody is bound it will preferably inhibit binding of a subsequently added test antibody (i.e., a CLDN antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In preferred embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

“Surface plasmon resonance,” refers to an optical phenomenon that allows for the analysis of real-time specific interactions by detection of alterations in protein concentrations within a biosensor matrix.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In preferred embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which the antibodies in a bin bind. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds. The term “epitope” is used in its common biochemical sense and refers to that portion of the target antigen capable of being recognized and specifically bound by a particular antibody. In certain embodiments, epitopes or immunogenic determinants include chemically active surface groupings of molecules such as amino acids, sugar side chains, phosphoryl groups, or sulfonyl groups, and, in certain embodiments, may have specific three-dimensional structural characteristics, and/or specific charge characteristics. In certain embodiments, an antibody is said to specifically bind an antigen when it preferentially recognizes its target antigen in a complex mixture of proteins and/or macromolecules.

When the antigen is a polypeptide such as CLDN, epitopes may generally be formed from both contiguous amino acids and noncontiguous amino acids juxtaposed by tertiary folding of a protein (“conformational epitopes”). In such conformational epitopes the points of interaction occur across amino acid residues on the protein that are linearly separated from one another. Epitopes formed from contiguous amino acids (sometimes referred to as “linear” or “continuous” epitopes) are typically retained upon protein denaturing, whereas epitopes formed by tertiary folding are typically lost upon protein denaturing. An antibody epitope typically includes at least 3, and more usually, at least 5 or 8-10 amino acids in a unique spatial conformation. Methods of epitope determination or “epitope mapping” are well known in the art and may be used in conjunction with the instant disclosure to identify epitopes on CLDN bound by the disclosed antibodies.

Compatible epitope mapping techniques include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Other compatible methods comprise yeast display methods. In other embodiments Modification-Assisted Profiling (MAP), also known as Antigen Structure-based Antibody Profiling (ASAP) provides a method that categorizes large numbers of monoclonal antibodies directed against the same antigen according to the similarities of the binding profile of each antibody to chemically or enzymatically modified antigen surfaces (U.S.P.N. 2004/0101920). This technology allows rapid filtering of genetically identical antibodies, such that characterization can be focused on genetically distinct antibodies. It will be appreciated that MAP may be used to sort the CLDN antibodies of the invention into groups of antibodies binding different epitopes.

Once a desired epitope on an antigen is determined, it is possible to generate antibodies to that epitope, e.g., by immunizing with a peptide comprising the epitope using techniques described in the present invention. Alternatively, during the discovery process, the generation and characterization of antibodies may elucidate information about desirable epitopes located in specific domains or motifs. From this information, it is then possible to competitively screen antibodies for binding to the same epitope. An approach to achieve this is to conduct competition studies to find antibodies that compete for binding to the antigen. A high throughput process for binning antibodies based upon their cross-competition is described in WO 03/48731. Other methods of binning or domain level or epitope mapping comprising antibody competition or antigen fragment expression on yeast are well known in the art.

B. Optional Hinge Region

As used herein, the term “hinge region” refers to a flexible polypeptide connector region (also referred to herein as “hinge”) that may be included within the CAR ectodomain providing structural flexibility to flanking polypeptide regions. The hinge region may consist of natural or synthetic polypeptides. It will be appreciated by those skilled in the art that hinge regions may improve the function of the CAR by promoting optimal positioning of the antigen recognition domain in relationship to the portion of the antigen recognized by the antigen recognition domain. It will be appreciated that, in some embodiments, the hinge region may not be required for optimal CAR activity. In other embodiments a beneficial hinge region comprising a short sequence of amino acids promotes CAR activity by facilitating flexibility of the antigen binding domain or antibody. The sequence encoding the hinge region may be positioned between the antigen recognition moiety (e.g., an anti-CLDN scFv) and the transmembrane domains. The hinge sequence can be any moiety or sequence derived or obtained from any suitable molecule. In one embodiment, for example, the hinge sequence is derived from the human CD8a molecule or a CD28 molecule. A “hinge region” derived from an immunoglobulin (e.g., IgGI) is generally defined as stretching from Glu216 to Pro230 of human IgG1. Hinge regions of other IgG isotypes may be aligned with the IgG1 sequence by placing the first and last cysteine residues forming inter-heavy chain disulfide (S—S) bonds in the same positions. In other embodiments the hinge region may be of natural occurrence or non-natural occurrence, including but not limited to an altered hinge region as described in U.S. Pat. No. 5,677,425. Of course, when certain binding domains such as (Fab′)₂ or an intact antibody are used in the CAR it will naturally follow that the corresponding hinge region will be included.

In other selected embodiments the hinge region can include complete hinge region derived from an antibody of a different class or subclass from that of the CH1 domain. The term “hinge region” can also include regions derived from human CD8α molecule or a 0028 molecule and any other receptors that provide a similar function in providing flexibility to flanking regions. The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Those skilled in the art will appreciate that compatible hinge regions are well known and, as such, operable embodiments may readily be selected and incorporated in the CLDN CARs of the instant invention.

C. Transmembrane/Spacer Domain

As alluded to above, the CLDN CARs of the instant invention preferably comprise a transmembrane domain that is interposed between the extracellular CLDN binding domain and/or hinge region, and the intracellular or cytoplasmic signaling domain. For the purposes of the instant discussion the term “transmembrane domain” will be used with the understanding that while it always includes amino acid residues that are physically buried in the lipid bilayer of a cellular membrane, it may include support or “spacer domains” that can extend beyond either side of the cell membrane. Those of skill in the art can readily distinguish between the functional aspects of the CAR components and easily determine what constitutes a compatible transmembrane domain in view of the instant disclosure.

It will be appreciated that the transmembrane domain may be derived from a natural polypeptide, or may be artificially designed. Compatible transmembrane domains may be derived from any membrane-binding or transmembrane protein which may be modified or truncated as necessary. For example, transmembrane domains derived from a T cell receptor a or β chain, a Fc region of an IgG (such as IgG4), a CD3ζ chain, CD28, CD3ε, CD45, CD4, CD5, CD8, CD9, CD16, CD22, CD33, CD37, CD64, CD80, CD86, CD134, CD137, ICOS, CD154 or a GITR are all compatible with various embodiments of the disclosed CLDN CAR constructs. In certain embodiments it is preferred to employ the transmembrane domain of CD8ζ, FcRη, FcεR1-γ and -β, MB1 (Igα), B29 or CD3-γ, ζ, or ε, in order to retain physical association with other members of the receptor complex. Compatible artificial transmembrane domains may comprise various polypeptide sequences incorporating high levels of hydrophobic residues such as leucine and valine. In other preferred embodiments the transmembrane domain may comprise a triplet of phenylalanine, tryptophan and valine which is located at each end of the synthetic transmembrane domain.

Certain embodiments of the invention will comprise transmembrane domains having a spacer. In the CLDN CARs of the present invention, a “spacer domain” or “spacer region” is an amino acid sequence that can be arranged between an extracellular functional domain (e.g., the antigen binding domain or the hinge region if included) and the transmembrane domain, or between the intracellular signaling domain and the transmembrane domain. The spacer domain means any oligopeptide or polypeptide that serves to link the transmembrane domain with the extracellular domain and/or the transmembrane domain with the intracellular domain, with the intent to optimally position these elements within the CAR polypeptide for efficient CAR function. The spacer domain comprises up to 300 amino acids, preferably 10 to 100 amino acids, and most preferably 25 to 50 amino acids. The spacer domain preferably has a sequence that promotes binding of the CLDN CAR with CLDN and enhances transmembrane signaling into a cell. Examples of amino acids that are expected to promote the binding include cysteine, a charged amino acid, and serine and threonine in a potential glycosylation site, and these amino acids can be used as an amino acid constituting the spacer domain. In preferred embodiments the spacer may comprise all or part of an antibody constant region (e.g., IgG1 CH or CL) which may optionally dimerize.

Other compatible spacers include glycine polymers (G)ₙ, glycine-serine polymers, glycine-alanine polymers, alanine-serine polymers, and other flexible spacers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains.

Those skilled in the art will appreciate that compatible transmembrane domains are well known in the art and, as such, operable embodiments may readily be selected and incorporated in the CLDN CARs of the instant invention.

D. Intracellular Signaling Domain

In addition to the extracellular CLDN binding domain and the transmembrane domain, the CLDN CARs of the instant invention will incorporate an intracellular or cytoplasmic domain comprising at least one signaling and/or T cell activating moiety. The intracellular signaling domain used in the present invention is a molecule that can transmit one or more signals into a cell when the extracellular domain present (or non-covalently associated) within the same molecule binds to (interacts with) CLDN. The binding of CLDN triggers a signal that passes along the CAR and is transmitted intracelliularly to activate the sensitized lymphocyte. This lymphocyte activation triggers the desired immune response that results in the elimination of the target cell.

The two signal theory of T-lymphocyte activation proposes that two signals are required to efficiently activate T-cells: first, antigenic peptides presented in the context of an MHC molecule interact with the alpha:beta chain heterodimer of the TCR, leading to conformational changes that result in activation of a signal from the cytoplasmic domains found in protein components of the TCR complex; and second, transmission of a signal from the cytoplasmic domain of a single or several costimulatory molecules as they interact with their cognate ligands on the cell presenting the peptide:MHC complex. More specifically it is known that a signal generated only via a TCR complex may be insufficient to activate a T cell, and a secondary or costimulating signal is also required to avoid a state of T-cell inactivity known an anergy. Natural T cell-activation is transmitted by two different kinds of cytoplasmic signaling sequences, that is, a sequence for initiating antigen-dependent primary activation via a TCR complex (primary cytoplasmic signaling sequence) and a sequence for acting antigen-independently to provide a secondary or costimulating signal (secondary cytoplasmic signaling sequence). In a preferable aspect, the CLDN CAR of the present invention comprises the primary cytoplasmic signaling sequence and/or the secondary cytoplasmic signaling sequence as the CAR endodomain.

In general, signaling motifs found in the cytoplasmic domains of immune system receptors maybe activating or inhibitory. The primary cytoplasmic signaling sequence that stimulates the activation may comprise a signal transduction motif known as an immunoreceptor tyrosine-based activation motif (ITAM) [Nature, vol. 338, pp. 383-384 (1989)]. On the other hand, the primary cytoplasmic signaling sequence that acts in an inhibitory way comprises a signal transduction motif known as an immunoreceptor tyrosine-based inhibition motif (ITIM). In the present invention, an intracellular domain having an ITAM or an ITIM can be used.

The primary cytoplasmic signaling sequence that transmits the first stimulating signal for T-cell activation from the native TCR complex is an ITAM found in the CD3ζ chain, but it is known that other ITAMs may also be employed to transmit positive primary activating signal. Examples of the intracellular domain having an ITAM that can be used in the present invention include intracellular domains having ITAM derived from CD3ζ, FcRγ, FcRβ, CD3γ, CD3 δ, CD3ε, CD5, CD22, CD79a, CD79b, and CD66d. Specifically, examples of the ITAM include peptides having sequences of amino acid numbers 51 to 164 of CD3ζ (NCBI RefSeq: NP_932170.1), amino acid numbers 45 to 86 of FcεRIγ (NCBI RefSeq: NP_004097.1), amino acid numbers 201 to 244 of FcεRIβ (NCBI RefSeq: NP_000130.1), amino acid numbers 139 to 182 of CD3γ (NCBI RefSeq: NP_000064.1), amino acid numbers 128 to 171 of CD3 δ (NCBI RefSeq: NP_000723.1), amino acid numbers 153 to 207 of CD3ε (NCBI RefSeq: NP_000724.1), amino acid numbers 402 to 495 of CD5 (NCBI RefSeq: NP_055022.2), amino acid numbers 707 to 847 of 0022 (NCBI RefSeq: NP_001762.2), amino acid numbers 166 to 226 of CD79a (NCBI RefSeq: NP_001774.1), amino acid numbers 182 to 229 of CD79b (NCBI RefSeq: NP_000617.1), and amino acid numbers 177 to 252 of CD66d (NCBI RefSeq: NP_001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein.

The secondary, costimulatory signal may come from the cytoplasmic domain of a variety of co-stimulatory molecules, the best characterized of which is CD28. CD28 is expressed on T-cells and is the receptor for CD80 (B7.1) and CD86 (B7.2). However, other co-stimulatory molecules include, but are not limited to the CD27 molecule, the CD137/4-1BB molecule, the CD134/OX40 molecule, and other intracellular signaling molecules known in the art. CD134/OX40 is known to enhance T-cell clonal expansion, likely by suppressing apoptosis, and may play a role in the establishment of memory cells. 4-1BB, also known as CD137, transmits a potent costimulatory signal to T-cells, promoting differentiation and enhancing long-term survival of T lymphocytes. As each of these costimulatory molecules activates different intracellular signaling pathways and may have differing effects in different populations of T-lymphocytes, domains from, one, several, or each may be included in the endodomain of the CAR in order to maximize T-cell activation and other desired properties of the CAR. In a preferred embodiment, the CD28, CD27, 4-1 BB, and OX40 molecules are human. Examples of the intracellular domain comprising a secondary cytoplasmic signaling sequence that can be used in the present invention include sequences derived from CD2, CD4, CD5, CD8α, CD8β, CD28, CD134, CD137, ICOS, and CD154. Specific examples thereof include peptides having sequences of amino acid numbers 236 to 351 of CD2 (NCBI RefSeq: NP-001758.2), amino acid numbers 421 to 458 of CD4 (NCBI RefSeq: NP-000607.1), amino acid numbers 402 to 495 of CD5 (NCBI RefSeq: NP-055022.2), amino acid numbers 207 to 235 of CD8α (NCBI RefSeq: NP-001759.3), amino add numbers 196 to 210 of CD83 (GenBank: AAA35664.1), amino acid numbers 181 to 220 (SEQ ID No.: 25) of CD28 (NCBI RefSeq: NP-006130.1), amino acid numbers 214 to 255 of CD137 (4-1BB, NCBI RefSeq: NP-001552.2), amino acid numbers 241 to 277 of CD134 (OX40, NCBI RefSeq: NP-003318.1), and amino acid numbers 166 to 199 of ICOS (NCBI RefSeq: NP-036224.1), and their variants having the same function as these peptides have.

The signaling/activating domain(s) of the CLDN CAR encoded by the disclosed nucleic acid sequence can comprise any one of aforementioned signaling domains and any one or more of the aforementioned intercellular T-cell activating domains in any combination. For example, the inventive nucleic acid sequence can encode a CAR comprising a CD28 signaling domain and intracellular T-cell activating domains of CD28 and CD3ζ. Alternatively, for example, the nucleic acid sequences of the invention can encode a CAR comprising a CD8α signaling domain and T cell signaling domains of CD28, CD3ζ, the Fc receptor gamma (FcRγ) chain, and/or 4-1BB.

Those skilled in the art will appreciate that each of the aforementioned signaling/stimulatory domains are compatible with the instant invention and may be used effectively (alone or preferably in combination) with the disclosed CLDN CARS. Accordingly, each of the aforementioned moieties, in any combination or configuration are expressly contemplated as being within the scope of the instant invention as components of the intracellular/cytoplasmic domain.

### V. CAR Nucleic Acids and Vectors

The invention provides an isolated or purified nucleic acid sequence encoding an anti-CLDN chimeric antigen receptor, wherein the CAR preferably comprises an extracellular binding domain (e.g., a scFv), a transmembrane domain and an intracellular signaling domain (e.g., a T-cell activation moiety). As used herein “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides.

By “isolated” is meant the removal of a nucleic acid from its natural environment. By “purified” is meant that a given nucleic acid, whether one that has been removed from nature (including genomic DNA and mRNA) or synthesized (including cDNA) and/or amplified under laboratory conditions, has been increased in purity, wherein “purity” is a relative term, not “absolute purity.” It is to be understood, however, that nucleic acids and proteins may be formulated with diluents or adjuvants and still for practical purposes be isolated. For example, nucleic adds typically are mixed with an acceptable carrier or diluent when used for introduction into cells.

As described herein and shown in the appended Examples, nucleic add sequences compatible with the invention can be generated using methods known in the art. For example, nucleic add sequences, polypeptides, and proteins can be recombinantly produced using standard recombinant DNA methodology (see, e.g., Sambrook et al., Molecular Cloning: A Laboratory Manual, 3rd ed., Cold Spring Harbor Press, Cold Spring Harbor, N.Y. 2001). Further, a synthetically produced nucleic add sequence encoding the CLDN CAR can be isolated and/or purified from a source, such as a CHO cell, plant, a bacterium, an insect, or a mammal, e.g., a rat, a human, etc. Methods of isolation and purification are well known in the art. Alternatively, the nucleic add sequences described herein can be commercially synthesized. In this respect, the inventive nucleic acid sequence can be synthetic, recombinant, isolated, and/or purified.

A nucleic acid sequence of the invention can encode a CLDN CAR of any length, i.e., the CAR can comprise any number of amino adds, provided that the CAR retains its biological activity, e.g., the ability to specifically bind to antigen and treat or prevent disease in a mammal, etc. For example, the CAR can comprise 50 or more, 60 or more, 100 or more, 250 or more, or 500 or more amino adds. Preferably, the CAR is about 50 to about 700 amino adds (e.g., about 70, about 80, about 90, about 150, about 200, about 300, about 400, about 550, or about 650 amino adds), about 100 to about 500 amino adds (e.g., about 125, about 175, about 225, about 250, about 275, about 325, about 350, about 375, about 425, about 450, or about 475 amino adds), or a range defined by any two of the foregoing values.

Included in the scope of the invention are nucleic add sequences that encode functional portions of the CLDN CAR described herein. The term “functional portion,” when used in reference to a CAR, refers to any part or fragment of the CAR of the invention, which part or fragment retains the biological activity of the CAR of which it is a part (the parent CAR). Functional portions encompass, for example, those parts of a CAR that retain the ability to recognize target cells or provide an immunomodulatory signal, or treat a disease, to a similar extent, the same extent, or to a higher extent, as the parent CAR. In reference to a nucleic add sequence encoding the parent CLDN CAR, a nucleic add sequence encoding a functional portion of the CAR can encode a protein comprising, for example, about 10%, 25%, 30%, 50%, 68%, 80%, 90%, 95%, or more, of the parent CAR. In this regard compatible nucleic acid sequences can encode a functional portion of a CAR that contains additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The invention also provides nucleic acid sequences encoding functional variants of the CLDN CAR. The term “functional variant,” as used herein, refers to a CAR, a polypeptide, or a protein having substantial or significant sequence identity or similarity to the CAR encoded by the disclosed nucleic acid sequences, which functional variant retains the CLDN binding capacity of the CAR of which it is a variant. Functional variants encompass, for example, those variants of the CAR described herein (the parent CAR) that retain the ability to recognize CLDN positive target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR.

Regardless of the precise form of the CLDN CAR it will be appreciated that the nucleic adds of the present invention may be used for ex vivo transformation of selected host cells (e.g., lymphocytes) or introduced directly into the subject for in vivo gene therapy. In each case the disclosed nucleic adds may be combined with a substance that promotes transference of a nucleic add into a cell, for example, a reagent for introducing a nucleic add such as a liposorne or a cationic lipid, in addition to other excipients disclosed herein. In certain preferred embodiments the nucleic adds of the instant invention will be combined with, or integrated into, a vector is suitable for in vivo gene therapy.

Accordingly, in conjunction with the foregoing the present invention provides compositions comprising CLDN CAR nucleic adds which, together with a pharmaceutically acceptable carrier, may be used as an active ingredient or to generate sensitized lymphocytes. Suitable pharmaceutically acceptable additives are well known to a person skilled in the art. Examples of the pharmaceutically acceptable additives or excipients include phosphate buffered saline (e.g. 0.01 M phosphate, 0.138 M NaCl, 0.0027 M KCl, pH 7.4), an aqueous solution containing a mineral acid salt such as a hydrochloride., a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. Compositions of the present invention can be formulated into a known form suitable for parenteral administration, for example, injection or infusion. Further, such compositions may comprise formulation additives such as a suspending agent, a preservative, a stabilizer and/or a dispersant, and a preservation agent for extending a validity term during storage. Further the composition may be in a dry form for reconstitution with an appropriate sterile liquid prior to use.

In addition to the nucleic acid sequence encoding the CLDN CAR, compatible vectors preferably comprise expression control sequences, such as promoters, enhancers, polyadenylation signals, transcription terminators, internal ribosome entry sites (IRES), and the like, that provide for the expression of the nucleic add sequence in a host cell. In this regard a large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system, the Ecdysone inducible system, the T-REX ™ system (Invitrogen, Carlsbad, Calif.), LACSWITCH™ System (Stratagene, San Diego, Calif.), and the Cre-ERT tamoxifen inducible recombinase system. In addition the CLDN CAR may be associated with a gene that can be a marker for confirming expression of the nucleic add (e.g. a drug resistance gene, a gene encoding a reporter enzyme, or a gene encoding a fluorescent protein).

In certain embodiments the nucleic add encoding the CLDN CAR, along with any control elements, can preferably be inserted into a vector that can then be introduced into a selected cell to provide the disclosed CLDN sensitized lymphocytes. In preferred embodiments the vector can be, for example, a plasmid, a transposon, a cosmid or a viral vector (e.g., phage, retroviral, lentiviral or adenoviral). For example, a virus vector such as a retrovirus vector (including an oncoretrovirus vector, a lentivirus vector, and a pseudo type vector), an adenovirus vecto, an adeno-associated virus (AAV) vector, a simian virus vector, a vaccinia virus vector or a sendai virus vector, an Epstein-Barr virus (EBV) vector, and a HSV vector can be used.

More generally the terms “vector”, “cloning vector” and “expression vector” mean the vehicle by which a DNA or RNA sequence (e.g., a foreign gene encoding a CLDN CAR) can be introduced into a host cell, so as to transform the host and promote expression (e.g. transcription and translation) of the introduced sequence. It will be appreciated that the introduced gene or sequence may include regulatory or control sequences, such as start, stop, promoter, signal, secretion or other sequences used by a cell's genetic machinery. As described herein compatible vectors are well known in the art and include plasmids, transposons, phages, viruses, etc. The vector may then be used to transform the selected lymphocytes (autologous or allogeneic) to provide the disclosed sensitized lymphocytes. For the purposes of the instant disclosure the term “transform” or “transformation” will be used in its most general sense and shall be held to mean the introduction of a heterologous gene, DNA or RNA sequence to a host cell (prokaryotic or eukaryotic), so that the host cell will express the introduced gene or sequence to produce a desired substance, typically a protein or enzyme coded by the introduced gene or sequence. Exemplary methods of cell transformation compatible with the invention comprise transfection and transduction. As used herein the term “transfection” means the introduction of a foreign nucleic acid or gene into a cell (prokaryotic or eukaryotic) using physical or chemical means while the term “transduction” means the introduction of a foreign nucleic acid or gene into a cell (prokaryotic or eukaryotic) through use of a viral vector.

In terms of transduction phage or viral vectors can be introduced into host cells, preferably after growth of infectious particles in suitable packaging cells, many of which are commercially available. Compatible transduction methods and packaging cells are set forth in the Examples below and would be readily discernable to the skilled artisan in view of the instant disclosure.

By way of example, when a retrovirus vector is to be used, compositions compatible with the teachings herein can be generated by selecting a suitable packaging cell based on a LTR sequence and a packaging signal sequence possessed by the vector and preparing a retrovirus particle using the packaging cell. Examples of the packaging cell include PG13 (ATCC CRL-10686), PA317 (ATCC CRL-9078), GP+E-86 and G+envAm-12, and Psi-Grip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies. Similar systems are also commercially available for the fabrication of compatible lentiviral vectors in accordance with the teachings herein. Such vectors may be used to transduce selected lymphocyte populations to provide the desired CLDN sensitized lymphocytes.

In addition, non-viral packaging vector systems can also be used in the present invention in combination with a liposome and a condensing agent such as a cationic lipid as described in WO 96/10038, WO 97/18185, WO 97/25329, WO 97/30170 and WO 97/31934 (which are incorporated herein by reference).

Similarly, many methods of transfection are compatible with the instant invention and may be used in conjunction with the teachings herein to provide the desired compositions. As discussed, transfection typically refers to the introduction of one or more exogenous polynucleotides into a host cell by using physical or chemical methods. Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Additionally, electroporation, sonoporation, impalefection, optical transfection and hydro dynamic delivery comprise some non-chemical based gene transfection methods compatible with the instant invention.

Regardless of which methodology is selected to effect transformation, it will be appreciated that the CLDN CAR nucleic acid constructs and vectors may be used to generate the disclosed sensitized lymphocytes.

### VI. Host Cells

A vector comprising a nucleic acid encoding the CLDN CAR can be introduced into any host cell that is capable of carrying and/or expressing the CAR protein, including any suitable prokaryotic or eukaryotic cell. Compatible methods of transformation comprise the use of lentiviral, and retroviral systems along with transposons and naked RNA. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell (e.g., plant, animal, fungi, or algae), a prokaryotic cell (e.g., bacteria or protozoa) or a viral or retroviral vector. The host cell can be a cultured or “off-the-shelf” cell or a primary cell (i.e., isolated directly from a subject). The host cell can be an adherent cell or a suspended cell, i.e., a cell that grows in suspension. Suitable host cells are known in the art and include, for instance, DH5 α E. coil cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell can be a mammalian cell. The host cell preferably is a human cell. The host cell can be of any cell type, can originate from any type of tissue, and can be of any developmental stage. For example, a cell collected, isolated, purified or induced from a body fluid, a tissue or an organ such as blood (peripheral blood, umbilical cord blood etc.) or bone marrow can be used. A peripheral blood mononuclear cell (PBMC), an immune cell [a dendritic cell, a B cell, a hematopoietic stem cell, a macrophage, a monocyte, a NK cell or a hematopoietic cell (a neutrophil, a basophil)], an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In particularly preferred embodiments the host cell can be a peripheral blood lymphocyte (PBL), a peripheral blood mononuclear cell (PBMC), or a natural killer (NK) cell. Preferably, the host cell comprises a natural killer (NK) cell. In other preferred embodiments the host cell will be a T-cell and, in selected embodiments, a cytotoxic T-cell. Methods for selecting suitable mammalian host cells and methods for transformation, culture, amplification, screening, and purification of cells are known in the art.

The invention provides an isolated host cell that expresses nucleic acid sequence encoding the CLDN CARs described herein or compositions of the same. In particularly preferred embodiments the host cell comprises a lymphocyte which is transformed into a CLDN sensitized lymphocyte upon expression of the disclosed CARs. In one embodiment, the host cell is a T-cell. The T-cell of the invention can be any T-cell, such as a cultured T-cell (e.g., a primary T-cell, or a T-cell from a cultured T-cell line, or a T-cell obtained from a mammal). If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th1 and Th2 cells, a CD8+ T-cell (e.g., a cytotoxic T-cell), a tumor infiltrating cell, a memory T-cell, a naïve T-cell, and the Ike. In one embodiment, the T-cell is a CD8+ T-cell or a CD4+ T-cell. T-cell lines are available from commercial sources (e.g., the American Type Culture Collection and the German Collection of Microorganisms and Cell Cultures) and include, for example, Jurkat cells (ATCC TIB-152), Sup-T1 cells (ATCC CRL-1942), RPM8402 cells (DSMZ ACC-290), Karpas 45 cells (DSMZ ACC-545), and derivatives thereof.

In another embodiment, the host cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 5th ed., Janeway et al., eds., Garland Publishing, New York, N.Y. (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell Ones are available from commercial sources (e.g., the American Type Culture Collection) and include, for example, NK-92 cells (ATCC CRL-2407), NK92MI cells (ATCC CRL-2408), and derivatives thereof.

In autologous adoptive immunotherapy, a patient's circulating lymphocytes, or tumor infiltrated lymphocytes, are isolated (e.g., by apheresis) in vitro, preferably activated or stimulated by lymphokines such as IL-2 and then transduced with nucleic adds encoding a CLDN CAR construct. Following transduction the autologous sensitized lymphocytes are preferably expanded using cytokine support as known in the art and readministered to the patient. To achieve this, one would administer to an animal, or human patient, an immunologically effective amount of activated lymphocytes genetically modified to express a CLDN CAR gene as described herein. In such autologous procedures the activated lymphocytes (i.e., CLDN sensitized lymphocytes) are the patient's own cells that most preferably were earlier isolated from a blood or tumor sample and activated and expanded in vitro. In some aspects of the present invention T lymphocytes or NK cells from a patient having cancer would be isolated and transduced with the SCT1-h27.xx polynucleotide (Example 9 below) so that the CLDN CAR is expressed on the cell surface of the T cell or NK cell. The modified cells would then be readministered into the patient to target and kill tumor cells (see generally FIG. 7).

Other preferred aspects of the invention comprise allogeneic transplants of CLDN sensitized lymphocytes. In such embodiments the disclosed CLDN CARs may be introduced (e.g., through transduction) into lymphocytes obtained from a source other than the subject to be treated. Some aspects of the instant invention comprise the use of allogeneic lymphocytes obtained from a donor that has been immunologically matched with the recipient to reduce the chance of rejection. In other aspects the disclosed CARs will be introduced into “off-the-shelf” allogeneic lymphocytes (see PMID: 26183927 which is incorporated herein by reference) that have been modified to facilitate transplantation and generate the appropriate immune response upon contact with the target cell. It will be appreciated that the use of such prefabricated allogeneic lymphocyte preparations may provide several advantages in terms of preparing the pharmaceutically active sensitized lymphocytes and reducing the chances of patient rejection.

It will also be appreciated that the CLDN sensitized lymphocytes cells can be expanded in vitro before or after transformation with the CLDN CAR. Methods for expanding the selected cell populations are well known in the art and several commercial kits compatible with the instant invention are available. In this regard T cells and or NK cells may be expanded in vitro to provide more robust dosing options. For example, in accordance with the present invention NK cells may be preferentially expanded by exposure to cells that lack or poorly express major histocompatibility complex I and/or II molecules and which have been genetically modified to express membrane bound IL-15 and 4-1BB ligand (CDI37L), Such cell lines include, but are not necessarily limited to, K562 (ATCC, CCL 243), and the Wilms tumor cell line HFWT, the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastated neuroblastoma cell line TNB 1. Preferably the cell line used lacks or poorly expresses both MHC I and II molecules, such as the K562 and HFWT cell lines. Similar techniques allow for the expansion of selected T cell populations. In this regard some processes employ anti-CD3 plus autologous or allogeneic feeder cells and high doses of IL-2. Other processes use IL-7, II-15, IL-21 or combinations thereof for expansion and stimulation of T cells. It will be appreciated that each of the aforementioned processes, along with any process that provides the desired number of CLDN sensitized lymphocytes, is compatible with the instant invention.

### VII. Formulation and Administration of CLDN Sensitized Lymphocytes

As set forth herein the CLDN sensitized lymphocytes can be expanded in vitro for use in adoptive cellular immunotherapy comprising autologous or allogeneic lymphocytes. In this regard the compositions and methods of this invention can be used to generate a population of sensitized lymphocytes that preferably deliver both primary and costimulatory signals for use in the treatment of cancer and, by way of example, the treatment of lung cancer including small cell lung cancer, melanoma, breast cancer, prostate cancer, colon cancer, renal cell carcinoma, ovarian cancer, neuroblastoma, rhabdomyosarcoma, leukemia and lymphoma. The compositions and methods described in the present invention may be used in conjunction with other types of therapy for cancer, such as chemotherapy, surgery, radiation, gene therapy, and so forth.

The CLDN sensitized lymphocytes or host cells are preferably administered to a subject in the form of a pharmaceutical composition comprising one or more pharmaceutically acceptable carriers. In particularly preferred embodiments the disclosed pharmaceutical compositions will comprise a population of T cells or NK cells (autologous or allogeneic) that express the CLDN CAR. Besides such host cells pharmaceutical compositions of the invention can comprise other pharmaceutically active agents or drugs, such as chemotherapeutic agents (e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.) or adjuvant therapies that further stimulate the immune response. In a preferred embodiment, the pharmaceutical composition comprises isolated T cells or NK cells which express the disclosed CLDN CARs and more preferably a population of sensitized T cells or NK cells which express the disclosed CLDN CARs. In addition such compositions may comprise a pharmaceutically acceptable buffers, preservatives, excipients, etc. as is well known in the art.

Alternatively, nucleic acid sequences encoding the CLDN CAR, or vectors comprising a CLDN CAR-encoding nucleic acid sequence, can be formulated into a pharmaceutical composition and used to transduce lymphocytes ex vivo or administered directly to the patient. In such embodiments vector systems comprising viral vector host cells (e.g., lentiviral systems or retroviral systems) or directed artificial viral envelopes are preferred. Such vectors allow for the in vivo generation of CLDN sensitized lymphocytes which can then induce the desired anti-tumor immune response.

In any event the CLDN CAR host cells of the invention and any co-reagents can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al.(2000) Handbook of Pharmaceutical Excipients, 3^{rd} ed., Pharmaceutical Press.)

Suitable pharmaceutically acceptable carriers typically comprise substances that are relatively inert and can facilitate administration of the sensitized lymphocyte or host cell or can aid processing of the same into preparations that are pharmaceutically optimized for delivery to the site of action. Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Sensitized lymphocytes for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are well known in the art.

Formulations suitable for parenteral administration of CLDN sensitized lymphocytes (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include sodium chloride injection, Ringer's Solution, or Lactated Ringer's Injection.

Methods of introducing cellular components are also known in the art and include procedures such as those exemplified in U.S. Pat. Nos. 4,844,893 and 4,690,915. The amount of CLDN sensitized lymphocytes (e.g., T cells or NK cells) used can vary between in vitro and in vivo uses, as well as with the amount and type of the target cells. The amount administered will also vary depending on the condition of the patient and should be determined by the practitioner after considering all appropriate factors.

The particular dosage regimen of CLDN sensitized lymphocytes, i.e., dose, timing and repetition, will depend on the particular individual, as well as empirical considerations such as pharmacokinetics (e.g., half-life, clearance rate, etc.). For example, individuals may be given incremental dosages of sensitized lymphocytes produced as described herein. In selected embodiments the dosage may be gradually increased or reduced or attenuated based respectively on empirically determined or observed side effects or toxicity. Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSMA) or a CLDN antigen identified herein; reduction in the number of proliferative or tumorigenic cells, maintenance of the reduction of such neoplastic cells; reduction of the proliferation of neoplastic cells; or delay in the development of metastasis.

In view of the instant disclosure the CLDN CAR may be administered on a specific schedule. Generally, an effective dose of the sensitized lymphocytes is administered to a subject one or more times. More particularly, an effective dose of the CLDN CAR is administered to the subject once a month, more than once a month, or less than once a month. In certain embodiments, the effective dose of the CLDN sensitized lymphocytes may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the CLDN sensitized lymphocytes.

In certain preferred embodiments the course of treatment involving CLDN CAR will comprise multiple doses of the selected sensitized lymphocytes over a period of weeks or months. More specifically, CLDN sensitized lymphocytes of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

A typical amount of host cells administered to a mammal (e.g., a human) can be, for example, in the range of one million to 100 billion cells; however, amounts below or above this exemplary range are within the scope of the invention. For example, the daily dose of inventive host cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values). In preferred embodiments about 0.5 billion, 1.0 billion, 1.5 billion, 2 billion, 2.5 billion, 3 billion, 3.5 billion, 4 billion, 4.5 billion, 5 billion, 5.5 billion, 6 billion, 6.5 billion, 7 billion, 7.5 billion, 8 billion, 8.5 billion, 9 billion, 9.5 billion or 10 billion cells are administered to the patient in one or more doses.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

As discussed above compositions comprising sensitized the host cells expressing the CLDN CAR can be administered to a mammal using standard administration techniques, including intravenous, intraperitoneal, subcutaneous, pulmonary, transdermal, intramuscular or intranasal. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Moreover host cells expressing the CLDN CAR nucleic acid sequence, or a vector comprising the CAR-encoding nucleic acid sequence, can be administered with one or more additional therapeutic agents, which can be coadministered to the mammal. By “coadministering” is meant administering one or more additional therapeutic agents and the composition comprising the inventive host cells or the inventive vector sufficiently close in time such that the CLDN CAR can enhance the effect of one or more additional therapeutic agents, or vice versa. In this regard, the composition comprising the sensitized lymphocytes can be administered first, and the one or more additional therapeutic agents can be administered second, or vice versa. Alternatively, the composition comprising the CLDN sensitized lymphocytes and the one or more additional therapeutic agents can be administered simultaneously.

In selected preferred embodiments the CLDN sensitized lymphocytes will be administered in conjunction with lymphotoxic therapies to increase the availability of homeostatic cytokines (e.g., IL-7, IL-15, etc.) to support T cell expansion. In such protocols the lymphotoxic therapy will preferably be conducted prior to administration of the sensitized lymphocytes. More specifically it is believed that a lymphodepleting preparative regimen may enhance the efficacy of adoptive cell therapy by reducing endogenous lymphocytes thereby leading to the accumulation of homeostatic cytokines that support expansion and persistence of the administered sensitized lymphocytes. Further, such preparative treatments may lead to a transient reduction in the number and frequency of Tregs thereby diminishing lymphocyte suppression and an induction of gut damage which may lead to the systemic release of bacterial byproducts (e.g., lipopolysaccharides) that activate the innate immune system. Taken together such mechanisms can substantially enhance the receptiveness of the immune environment for the transplanted CLDN sensitized lymphocytes thereby promoting expansion and persistence of the same.

### VIII. Indications

The present invention provides for the use of CLDN sensitized lymphocytes of the invention for the treatment, maintenance and/or prophylaxis of various disorders including neoplastic, inflammatory, angiogenic and immunologic CLDN associated disorders. Preferred targets for treatment are neoplastic conditions comprising solid tumors and hematologic malignancies. In certain embodiments the CLDN CAR treatments of the invention will be used to inhibit, reduce or eliminate tumors or tumorigenic cells expressing CLDN. Preferably the “subject” or “patient” to be treated will be human although, as used herein, the terms are expressly held to comprise any mammalian species.

Neoplastic conditions subject to treatment in accordance with the instant invention may be benign or malignant; solid tumors or other blood neoplasia; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer including triple negative breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In particularly preferred embodiments the subject will be suffering from ovarian, pancreatic cancer, colorectal cancer, small cell lung cancer, non-small cell lung cancer, and gastric cancer. In the preferred embodiments the subject will be refractory as to ovarian cancer, pancreatic cancer, colorectal cancer, small cell lung cancer, non-small cell lung cancer, and gastric cancer.

In other preferred embodiments the disclosed CLDN CAR treatments are especially effective at treating lung cancer, including the following subtypes: small cell lung cancer and non-small cell lung cancer (e.g. squamous cell non-small cell lung cancer or squamous cell small cell lung cancer). In selected embodiments the CLDN sensitive lymphocytes can be administered to patients exhibiting limited stage disease or extensive stage disease. In other preferred embodiments the disclosed cellular compositions will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel).

In another particularly preferred embodiment the disclosed CLDN CAR treatments are effective at treating ovarian cancer, including ovarian-serous carcinoma and ovarian-papillary serous carcinoma.

In another preferred embodiment the CLDN CAR treatments of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed CLDN CAR treatments one or more times even though there is little or no indication of disease using standard diagnostic procedures. In some embodiments, the modulators will be administered on a regular schedule over a period of time, such as weekly, every two weeks, monthly, every six weeks, every two months, every three months every six months or annually. Given the teachings herein, one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the CLDN CAR treatments of the present invention may be used to prophylactically or as an adjuvant therapy to prevent or reduce the possibility of tumor metastasis following a debulking procedure. As used in the instant disclosure a “debulking procedure” is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed CLDN CAR treatments may be administered as suggested by clinical, diagnostic or theragnostic procedures to reduce tumor metastasis. The CLDN sensitized lymphocytes may be administered one or more times at pharmaceutically effective dosages as determined using standard techniques. Preferably the dosing regimen will be accompanied by appropriate diagnostic or monitoring techniques that allow it to be modified.

Yet other embodiments of the invention comprise administering the disclosed CLDN CAR treatments to subjects that are asymptomatic but at risk of developing a proliferative disorder. That is, the CLDN CAR treatments of the instant invention may be used in a truly preventative sense and given to patients that have been examined or tested and have one or more noted risk factors (e.g., genomic indications, family history, in vivo or in vitro test results, etc.) but have not developed neoplasia. In such cases those skilled in the art would be able to determine an effective dosing regimen through empirical observation or through accepted clinical practices.

### IX. Combination Therapies

As previously discussed It will be appreciated that the CLDN CAR treatments described herein may be used in combination with other clinical oncology treatments. In general the treatments of the instant invention may be used with a therapeutic moiety or a drug such as an anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Combination therapies may be useful in preventing or treating cancer and in preventing metastasis or recurrence of cancer. “Combination therapy”, as used herein, means the administration of a combination comprising at least one CLDN CAR treatment and at least one therapeutic moiety (e.g., anti-cancer agent) wherein the combination preferably has therapeutic synergy or improves the measurable therapeutic effects in the treatment of cancer over (i) the CLDN CAR treatment used alone, or (ii) the therapeutic moiety used alone, or (iii) the use of the therapeutic moiety in combination with another therapeutic moiety without the addition of CLDN CAR treatment. The term “therapeutic synergy”, as used herein, means the combination of an CLDN CAR treatment and one or more therapeutic moiety(ies) having a therapeutic effect greater than the additive effect of the combination of the CLDN CAR treatment and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of CLDN CAR treatments described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable.)

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or CLDN CAR treatment, or the sum of the therapeutic effects elicited by the CLDN CAR treatment or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or CLDN CAR treatment or the sum of the therapeutic effects elicited by the CLDN CAR treatment or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the CLDN CAR treatment and therapeutic moiety(ies) may be administered to the subject simultaneously, either in a single composition, or as two or more distinct compositions using the same or different administration routes. Alternatively, treatment with the CLDN CAR treatment may precede or follow the therapeutic moiety treatment by, e.g., intervals ranging from minutes to weeks. In one embodiment, both the therapeutic moiety and the CAR are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the CAR and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The CAR and therapeutic moiety(ies) may be administered on alternate days or weeks; or a sequence of CLDN CAR treatments may be given, followed by one or more treatments with the additional therapeutic moiety. In one embodiment an CLDN CAR is administered in combination with one or more therapeutic moiety(ies) for short treatment cycles. In other embodiments the combination treatment is administered for long treatment cycles. The combination therapy can be administered via any route.

In some embodiments the CLDN CAR treatment (i.e. administration of CLDN sensitized lymphocytes) may be used in combination with various first line cancer treatments. In one embodiment the combination therapy comprises the use of a CLDN CAR treatment and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies).

PD-1, together with its ligand PD-L1, is another negative regulator of the antitumor T lymphocyte response. In one embodiment the combination therapy may comprise CLDN CAR treatment together with an anti-PD-L1 antibody (e.g. lambrolizumab, nivolumab) and optionally one or more other therapeutic moiety(ies). In another embodiment the combination therapy may comprise CLDN CAR treatment together with an anti-PD-L1 antibody (e.g. MPDL3280A, MED14736) and optionally one or more other therapeutic moiety(ies). In yet another embodiment, the combination therapy may comprise a CLDN CAR treatment together with an anti PD-1 antibody (e.g., pembrolizumab) administered to patients who continue progress following treatments with other anti-PD-1 and/or targeted BRAF combination therapies (e.g., ipilimumab and vemurafenib or dabrafinib).

In another embodiment the combination therapy comprises the use of an CLDN CAR treatment and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

In one embodiment, for example, in the treatment of BR-ERPR, BR-ER or BR-PR cancer, the combination therapy comprises the use of a CLDN CAR treatment and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of an CLDN CAR treatment and trastuzumab or ado-trastuzumab emtansine and optionally one or more other therapeutic moiety(ies) (e.g. pertuzumab and/or docetaxel).

In some embodiments, for example, in the treatment of metastatic breast cancer, the combination therapy comprises the use of an CLDN CAR treatment and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of an CLDN CAR treatment and megestrol and optionally an additional therapeutic moiety(ies).

In further embodiments, for example, in the treatment of BR-TNBC, the combination therapy comprises the use of an CLDN CAR treatment and a poly ADP ribose polymerase (PARP) inhibitor (e.g. BMN-673, olaparib, rucaparib and veliparib) and optionally an additional therapeutic moiety(ies).

In another embodiment, for example, in the treatment of breast cancer, the combination therapy comprises the use of an CLDN CAR treatment and cyclophosphamide and optionally an additional therapeutic moiety(ies) (e.g. doxorubicin, a taxane, epirubicin, 5-FU and/or methotrexate.

In another embodiment combination therapy for the treatment of EGFR-positive NSCLC comprises the use of an CLDN CAR treatment and afatinib and optionally one or more other therapeutic moiety(ies) (e.g. erlotinib and/or bevacizumab).

In another embodiment combination therapy for the treatment of EGFR-positive NSCLC comprises the use of an CLDN CAR treatment and erlotinib and optionally one or more other therapeutic moiety(ies) (e.g. bevacizumab).

In another embodiment combination therapy for the treatment of ALK-positive NSCLC comprises the use of an CLDN CAR treatment and ceritinib and optionally one or more other therapeutic moiety(ies).

In another embodiment combination therapy for the treatment of ALK-positive NSCLC comprises the use of an CLDN CAR treatment and crizotinib and optionally one or more other therapeutic moiety(ies).

In another embodiment the combination therapy comprises the use of an CLDN CAR treatment and bevacizumab and optionally one or more other therapeutic moiety(ies) (e.g. a taxane such as, for example, docetaxel or paclitaxel; and/or a platinum analog).

In another embodiment the combination therapy comprises the use of an CLDN CAR treatment and bevacizumab and optionally one or more other therapeutic moiety(ies) (e.g. gemcitabine and/or a platinum analog).

In one embodiment the combination therapy comprises the use of an CLDN CAR treatment and a platinum-based drug (e.g. carboplatin or cisplatin) analog and optionally one or more other therapeutic moiety(ies) (e.g. a taxane such as, for example, docetaxel and paclitaxel).

In one embodiment the combination therapy comprises the use of an CLDN CAR treatment and platinum-based drug (e.g. carboplatin or cisplatin) analog and optionally one or more other therapeutic moiety(ies) (e.g. a taxane such, for example, docetaxel and paclitaxel and/or gemcitabine and/or doxorubicin).

In a particular embodiment the combination therapy for the treatment of platinum-resistant tumors comprises the use of a CLDN CAR treatment and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In another embodiment the combination therapy comprises the use of a CLDN CAR treatment and a PARP inhibitor and optionally one or more other therapeutic moiety(ies).

In another embodiment the combination therapy comprises the use of a CLDN CAR treatment and bevacizumab and optionally cyclophosphamide.

The combination therapy may comprise a CLDN CAR treatment and a chemotherapeutic moiety that is effective on a tumor comprising a mutated or aberrantly expressed gene or protein (e.g. BRCA1).

More generally the CLDN CAR treatments of the instant invention may be used in combination with a number of anti-cancer agents. The term “anti-cancer agent” or “chemotherapeutic agent” as used herein is one subset of “therapeutic moieties”, which in turn is a subset of the agents described as “pharmaceutically active moieties”. More particularly “anti-cancer agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. It will be appreciated that in selected embodiments as discussed above, such anti-cancer agents may comprise antibody drug conjugates and may be associated with antibodies prior to administration.

The term “cytotoxic agent,” which can also be an anti-cancer agent means a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca americana proteins (PAPI, PAPII, and PAP-S), Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

An anti-cancer agent can include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. Such agents are often administered, and are often most effective, in combination, e.g., in the formulation CHOP.

Examples of anti-cancer agents that may be used in combination with CLDN CAR treatment of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifene citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

“Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the CLDN CAR treatments of the instant invention may be used in combination with any one of a number of antibodies (or immunotherapeutic agents) presently in clinical trials or commercially available. To this end the disclosed CLDN sensitized lymphocytes may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lambrolizumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MDX-1105 and MED14736 and combinations thereof.

Other particularly preferred embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

The present invention also provides for the combination of the CLDN CAR treatments with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed CLDN CAR treatments may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### X. Diagnostics

The invention provides in vitro and in vivo methods for detecting, diagnosing or monitoring the efficiency of any lymphocyte transduction or the effect of any CLDN sensitized lymphocytes on tumor cells including tumorigenic cells. Such methods include identifying an individual having cancer (e.g., a CLDN positive tumor) for treatment or monitoring progression of a cancer, comprising interrogating the patient or a sample obtained from a patient (either in vivo or in vitro) with an antibody as described herein before during or after treatment with CLDN sensitized lymphocytes and detecting presence or absence, or level of association, of the antibody to bound or free target molecules in the sample. In some embodiments the CLDN antibody will comprise a detectable label or reporter molecule as described herein. In yet other embodiments (e.g., In situ hybridization or ISH) a nucleic acid probe that reacts with a genomic CLDN determinant will be used in the detection, diagnosis or monitoring of the proliferative disorder.

More generally the presence and/or levels of CLDN determinants may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with the compositions as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.) or In situ hybridization (ISH) or variants thereof (e.g., chromogenic in situ hybridization (CISH) or fluorescence in situ hybridization (DNA-FISH or RNA-FISH)).

In this regard certain aspects of the instant invention comprise the use of labeled CLDN for immunohistochemistry (IHC). More particularly CLDN IHC may be used as a diagnostic tool to aid in the diagnosis of various proliferative disorders and to monitor the potential response to treatments including CLDN antibody therapy. As discussed herein and shown in the Examples below compatible diagnostic assays may be performed on tissues that have been chemically fixed (including but not limited to: formaldehyde, gluteraldehyde, osmium tetroxide, potassium dichromate, acetic acid, alcohols, zinc salts, mercuric chloride, chromium tetroxide and picric acid) and embedded (including but not limited to: glycol methacrylate, paraffin and resins) or preserved via freezing. Such assays can be used to guide treatment decisions and determine dosing regimens and timing.

Other particularly compatible aspects of the invention involve the use of in situ hybridization to detect or monitor CLDN determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic CLDN determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected CLDN determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In a particularly preferred embodiment the antibodies disclosed herein may be used to detect and quantify levels of a particular determinant (e.g., CLDN) in a patient sample (e.g., plasma or blood) which may, in turn, be used to detect, diagnose or monitor proliferative disorders both before and after treatment with the CLDN sensitized lymphocytes. In related embodiments the antibodies disclosed herein may be used to detect, monitor and/or quantify circulating tumor cells either in vivo or in vitro (WO 2012/0128801) in combination with the disclosed treatments by CLDN sensitized lymphocytes. In still other embodiments the circulating tumor cells may comprise tumorigenic cells.

In certain embodiments of the invention, the tumorigenic cells in a subject or a sample from a subject may be assessed or characterized using the disclosed antibodies prior to CLDN CAR therapy or regimen to establish a baseline. In other examples, the tumorigenic cells can be assessed from a sample that is derived from a subject that was treated.

Xl. Articles of Manufacture

The invention further includes pharmaceutical packs and kits comprising one or more containers, wherein a container can comprise one or more transformation doses of a CLDN CAR plasmid or vector of the invention. In certain embodiments, the pack or kit contains a vector preparation (e.g., lentiviral or retroviral) comprising a nucleic acid encoding a CLDN CAR, with or without one or more additional reagents and optionally a means of effecting transduction. Preferably the kit will further include the means to monitor and characterize the preparation of the CLDN sensitive lymphocytes prior to administration.

In selected embodiments kits compatible with the invention would allow a user to produce the CLDN sensitive lymphocytes, monitor transduction rates and characterize the resulting CLDN sensitive lymphocyte population to ensure quality prior to administration. Accordingly, a kit of the invention will generally contain a pharmaceutically acceptable formulation of the CAR nucleic acid (or vector) and, optionally, one or more reagents in the same or different containers. In preferred embodiments the CLDN CAR vectors will comprise viral vectors (e.g., lentiviral or retroviral) that allow for transduction of selected host cells to provide the disclosed sensitized lymphocytes. In certain embodiments the selected host cell will be autologous (i.e. derived from the patient to be treated) while in other embodiments the selected host cells will be allogeneic. Some aspects of the invention are directed to kits including allogeneic cells along with the CLDN CAR vector. Yet other embodiments comprise kits or containers incorporating a pharmaceutical composition comprising allogeneic CLDN sensitized lymphocytes. In such kits the container may comprise an infusion bag that would allow the CLDN sensitized lymphocytes to be directly administered to the patient.

The kits may also contain other pharmaceutically acceptable formulations or devices, either for diagnosis or combination therapy. Examples of diagnostic devices or instruments include those that can be used to detect, monitor, quantify or profile cells or markers associated with the CLDN sensitive lymphocytes, transformation efficiency or the proliferative disorder to be treated. In particularly preferred embodiments the devices may be used to detect, monitor and/or quantify circulating tumor cells either in vivo or in vitro. In still other preferred embodiments the circulating tumor cells may comprise tumorigenic cells.

When selected components of the kit are provided in one or more liquid solutions, the liquid solution can be non-aqueous though an aqueous solution is preferred, with a sterile aqueous solution being particularly preferred. The formulations of the kit (e.g., a viral vector) can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the CAR plasmid or vectors of the invention in combination with additional reagents, the solution may be pre-mixed, either in a molar equivalent combination, or with one component in excess of the other. Alternatively, the plasmids of the invention and any optional co-reagents can be maintained separately within distinct containers prior to transformation of the lymphocytes. In other preferred embodiments container(s) of the kit may comprise liquid formulations of allogeneic CLDN sensitized lymphocytes.

The kit can comprise one or multiple containers and a label or package insert in, on or associated with the container(s), indicating that the enclosed composition is used for preparing cells for treating the disease condition of choice. Suitable containers include, for example, bottles, vials, syringes, etc. The containers can be formed from a variety of materials such as glass or plastic. The container(s) can comprise a sterile access port, for example, the container may be an intravenous solution bag or a vial having a stopper that can be pierced by a hypodermic injection needle.

In some embodiments the kit can contain a means by which to administer the sensitized lymphocytes and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### XIII. Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well-known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XIV. References

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described method.

### XV. Sequences

Appended to the instant application are figures and a sequence listing comprising a number of nucleic acid and amino acid sequences. The following Table 2 provides a summary of the included sequences.

TABLE 2

| SEQ | |
|---|---|
| ID | |
| NO | Description |
| 1 | Kappa light chain (LC) constant region protein |
| 2 | IgGl heavy chain (HC) constant region protein |
| 3 | scFv Linker protein |
| 4 | SC27.108 scFv nucleic acid |
| 5 | SC27.108 scFv amino acid |
| 6 | SC27.204v2 scFv nucleic acid |
| 7 | SC27.204v2 scFv amino acid |
| 8 | SCT1-h27.108 nucleic acid |
| 9 | SCT1-h27.108 amino acid |
| 10 | SCT1-h27.204v2 nucleic acid |
| 11 | SCT1-h27.204v2 amino acid |
| 12-19 | Reserved |
| 20 | SC27.1 VL DNA |
| 21 | SC27.1 VL protein |
| 22 | SC27.1 VH DNA |
| 23 | SC27.1 VH protein |
| 24-59 | Additional mouse clones as in SEQ ID NOs: 20-23 |
| 60 | hSC27.1 VL DNA |
| 61 | hSC27.1 VL protein |
| 62 | hSC27.1 VH DNA |
| 63 | hSC27.1 VH protein |
| 64-75 | Additional humanized clones as in SEQ ID NOs: 60-63 |
| 76-77 | hSC27.108v1 VL DNA and protein |
| 78-79 | hSC27.22-VH1-8 VH DNA and protein |
| 80-81 | hSC27.22-VH1-46 VH DNA and protein |
| 82-83 | hSC27.22-VH1-69 VH DNA and protein |
| 84-85 | hSC27.204v1 DNA and protein |
| 86-87 | hSC27.204v2 DNA and protein |
| 88-89 | hSC27.204v3 DNA and protein |
| 90-91 | hSC27.204v4 DNA and protein |
| 92-93 | hSC27.204v5 DNA and protein |
| 94-95 | hSC27.204v6 DNA and protein |
| 96-97 | hSC27.204v7 DNA and protein |
| 98-99 | hSC27.204v8 DNA and protein |
| 100-101 | hSC27.204v9 DNA and protein |
| 102-103 | hSC27.204v10 DNA and protein |
| 104-105 | hSC27.204v11 DNA and protein |
| 106-107 | hSC27.204v12 DNA and protein |
| 108-109 | hSC27.204v13 DNA and protein |
| 110-111 | hSC27.204v14 DNA and protein |
| 112-113 | hSC27.204v15 DNA and protein |
| 114-115 | hSC27.1 full length LC and HC protein |
| 116-117 | hSC27.22 full length LC and HC protein |
| 118-119 | hSC27.108 full length LC and HC protein |
| 120-121 | hSC27.204 full length LC and HC protein |
| 122 | hSC27.22ss1 full length HC protein |
| 123 | hSC27.22-VH1-8 full length HC protein |
| 124 | hSC27.22-VH1-46 full length HC protein |
| 125 | hSC27.22-VH1-69 full length HC protein |
| 126 | hSC27.22 IgG2 full length HC protein |
| 127 | hSC27.22 IgG4 R409K full length HC protein |
| 128 | hSC27.22 IgG4 S228P full length HC protein |
| 129 | hSC27.22 IgG4 S228P K370E R409K full length HC protein |
| 130 | hSC27.22 IgG4 K370E full length HC protein |
| 131 | hSC27.22 IgG4 S228P K370E full length HC protein |
| 132 | hSC27.22 IgG4 C127S S228P full length HC protein |
| 133 | hSC27.22 IgG4 C127S K370E full length HC protein |
| 134 | hSC27.22 IgG4 C127S S228P K370E full length HC protein |
| 135 | hSC27.108v1 full length LC protein |
| 136 | hSC27.204v1 full length HC protein |
| 137 | hSC27.204v2 full length HC protein |
| 138 | hSC27.204v3 full length HC protein |
| 139 | hSC27.204v4 full length HC protein |
| 140 | hSC27.204v5 full length HC protein |
| 141 | hSC27.204v6 full length HC protein |
| 142 | hSC27.204v7 full length HC protein |
| 143 | hSC27.204v8 full length HC protein |
| 144 | hSC27.204v9 full length HC protein |
| 145 | hSC27.204v10 full length HC protein |
| 146 | hSC27.204v11 full length HC protein |
| 147 | hSC27.204v12 full length HC protein |
| 148 | hSC27.204v13 full length HC protein |
| 149 | hSC27.204v14 full length HC protein |
| 150 | hSC27.204v15 full length HC protein |
| 151-156 | hSC27.1 CDRL1; CDRL2; CDRL3, CDRH1; CDRH2; CDRH3 |
| 157-162 | hSC27.22 CDRL1; CDRL2; CDRL3, CDRH1; CDRH2; CDRH3 |
| 163-168 | hSC27.108 CDRL1; CDRL2; CDRL3, CDRH1; CDRH2; |
| | CDRH3 |
| 169-174 | hSC27.204 CDRL1; CDRL2; CDRL3, CDRH1; CDRH2; |
| | CDRH3 |
| 175 | CDRH2 of hSC27.204v1; hSC27.204v5 and hSC27.405v13 |
| 176 | CDRH2 of hSC27.204v2; hSC27.204v6 and hSC27.405v14 |
| 177 | CDRH2 of hSC27.204v3; hSC27.204v7 and hSC27.405v15 |
| 178 | Codon optimized hSC27.22ss1 full length HC DNA |

As discussed in Example 4 below, Table 2 above may further be used to designate SEQ ID NOS for exemplary Kabat CDRs delineated in FIGS. 3A and 3B. More particularly FIGS. 3A and 3B denote the three Kabat CDRs of each heavy (CDRH) and light (CDRL) chain variable region sequence and Table 2 above provides for assignment of a SEQ ID designation that may be applied to each CDRL1, CDRL2 and CDRL3 of the light chain and each CDRH1, CDRH2 and CDRH3 of the heavy chain. Using this methodology each unique CDR set forth in FIGS. 3A and 3B may be assigned a sequential SEQ ID NO and can be used to provide the derived antibodies of the instant invention.

### XVI. Tumor Listing

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 3 as follows:

TABLE 3

| | Abbre- | | Abbre- |
|---|---|---|---|
| Tumor Type | viation | Tumor subtype | viation |
| Breast | BR | | |
| | | estrogen receptor positive | BR-ERPR |
| | | and/or progesterone receptor | |
| | | positive | |
| | | ERBB2/Neu positive | BR- ERBB2/Neu |
| | | HER2 positive | BR-HER2 |
| | | triple-negative | TNBC |
| | | claudin subtype of triple- | TNBC-CLDN |
| | | negative | |
| colorectal | CR | | |
| endometrial | EN | | |
| gastric | GA | | |
| | | diffuse adenocarcinoma | GA-Ad-Dif/Muc |
| | | intestinal adenocarcinoma | GA-Ad-Int |
| | | stromal tumors | GA-GIST |
| glioblastoma | GB | | |
| head and | HN | | |
| neck | | | |
| kidney | KDY | | |
| | | clear renal cell carcinoma | KDY-CC |
| | | papillary renal cell carcinoma | KDY-PAP |
| | | transitional cell or urothelial | KDY-URO |
| | | carcinoma | |
| | | unknown | KDY-UNK |
| liver | LIV | | |
| | | hepatocellular carcinoma | LIV-HCC |
| | | cholangiocarcinoma | LIV-CHOL |
| lymphoma | LN | | |
| lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| melanoma | MEL | | |
| ovarian | OV | | |
| | | clear cell | OV-CC |
| | | endometroid | OV-END |
| | | mixed subtype | OV-MIX |
| | | malignant mixed mesodermal | OV-MMMT |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| pancreatic | PA | | |
| | | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine type | PA-PACe |
| | | ductal adenocarcinoma | PA-PDAC |
| | | ampullary adenocarcinoma | PA-AAC |
| prostate | PR | | |
| skin | SK | | |
| | | melanoma | MEL |
| | | squamous cell carcinomas | SK-SCC |

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Identification of CLDN4, CLDN6 and CLDN9 Expression on Tumors

To characterize the cellular heterogeneity of solid tumors as they exist in cancer patients, aid in the identification of CSCs using particular phenotypic markers and identify clinically relevant therapeutic targets, a large PDX tumor bank was developed and maintained using art recognized techniques. The PDX tumor bank, comprising a large number of discrete tumor cell lines, was propagated in immunocompromised mice through multiple passages of heterogeneous tumor cells originally obtained from numerous cancer patients afflicted by a variety of solid tumor malignancies. The continued availability of a large number of discrete early passage PDX tumor cell lines having well defined lineages greatly facilitates the identification and isolation of CSCs as the PDX tumors allow for the reproducible and repeated characterization of CSCs. The use of minimally passaged PDX tumor cell lines simplifies in vivo experimentation and provides readily verifiable results. Moreover, early passage PDX tumors respond to therapeutic agents such as irinotecan (i.e. Camptosar®), which provides clinically relevant insight into underlying mechanisms driving tumor growth, resistance to current therapies and tumor recurrence.

To generate RNA from the PDX tumor cell lines, tumors were resected from mice after they reached 800-2,000 mm³ and the tumors were dissociated into single cell suspensions using art-recognized enzymatic digestion techniques (see, for example, U.S.P.N. 2007/0292414). Select dissociated PDX tumor cell preparations were depleted of mouse cells and sorted based on their expression of CD46^{hi} and/or CD324, markers of CSC subpopulations (see U.S.P.N 2013/0260385 for the definition of CD46^{hi}). Cells that expressed human EpCAM, CD46^{hi} and/or CD324 (i.e. CSC) or EpCAM but not CD46^{hi} and/or CD324 (i.e. NTG cells), were isolated by FACS using a BD FACSAria cell sorter and lysed in RLTplus RNA lysis buffer (Qiagen) per the manufacturer's instructions. The lysates were then stored at −80° C. and thawed for RNA extraction. Upon thawing, total RNA was extracted using an RNeasy isolation kit (Qiagen, GmbH) following the vendor's instructions and then quantified using a Nanodrop spectrophotometer (Thermo Scientific) and/or a Bioanalyzer 2100 (Agilent Technologies), again using the manufacturer's protocols and recommended instrument settings. The resulting total RNA preparations were assessed by genetic sequencing and gene expression analyses.

Whole transcriptome sequencing of qualified, high quality RNA was performed using an Applied Biosystems (ABI) Sequencing by Oligo Ligation/Detection (SOLiD) 4.5 or SOLiD 5500xl next generation sequencing system (Life Technologies). cDNA was generated from 1 ng total RNA samples using either a modified whole transcriptome protocol from ABI designed for low input total RNA or the Ovation RNA-Seq System V2™ (NuGEN Technologies). The resulting cDNA library was fragmented and barcode adapters were added to allow pooling of fragment libraries from different samples during sequencing runs. Data generated by the SOLiD platform mapped to 34,609 genes as annotated by RefSeq version 47 using NCBI version hg19.2 of the published human genome and provided verifiable measurements of RNA levels in most samples. Sequencing data from the SOLiD platform is nominally represented as a transcript expression value using the metrics RPM (reads per million) or RPKM (read per kilobase per million) mapped to exon regions of genes, enabling basic gene expression analysis to be normalized and enumerated as RPM_Transcript or RPKM_Transcript.

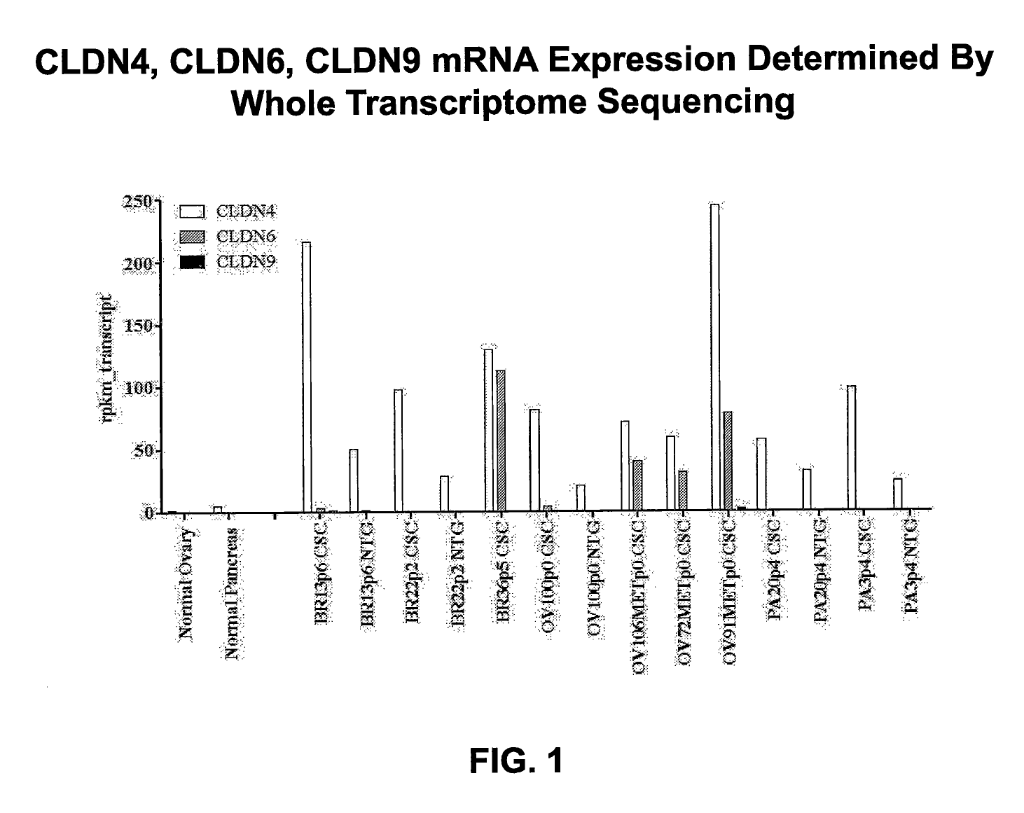

The results of whole transcriptome sequencing using SOLiD showed elevated expression of CLDN4 mRNA in sorted CSC compared to NTG in the following PDX cell lines: BR13, BR22, OV100, PA20 and PA3, as well as high expression in additional CSC populations including BR36, OV106MET, OV72MET, and OV91MET (FIG. 1). CLDN6 mRNA was elevated in sorted CSC populations including BR36, OV106MET, OV72MET, and OV91MET (FIG. 1). Unlike the case for CLDN4 or CLDN6, the related family member, CLDN9, was observed to have low expression in all sorted tumor populations. In contrast to the tumor samples, normal ovary and pancreas tissues showed no or very low mRNA expression of all three family members, CLDN4, CLDN6 and CLDN9.

The identification of elevated CLDN4 and CLDN6 mRNA expression in different types of human tumor indicated these antigens merited further evaluation as a potential diagnostic and/or immunotherapeutic targets.

### Example 2

### Cloning and Expression of Recombinant CLDN Proteins

In order to deduce the relationship between claudin protein sequences, the AlignX program of the Vector NTI software package was used to align 30 claudin protein sequences from 23 human CLDN genes. The results of this alignment are depicted as a dendrogram in FIG. 2A. A review of the figure shows that CLDN6 and CLDN9 are very closely related in sequence, appearing adjacent to one another on the same branch of the dendrogram. FIG. 2A also shows that CLDN4 is the next most closely related family member to CLDN6. A more detailed review of the data shows that the human CLDN6 protein is very closely related to the human CLDN9 protein sequence in the extracellular domains (ECD), with >98% identity in ECD1 and >91% identity in ECD2 (FIG. 2B). Human CLDN4 was also found to be closely related to human CLDN6 in the ECD sequences, with >84% identity in ECD1 and >78% identity in ECD2 (FIG. 2B). Based upon these protein sequence relationships, it was hypothesized that immunization with a human CLDN6 antigen would yield antibodies recognizing human CLDN6 that will also be cross-reactive with human CLDN9, and perhaps also cross-reactive with human CLDN4.

In order to determine which species orthologs of CLDN6, CLDN9 and CLDN4 would be required for screening these multireactive claudin antibodies, ECD sequences of CLDN4, CLDN6 and CLDN9 were analyzed from each of the following species: human, cynomolgus monkey, mouse and rat. The analysis was performed using AlignX and NCBI database protein sequences when available (NP accession numbers of human, mouse and rat proteins are indicated in FIG. 2C). Alternatively, protein sequences were deduced from translation of the cynomolgus monkey CLDN genes assembled by BLAST of human CLDN open reading frame sequences versus cynomolgus monkey whole genome shotgun sequencing contigs. Inspection of these alignments reveals that: (1) deduced cynomolgus monkey protein ECD sequences for CLDN4, CLDN6, and CLDN9 proteins are 100% identical to the respective human ECD sequences; (2) mouse and rat CLDN9 ECD sequences are 100% identical to the human ortholog sequence; (3) and mouse and rat CLDN4 and CLDN6 ECD sequences differ from one another and from the respective human orthologs. Therefore, generation of a set of seven constructs comprising human CLDN4, human CLDN6, human CLDN9, mouse CLDN4, mouse CLDN6, rat CLDN4 and rat CLDN6 should enable determination of cross-reactivity for any binding domain with all possible 12 orthologs.

DNA Fragments Encoding Human CLDN6, CLDN4, and CLDN9 Proteins.

To generate molecular and cellular materials useful in the present invention pertaining to the human CLDN6 (hCLDN6) protein, a codon-optimized DNA fragment encoding a protein identical to NCBI protein accession NP_067018 was synthesized (IDT). This DNA clone was used for all subsequent engineering of constructs expressing the mature hCLDN6 protein or fragments thereof. Similarly, codon-optimized DNA fragments encoding proteins identical to NCBI protein accession NP_001296 for human CLDN4 (hCLDN4), or NCBI protein accession NP_066192 for human CLDN9 (hCLDN9) were purchased and used for all subsequent engineering of constructs expressing the hCLDN4 or hCLDN9 proteins or fragments thereof.

DNA Fragments Encoding Mouse CLDN6 and CLDN4 Proteins.

To generate molecular and cellular materials useful in the present invention pertaining to the mouse CLDN6 (mCLDN6) protein, a codon-optimized DNA fragment encoding a protein identical to NCBI protein accession NP_061247 was synthesized (IDT). This DNA clone was used for all subsequent engineering of constructs expressing the mature mCLDN6 protein or fragments thereof. Similarly, a codon-optimized DNA fragment encoding a protein identical to NCBI protein accession NP_034033 for mouse CLDN4 (mCLDN4) was purchased and used for all subsequent engineering of constructs expressing the mature mCLDN4 protein or fragments thereof.

DNA Fragments Encoding Rat CLDN6 and CLDN4 Proteins.

To generate molecular and cellular materials useful in the present invention pertaining to the rat CLDN6 (rCLDN6) protein, a codon-optimized DNA fragment encoding a protein identical to NCBI protein accession NP_001095834 was synthesized (IDT). This DNA clone was used for all subsequent engineering of constructs expressing the mature rCLDN6 protein or fragments thereof. Similarly, a codon-optimized DNA fragment encoding a protein identical to NCBI protein accession NP_001012022 for rat CLDN4 (rCLDN4) was purchased and used for all subsequent engineering of constructs expressing the mature rCLDN4 protein or fragments thereof.

Cell Line Engineering

Engineered cell lines overexpressing the various CLDN proteins listed above were constructed using lentiviral vectors to transduce HEK-293T or 3T3 cell lines using art recognized techniques. First, PCR was used to amplify the DNA fragments encoding the protein of interest (e.g., hCLDN6, mCLDN6, rCLDN6, hCLDN9, hCLDN4, mCLDN4, or rCLDN4) using the commercially synthesized DNA fragments described above as templates. Then, the individual PCR products were subcloned into the multiple cloning site (MCS) of the lentiviral expression vector, pCDH-EF1-MCS-T2A-GFP (System Biosciences), to generate a suite of lentiviral vectors. The T2A sequence in resultant pCDH-EF1-CLDN-T2A-GFP vectors promotes ribosomal skipping of a peptide bond condensation, resulting in expression of two independent proteins: high level expression of the specific CLDN protein encoded upstream of the T2A peptide, with co-expression of the GFP marker protein encoded downstream of the T2A peptide. This suite of lentiviral vectors was used to create separate stable HEK-293T or 3T3 cell lines overexpressing individual CLDN proteins using standard lentiviral transduction techniques well known to those skilled in the art. CLDN-positive cells were selected with FACS using high-expressing HEK-293T subclones, which were also strongly positive for GFP.

### Example 3

### Generation of Anti-CLDN Antibodies

Because CLDN6 is most homologous to CLDN4 and CLDN9 (see FIG. 2A and analysis as described in Example 2, above), CLDN6 was used as the immunogen with which to generate multireactive anti-CLDN antibodies. Mice were inoculated with HEK-293T cells or 3T3 cells overexpressing hCLDN6 (generated as described in Example 2) in order to produce antibody-generating hybridomas. Six mice (two each of the following strains: Balb/c, CD-1, FVB) were inoculated with 1 million hCLDN6-HEK-293T cells emulsified with an equal volume of TiterMax® adjuvant. A second, separate inoculation of six mice (two each of the following strains: Balb/c, CD-1, FVB) was performed using 3T3 cells overexpressing CLDN6. Following the initial inoculation the mice were injected twice weekly for 4 weeks with cells overexpressing CLDN6 emulsified with an equal volume of alum adjuvant.

Mice were sacrificed and draining lymph nodes (popliteal, inguinal, and medial iliac) were dissected and used as a source for antibody producing cells. A single cell suspension of B cells (305×10⁶ cells) were fused with non-secreting P3×63Ag8.653 myeloma cells (ATCC #CRL-1580) at a ratio of 1:1 by electro cell fusion using a model BTX Hybrimmune System (BTX Harvard Apparatus). Cells were resuspended in hybridoma selection medium: DMEM medium (Cellgro) supplemented with azaserine (Sigma), 15% fetal clone I serum (Hyclone), 10% BM condimed (Roche Applied Sciences), 1 mM sodium pyruvate, 4 mM L-glutamine, 100 IU penicillin-streptomycin, 50 μM 2-mercaptoethanol, and 100 μM hypoxanthine, and cultured in three T225 flasks in 90 mL selection medium per flask. The flasks were placed in a humidified 37° C. incubator containing 5% CO₂ and 95% air for 6 days. The library was frozen down in 6 vials of CryoStor CS10 buffer (BioLife Solutions), with approximately 15×10⁶ viable cells per vial, and stored in liquid nitrogen.

One vial from the library was thawed at 37° C. and the frozen hybridoma cells were added to 90 mL hybridoma selection medium, described above, and placed in a T150 flask. The cells were cultured overnight in a humidified 37° C. incubator with 5% CO₂ and 95% air. The following day hybridoma cells were collected from the flask and plated at one cell per well (using a FACSAria I cell sorter) in 200 μL of supplemented hybridoma selection medium into 48 Falcon 96-well U-bottom plates. The hybridomas were cultured for 10 days and the supernatants were screened for antibodies specific to hCLDN6, hCLDN4 or hCLDN9 proteins using flow cytometry. Flow cytometry was performed as follows: 1×10⁵ per well of HEK-293T cells, stably transduced with lentiviral vectors encoding hCLDN6, hCLDN4 or hCLDN9, were incubated for 30 mins. with 100 μL hybridoma supernatant. Cells were washed with PBS/2% FCS and then incubated with 50 μL per sample DyeLight 649 labeled goat-anti-mouse IgG, Fc fragment specific secondary antibody diluted 1:200 in PBS/2%FCS. After a 15 min. incubation cells were washed twice with PBS/2%FCS and re-suspended in PBS/2%FCS with DAPI (to detect dead cells) and analyzed by flow cytometry for fluorescence exceeding that of cells stained with an isotype control antibody. Selected hybridomas that tested positive for antibodies directed to one or more of the CLDN antigens were set aside for further characterization. Remaining, unused hybridoma library cells were frozen in liquid nitrogen for future library testing and screening.

### Example 4

### Sequencing of Anti-CLDN Antibodies

Anti-CLDN antibodies were generated as described above and then sequenced as follows. Total RNA was purified from selected hybridoma cells using the RNeasy Miniprep Kit (Qiagen) according to the manufacturer's instructions. Between 10⁴ and 10⁵ cells were used per sample. Isolated RNA samples were stored at −80° C. until used. The variable region of the Ig heavy chain of each hybridoma was amplified using two 5′ primer mixes comprising 86 mouse specific leader sequence primers designed to target the complete mouse VH repertoire in combination with a 3′ mouse Cγ primer specific for all mouse Ig isotypes. Similarly, two primer mixes containing 64 5′ VK leader sequences designed to amplify each of the VK mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of four RT-PCR reactions were run for each hybridoma, two for the VK light chain and two for the VH heavy chain. PCR reaction mixtures included 1.5 μL of RNA, 0.4 μL of 100 μM of either heavy chain or kappa light chain primers (custom synthesized by IDT), 5 μL of 5× RT-PCR buffer, 1 μL dNTPs, and 0.6 μL of enzyme mix containing reverse transcriptase and DNA polymerase. The thermal cycler program included the following steps: RT step 50° C. for 60 min., 95° C. for 15 min. followed by 35 cycles of (94.5° C. for 30 seconds, 57° C. for 30 seconds, 72° C. for 1 min.), and a final incubation at 72° C. for 10 min. The extracted PCR products were sequenced using the same specific variable region primers as described above. PCR products were sent to an external sequencing vendor (MCLAB) for PCR purification and sequencing services.

FIG. 3A depicts the contiguous amino acid sequences of several novel mouse light chain variable regions from anti-CLDN antibodies (SEQ ID NOS: 21-57, odd numbers). FIG. 3B depicts the contiguous amino acid sequences of novel mouse heavy chain variable regions from the same anti-CLDN antibodies (SEQ ID NOS: 23-59, odd numbers). Mouse light and heavy chain variable region nucleic acid sequences are provided in FIG. 3C (SEQ ID NOS: 20-58, even numbers). Taken together FIGS. 3A and 3B provide the annotated sequences of 10 mouse anti-CLDN antibodies, termed SC27.1, SC27.22, SC27.103, SC27.104, SC27.105, SC27.106, SC27.108 (identical to SC27.109), SC27.201, SC27.203 and SC27.204. The amino acid sequences are annotated to identify the framework regions (i.e. FR1-FR4) and the complementarity determining regions (i.e. CDRL1-CDRL3 in FIG. 3A or CDRH1-CDRH3 in FIG. 3B) defined as per Kabat. The variable region sequences were analyzed using a proprietary version of the Abysis database to provide the CDR and FR designations. Though the CDRs are numbered according to Kabat those skilled in art will appreciate that the CDR and FR designations can also be defined according to Chothia, McCallum or any other accepted nomenclature system.

The SEQ ID NOS of each particular antibody are sequential odd numbers. Thus the monoclonal anti-CLDN antibody, SC27.1, comprises amino acid SEQ ID NOS: 21 and 23 for the VL and VH, respectively; and SC27.22 comprises SEQ ID NOS: 25 and 27 etc. The corresponding nucleic acid sequence for each antibody amino acid sequence is included in FIG. 3C and has the SEQ ID NO immediately preceding the corresponding amino acid SEQ ID NO. Thus, for example, the SEQ ID NOS of the nucleic acid sequences of the VL and VH of the SC27.1 antibody are SEQ ID NOS: 20 and 22, respectively.

### Example 5

### Generation of Chimeric and Humanized Anti-CLDN Antibodies

Chimeric anti-CLDN antibodies were generated using art-recognized techniques as follows. Total RNA was extracted from the anti-CLDN antibody-producing hybridomas using standard biochemical techniques and the RNA was PCR amplified. Data regarding V, D and J gene segments of the VH and VL chains of the mouse antibodies were obtained from the nucleic acid sequences of the anti-CLDN antibodies of the invention (see FIG. 3C for nucleic acid sequences). Primer sets specific to the framework sequence of the VH and VL chain of the antibodies were designed using the following restriction sites: Agel and Xhol for the VH fragments, and Xmal and Dralll for the VL fragments. PCR products were purified with a Qiaquick PCR purification kit (Qiagen), followed by digestion with restriction enzymes Agel and Xhol for the VH fragments and Xmal and Dralll for the VL fragments. The VH and VL digested PCR products were purified and ligated into IgH or IgK expression vectors, respectively. Ligation reactions were performed in a total volume of 10 μL with 200 U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the VH fragment was cloned into the Agel-Xhol restriction sites of the pEE6.4 expression vector (Lonza) comprising HulgG1 (pEE6.4HulgG1) and the VL fragment was cloned into the Xmal-Dralll restriction sites of the pEE12.4 expression vector (Lonza) comprising a human kappa light constant region (pEE12.4Hu-Kappa).

Chimeric antibodies were expressed by co-transfection of either HEK-293T or CHO-S cells with pEE6.4HulgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 2.5 μg each of pEE6.4HulgG1 and pEE12.4Hu-Kappa vector DNA were added to 10 μL HEK-293T transfection reagent in 1.5 mL Opti-MEM. The mix was incubated for 30 min. at room temperature and added to cells. Supernatants were harvested three to six days after transfection. For CHO-S cells, 2.5 μg each of pEE6.4HulgG1 and pEE12.4Hu-Kappa vector DNA were added to 15 μg PEI transfection reagent in 400 μL Opti-MEM. The mix was incubated for 10 min. at room temperature and added to cells. Supernatants were harvested three to six days after transfection. Culture supernatants containing recombinant chimeric antibodies were cleared from cell debris by centrifugation at 800×g for 10 min. and stored at 4° C. Recombinant chimeric antibodies were purified with Protein A beads

Mouse anti-CLDN antibodies were humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences, and the framework sequences and CDRs of the relevant mouse antibodies. For the purpose of the analysis the assignment of amino acids to each of the CDR domains was done in accordance with Kabat numbering. Once the variable regions were selected, they were generated from synthetic gene segments (Integrated DNA Technologies). Humanized antibodies were cloned and expressed using the molecular methods described above for chimeric antibodies.

The VL and VH amino acid sequences of the humanized antibodies were derived from the VL and VH sequences of the corresponding mouse antibody (e.g. hSC27.1 is derived from mouse SC27.1). There were no framework changes or back mutations made in the light or heavy chain variable regions of the four humanized antibodies generated: hSC27.1,hSC27.22, hSC17.108 and hSC27.204.

To address stability concerns, three variants of hSC27.22 were produced using different VH frameworks in the same VH1 family. The variants were termed hSC27.22-VH1-8; hSC27.22-VH1-46; hSC27.22-VH1-69. In addition, one variant of hSC27.108 was constructed, termed hSC27.108v1, which shares the same heavy chain as hSC27.108 (SEQ ID NO: 119) but differs in light chain compared to hSC27.108. In addition, several variants of hSC27.204 were generated, termed hSC27.204v1 through hSC27.204v15, all of which share the same light chain (SEQ ID NO: 120) but differ in the heavy chain. The heavy chains of hSC27.204 and hSC27.204v4 differ in a single framework region mutation, T28D. hSC27.204v1 through hSC27.204v3 and hSC27.204v5 through hSC27.204v7 incorporate conservative mutations in the CDRs to address stability concerns. Specifically, hSC27.204v1, hSC27.204v2, and hSC27.204v3 contain the modifications N58K, N58Q, and T60N, respectively, on the hSC27.204 heavy chain background. Similarly, hSC27.204v5, hSC27.204v6, and hSC27.204v7 contain the modifications N58K, N58Q, and T60N, respectively on the hSC27.204v4 background. Lastly, variants hSC27.204v8 and hSC27.204v9 do not include a back mutation at position 93 of the heavy chain in order to minimize immunogenicity. Specifically, variants hSC27.204v8, hSC27.204v9, hSC27.204v10, hSC27.204v11, hSC27.204v12, hSC27.204v13, hSC27.204v14, and hSC27.204v15 correspond to variants hSC27.204, hSC27.204v1, hSC27.204v2, hSC27.204v3, hSC27.204v4, hSC27.204v5, hSC27.204 6, and hSC27.204v7, respectively, except that variants 8-15 lack the A93T back mutation.

In addition, 9 variants of the hSC27.22 humanized antibody constant region were constructed. The first variant, hSC27.22ss1 is a site specific variant and is described in more detail in Example 8 below. The other variants were constructed by substituting the IgG isotype with either IgG2 (termed, “hSC27.22 IgG2”) or mutated forms of IgG4 (termed, “hSC27.22 IgG4 R409K”; “hSC27.22 IgG4 S228P”; “hSC27.22 IgG4 S228P K370E R409K”; “hSC27.22 IgG4 K370E”; “hSC27.22 IgG4 S228P K370E”; “hSC27.22 IgG4 C127S S228P”; “hSC27.22 IgG4 C127S K370E”; and “hSC27.22 IgG4 C127S S228P K370E”). Table 4 below shows a summary of the humanized anti CLDN antibodies and their variants, with residue changes numbered according to Kabat et al.

In each case, the binding affinity of the humanized antibody was checked to ensure that it was substantially equivalent to the corresponding mouse antibody. FIG. 3A depicts the contiguous amino acid sequences of the VL of exemplary humanized antibodies and their variants. FIG. 3B depicts the contiguous amino acid sequences of the VH of exemplary humanized antibodies and their variants. The nucleic acid sequences of the light and heavy chain variable regions of the anti-CLDN humanized antibodies are provided in FIG. 3C.

TABLE 4

| | | | | VH FR | VH CDR | | | VK FR | VK CDR |
|---|---|---|---|---|---|---|---|---|---|
| mAb | Isotype | Human VH | hJH | Changes | Changes | Human VK | hJK | Changes | Changes |
| hSC27.1 | IgG1 | IGHV1-3*01 | JH1 | None | None | IGKV1-12*01 | JK2 | None | None |
| hSC27.22 | IgG1 | IGHV1-3*01 | JH6 | None | None | IGKV4-1*01 | JK2 | None | None |
| hSC27.22ss1 | IgG1 | IGHV1-8*01 | JH6 | None | None | IGKV4-1*01 | JK2 | None | None |
| | C220S | | | | | | | | |
| hSC27.22- | IgG1 | IGHV1-8*01 | JH6 | None | None | IGKV4-1*01 | JK2 | None | None |
| VH1-8 | | | | | | | | | |
| hSC27.22 | IgG1 | IGHV1-46*01 | JH6 | None | None | IGKV4-1*01 | JK2 | None | None |
| VH1-46 | | | | | | | | | |
| hSC27.22 | IgG1 | IGHV1-69*01 | JH6 | None | None | IGKV4-1*01 | JK2 | None | None |
| VH1-69 | | | | | | | | | |
| hSC27.22 | IgG2 | IGHV1-3*01 | JH6 | None | None | IGKV4-1*01 | JK2 | None | None |
| IgG2 | | | | | | | | | |
| hSC27.22 | IgG4 | IGHV1-3*01 | JH6 | None | None | IGKV4-1*01 | JK2 | None | None |
| IgG4 R409K | R409K | | | | | | | | |
| hSC27.22 | IgG4 | IGHV1-3*01 | JH6 | None | None | IGKV4-1*01 | JK2 | None | None |
| IgG4 S228P | S228P | | | | | | | | |
| hSC27.22 | IgG4 | IGHV1-3*01 | JH6 | None | None | IGKV4-1*01 | JK2 | None | None |
| IgG4 S228P | S228P | | | | | | | | |
| K370E R409K | R409K | | | | | | | | |
| | K370E | | | | | | | | |
| hSC27.22 | IgG4 | IGHV1-3*01 | JH6 | None | None | IGKV4-1*01 | JK2 | None | None |
| IgG4 K370E | K370E | | | | | | | | |
| hSC27.22 | IgG4 | IGHV1-3*01 | JH6 | None | None | IGKV4-1*01 | JK2 | None | None |
| IgG4 S228P | S228P | | | | | | | | |
| K370E | K370E | | | | | | | | |
| hSC27.22 | IgG4 | IGHV1-3*01 | JH6 | None | None | IGKV4-1*01 | JK2 | None | None |
| IgG4 C127S | C127S | | | | | | | | |
| S228P | S228P | | | | | | | | |
| hSC27.22 | IgG4 | IGHV1-3*01 | JH6 | None | None | IGKV4-1*01 | JK2 | None | None |
| IgG4 C127S | C127S | | | | | | | | |
| K370E | K370E | | | | | | | | |
| hSC27.22 | IgG4 | IGHV1-3*01 | JH6 | None | None | IGKV4-1*01 | JK2 | None | None |
| IgG4 C127S | C127S | | | | | | | | |
| S228P K370E | S228P | | | | | | | | |
| | K370E | | | | | | | | |
| hSC27.108 | IgG1 | IGHV1-18*01 | JH1 | None | None | IGKV3-11*01 | JK4 | None | None |
| hSC27.108 v1 | IgG1 | IGHV1-18*01 | JH1 | None | None | IGKV6-21*01 | JK4 | L47W | None |
| | | | | | | | | K49Y | |
| hSC27.204 | IgG1 | IGHV3-23*01 | JH1 | A93T | None | IGKV1-16*01 | JK4 | None | None |
| | | | | K94G | | | | | |
| hSC27.204 v1 | IgG1 | IGHV3-23*01 | JH1 | A93T | N58K | IGKV1-16*01 | JK4 | None | None |
| | | | | K94G | | | | | |
| hSC27.204v2 | IgG1 | IGHV3-23*01 | JH1 | A93T | N58Q | IGKV1-16*01 | JK4 | None | None |
| | | | | K94G | | | | | |
| hSC27.204v3 | IgG1 | IGHV3-23*01 | JH1 | A93T | T60N | IGKV1-16*01 | JK4 | None | None |
| | | | | K94G | | | | | |
| hSC27.204v4 | IgG1 | IGHV3-23*01 | JH1 | T28D, | None | IGKV1-16*01 | JK4 | None | None |
| | | | | A93T | | | | | |
| | | | | K94G | | | | | |
| hSC27.204v5 | IgG1 | IGHV3-23*01 | JH1 | T28D, | N58K | IGKV1-16*01 | JK4 | None | None |
| | | | | A93T | | | | | |
| | | | | K94G | | | | | |
| hSC27.204v6 | IgG1 | IGHV3-23*01 | JH1 | T28D, | N58Q | IGKV1-16*01 | JK4 | None | None |
| | | | | A93T | | | | | |
| | | | | K94G | | | | | |
| hSC27.204v7 | IgG1 | IGHV3-23*01 | JH1 | T28D, | T60N | IGKV1-16*01 | JK4 | None | None |
| | | | | A93T | | | | | |
| | | | | K94G | | | | | |
| hSC27.204v8 | IgG1 | IGHV3-23*01 | JH1 | K94G | None | IGKV1-16*01 | JK4 | None | None |
| hSC27.204v9 | IgG1 | IGHV3-23*01 | JH1 | K94G | N58K | IGKV1-16*01 | JK4 | None | None |
| hSC27.204v10 | IgG1 | IGHV3-23*01 | JH1 | K94G | N58Q | IGKV1-16*01 | JK4 | None | None |
| hSC27.204v11 | IgG1 | IGHV3-23*01 | JH1 | K94G | T60N | IGKV1-16*01 | JK4 | None | None |
| hSC27.204v12 | IgG1 | IGHV3-23*01 | JH1 | T28D, | None | IGKV1-16*01 | JK4 | None | None |
| | | | | K94G | | | | | |
| hSC27.204v13 | IgG1 | IGHV3-23*01 | JH1 | T28D, | N58K | IGKV1-16*01 | JK4 | None | None |
| | | | | K94G | | | | | |
| hSC27.204v14 | IgG1 | IGHV3-23*01 | JH1 | T28D, | N58Q | IGKV1-16*01 | JK4 | None | None |
| | | | | K94G | | | | | |
| hSC27.204v15 | IgG1 | IGHV3-23*01 | JH1 | T28D, | T60N | IGKV1-16*01 | JK4 | None | None |
| | | | | K94G | | | | | |

It will be appreciated that each of the aforementioned antibodies, or immunoreactive fragments thereof, may be used to provide CLDN CAR binding domains in accordance with the instant disclosure.

### Example 6

### Specificity of Anti-CLDN Antibodies

The mouse antibodies generated as described in Example 3, were characterized to determine whether they cross reacted with CLDN family members and orthologs of CLDN family members.

Flow cytometry analyses were performed as follows: HEK-293T cells were stably transduced with (i) lentiviral vectors encoding hCLDN6, mCLDN6, and rCLDN6; (ii) hCLDN9; or (iii) hCLDN4, mCLDN4 and rCLDN4, made as described in Example 4 above. 1×10⁵ HEK-293T cells stably transduced with the aforementioned expression constructs were incubated at 4° C. for 30 mins. with either hSC27.1 or hSC27.22 antibodies, diluted to 10 μg/ml into a final volume of 50 μl PBS/2%FCS. Following incubation, cells were washed with 200 μL PBS/2%FCS, pelleted by centrifugation, supernatant was discarded, and cell pellets were resuspended in 50 μL per sample DyeLight 649 labeled goat-anti-mouse IgG, Fc fragment specific secondary antibody diluted 1:200 in PBS/2%FCS. After a 15 min. incubation at 4° C. cells were washed and pelleted twice with PBS/2%FCS as previously described and resuspended in 100 μL PBS/2%FCS with 2 μg/mL 4′,6-diamidino-2-phenylindole dihydrochloride (DAPI). Samples were analyzed by flow cytometry and live cells were assessed with DyeLight 649 for fluorescence exceeding that of cells stained with an isotype control antibody.

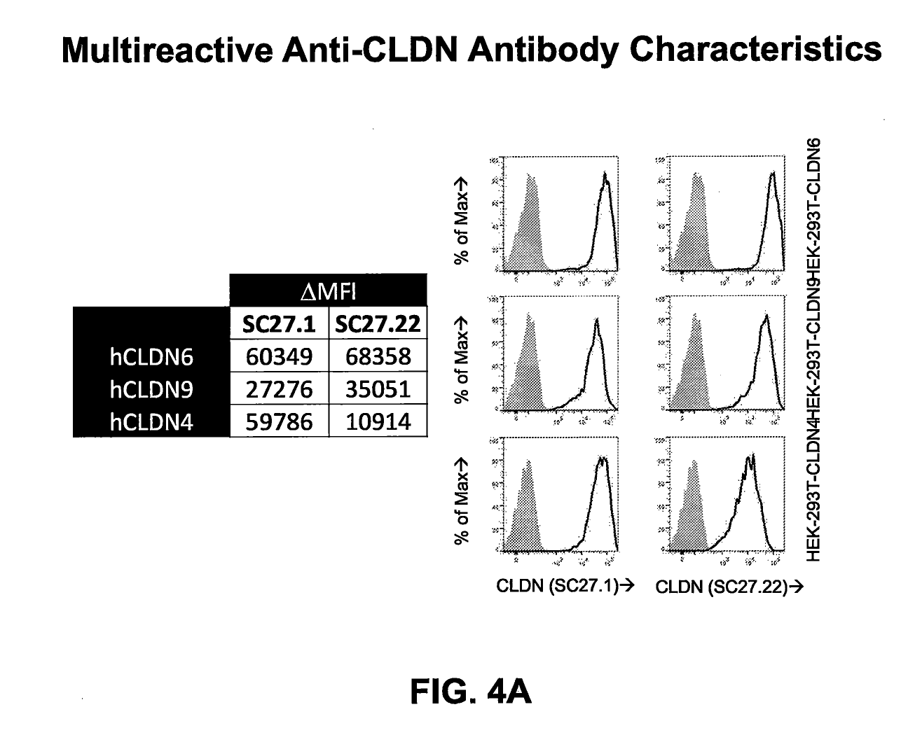

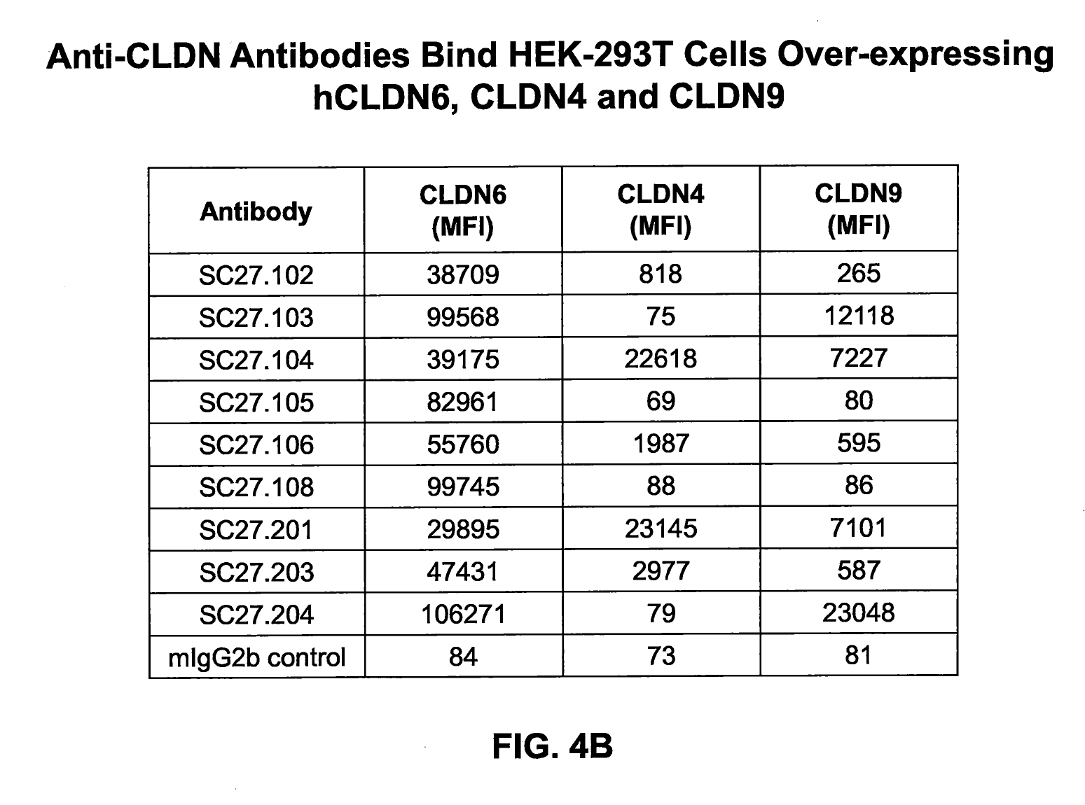

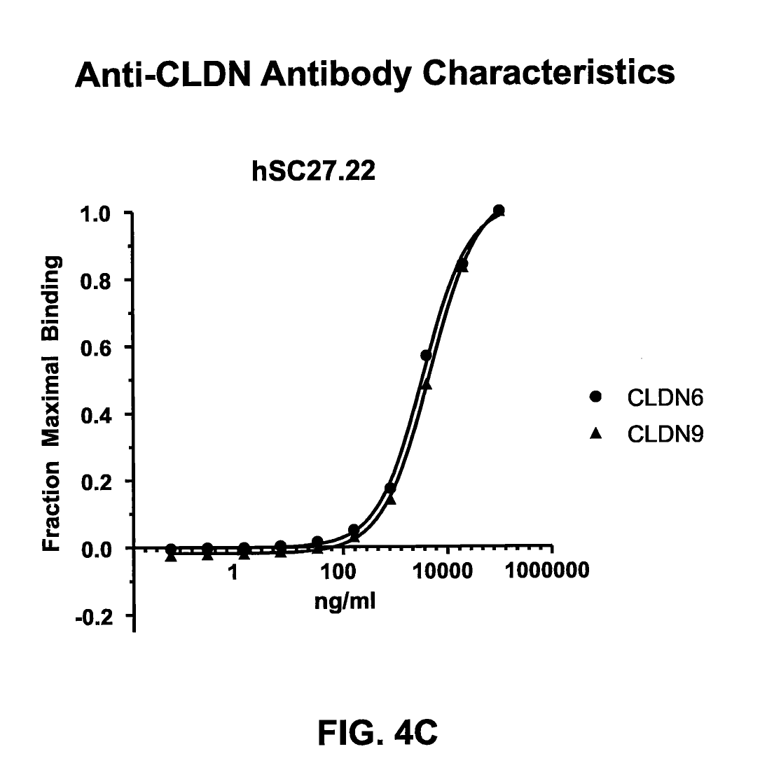

The flow cytometry assay described above resulted in the identification of numerous anti-CLDN antibodies. Cross reactivity was determined based on the change in geometric mean fluorescence intensity (ΔMFI) for the binding of the antibody to the cell lines specifically overexpressing the indicated CLDN family member versus the signal determined using a fluorescence minus one (FMO) isotype-control (gray-fill) (FIG. 4A). Thus, the two hCLDN6-binding antibodies SC27.1 and SC27.22 can be described as claudin multireactive antibodies since they cross react in this assay with three members of the human CLDN family: hCLDN6, hCLDN4 and hCLDN9. SC27.1 and SC27.22 antibodies also bound to mouse and rat orthologs of CLDN4 and CLDN9 (data not shown).

To test the ability of various additional mouse antibodies to bind to CLDN family members, flow cytometry was performed using cell lines overexpressing human CLDN4, CLDN6 or CLND9 that had been incubated with 10 μg/mL of purified primary anti-CLDN antibody, or a mouse IgG2b control antibody, followed by incubation with an Alexa 647 anti-mouse secondary antibody. As shown in FIG. 4B, all the antibodies bound to CLDN6, whereas some were CLDN6-specific (e.g. SC27.102, SC27.105, and SC27.108), and others were multireactive and bound to both CLDN6 and CLDN9 (e.g., SC27.103 and SC27.204), or to CLDN6 and CLDN4 (e.g., SC27.104). Thus a wide range of multireactive binding profiles was obtained for the antibodies of the invention.

To compare the apparent binding affinity of the multireactive anti-CLDN antibodies for CLDN6 and CLDN9, flow cytometry was performed with a serial dilution of humanized anti-CLDN antibody hSC27.22. The antibody was serially diluted to concentrations ranging from 50 pg/ml to 100 μg/ml and was added to a 96 well plate containing HEK-293T cells overexpressing CLDN6 or CLDN9, and kept on ice for one hour. A secondary anti-human antibody (Jackson ImmunoResearch Cat. # 109-605-098) was added and incubated for one hour in the dark. The cells were washed twice in PBS after which Fixable Viability Dye (eBioscience Cat # 65-0863-14) was added for 10 minutes. Following additional washing with PBS, cells were fixed with paraformaldehyde (PFA) and read on a BD FACS Canto II flow cytometer in accordance with the manufacturer's instructions. MFI values were normalized using fluorescent microspheres (Bangs Laboratories) according to manufacturer's instructions. Normalized maximal MFI values observed for the binding of the antibody to either CLDN6 or CLDN9 expressing cells were used to transform the data into fraction maximal binding for each overexpressing cell, using the equation: fraction maximal binding=(observed normalized MFI/maximal normalized MFI). Apparent EC50 values for the binding of hSC27.22 to each cell line were then calculated using a four parameter variable slope curve fitting for a log (inhibitor) vs. response model supplied in the GraphPad Prism software package (La Jolla, Calif.). FIG. 4C shows that the humanized multireactive anti-CLDN6 antibody, hSC27.22, has an apparent EC50 for CLDN6 which is substantially the same as that for CLDN9. (apparent EC50 CLDN6—3.45 μg/mL (r² for goodness of fit=0.9987, 99% confidence bounds: 2.51-4.75 μg/mL); apparent EC50 CLDN9 —4.66 μg/mL (r² for goodness of fit=0.9998, 99% confidence bounds: 4.09-5.31 μg/mL)).

Accordingly, the binding domains of the disclosed CARs may be tailored to associate with selected CLDN proteins (e.g., CLDN6) or with combinations of CLDN proteins (e.g., CLDN6 and CLDN9) depending on the desired therapeutic index.

### Example 7

### Generation of an Anti-CLDN Chimeric Antigen Receptors

Fabrication of an Anti-CD19 CAR

To generate a positive control CAR construct, a synthetic open reading frame encoding a second generation CAR directed towards human CD19 (see US2014/0271635) was synthesized (Life Technologies) and subcloned into the multiple cloning site (MCS) of the lentiviral expression vector pCDH-CMV-MCS-EF1-GFP-T2A-Puro (System Biosciences, Mountain View Calif.). The expression of the CD19 CAR construct is then driven by the CMV promoter, while the bicistronic GFP-T2A-Puro open reading frame permits detection of transduced cells by analysis of GFP expression (e.g., microscopy or FACS) and selection of cells using the antibiotic puromycin. The anti-CD19 CAR open reading frame comprises nucleotides, from 5′ to 3′, encoding the signal leader sequence from the human CD8 alpha chain (amino acids 1-21, UniProt accession P01732-1), a scFv derived from a mouse monoclonal antibody recognizing human CD19 (Nicholson et al, 1997; PMID 9566763), the human CD8 alpha hinge, transmembrane domain and proximal region (amino acids 138-206, UniProt accession P01732-1), the intracellular co-stimulatory signaling region from the human 4-1BB protein (amino acids 214-255, UniProt accession Q07011-1), and the human CD3_{ζ} chain intracellular signaling region (amino acids 52-164, UniProt accession P20963-1). Besides providing a positive control the anti-CD19 CAR/lentiviral expression vector was designed with restriction sites in such a way that the anti-CD19 scFv component could be easily removed and substituted with an alternative binding region component directed to any selected determinant. As described below, this cassette system shown in FIG. 5 (SCT1-XX where XX indicates the particular CLDN binding domain component) was used to validate various embodiments of the instant invention. Note that the SCT nomenclature may, depending on the context, refer to the expressed anti-CLDN CAR protein, cytotoxic lymphocytes expressing the CAR protein, the anti-CLDN CAR ORF or an expression vector (e.g., lentiviral, retroviral, plasmid, etc.) comprising the same ORF depending on the context.

Fabrication of SCT1-h27.108.

To generate a novel anti-CLDN CAR construct (SCT1-h27.108), a nucleotide sequence encoding an scFv fragment was first synthesized by operably linking anti-hSCh27.108 VL (SEQ ID NO. 68) and VH (SEQ ID NO. 70) nucleotide sequences together via a pentameric multimer GlyGlyGlyGlySer (G₄S)₃ (GGGGSGGGGSGGGGS; SEQ ID NO. 3) linker to provide a hSC27.108-scFv polynucleotide sequence:

(SEQ ID NO. 4)
GAAATCGTGCTTACACAATCCCCTGCCACTCTGAGCCTTTCTCCAGGCGA
GCGAGCAACCCTTTCCTGCAGTGTTTCCTCTTCAATCAGTTCCAGCAATT
TGCACTGGTACCAGCAGAAGCCTGGTCAGGCACCCCGATTGTTGATCTAT
GGCACATCTAACCTGGCCAGCGGCATCCCTGCTCGGTTCAGTGGATCTGG
CTCCGGAACAGATTTCACTCTCACTATCAGCTCCCTTGAGCCTGAAGATT
TTGCCGTGTACTACTGTCAGCAATGGAGTTCCTACCCCCACACCTTTGGC
GGCGGGACAAAGGTCGAGATAAAAGGCGGCGGAGGATCTGGCGGAGGCGG
AAGTGGCGGAGGGGGATCTCAGGTACAGCTGGTCCAGTCCGGCGCTGAGG
TTAAGAAGCCCGGTGCCTCCGTGAAGGTATCTTGTAAGGCCTCAGGTTAC
ACCTTTACAAATTATCTGATCGAATGGGTGAGACAGGCCCCAGGTCAGGG
TCTGGAATGGATGGGACTCATCAACCCTGGGAGTGGCGGGACCAACTACA
ACGAAAAGTTTAAGGGGAGAGTGACAATGACCACAGATACCAGTACCTCC
ACCGCATATATGGAGCTGCGAAGCTTGAGGTCCGATGACACTGCTGTGTA
CTATTGCGCCCGTAGAAGCCCACTCGGGTCTTGGATCTATTACGCATACG
ATGGTGTGGCCTATTGGGGCCAGGGCACCCTGGTGACAGTCTCCTCG

that encodes the amino acid sequence set forth immediately below:

(SEQ ID NO. 5)
EIVLTQSPATLSLSPGERATLSCSVSSSISSSNLHWYQQKPGQAPRLLIY
GTSNLASGIPARFSGSGSGTDFTLTISSLEPEDFAVYYCQQWSSYPHTFG
GGTKVEIKGGGGSGGGGSGGGGSQVQLVQSGAEVKKPGASVKVSCKASGY
TFTNYLIEWVRQAPGQGLEWMGLINPGSGGTNYNEKFKGRVTMTTDTSTS
TAYMELRSLRSDDTAVYYCARRSPLGSWIYYAYDGVAYWGQGTLVTVSS

wherein the scFv comprises the VL amino acid sequence set forth in SEQ ID NO. 69 and the VH amino acid sequence set forth in SEQ ID NO. 71.

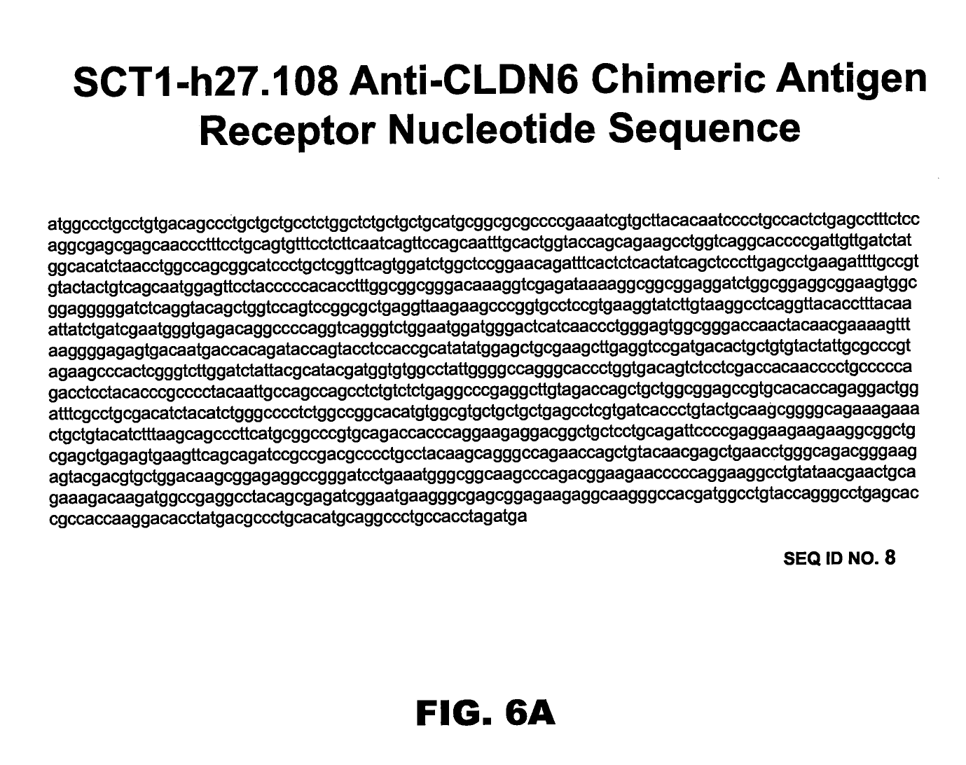

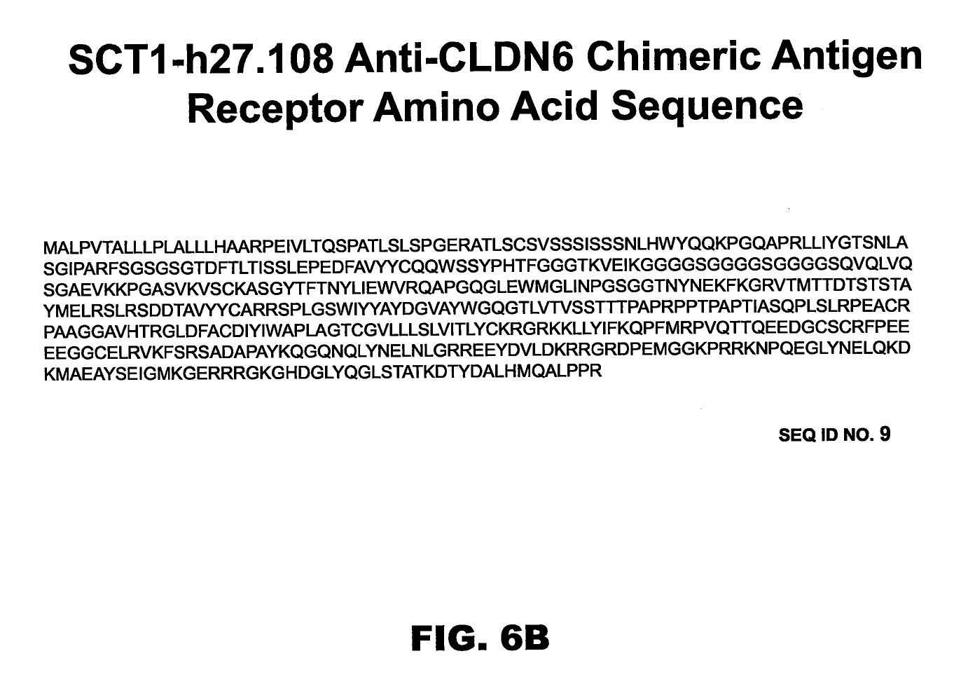

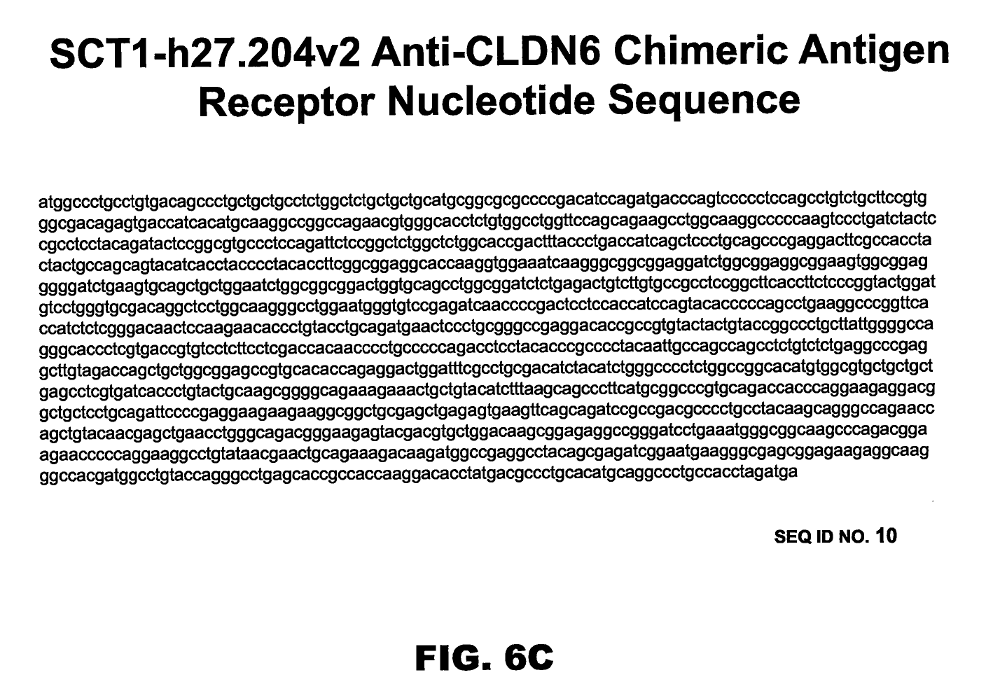

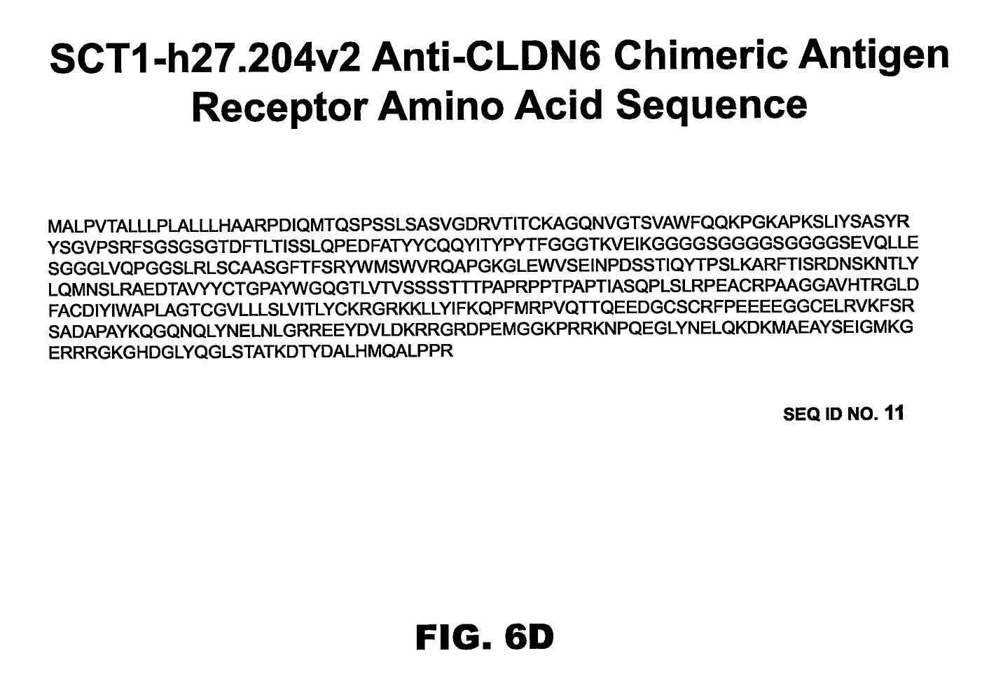

Using standard molecular engineering techniques the hSC27.108-scFv nucleotide sequence was subsequently cloned into the SCT1 cassette to provide a SCT1-h27.108 lentiviral expression vector comprising an anti-CLDN CAR. In this regard the SCT1-27.108 CAR comprises an open reading frame encoding the following elements from 5′ to 3′: CD8 alpha chain leader region (amino acids 1-21, UniProt P01732-1), h27.108 VL domain (as per Example 5), (G₄S)₃ synthetic linker sequence (amino acid 1-15, Huston et al., 1988), h27.108 VH domain (as per Example 5), the human CD8 alpha hinge and transmembrane domain (amino acids 138-206, UniProt accession P01732-1), the intracellular co-stimulatory signaling region from the human 4-1BB protein (amino acids 214-255, UniProt accession Q07011-1) and the human CD3_{ζ} chain intracellular signaling region (amino acids 52-164, UniProt accession P20963-1). The CAR open reading frame was sequence confirmed. A schematic diagram of the SCT1-h27.108 CAR open reading frame is set forth in FIG. 5 with the corresponding nucleic acid sequence set forth in FIG. 6A (SEQ ID NO. 8) and the resulting amino acid sequence set forth in FIG. 6B (SEQ ID NO. 9).

Fabrication of SCT1-h27.204 v2.

To generate a novel anti-CLDN CAR construct (SCT1-h27.204v2), a nucleotide sequence encoding an scFv fragment was first synthesized by operably linking anti-hSCh27.204 VL (SEQ ID NO. 72) and VH (SEQ ID NO. 86) nucleotide sequences together via a pentameric multimer GlyGlyGlyGlySer (G₄S)₃ (GGGGSGGGGSGGGGS; SEQ ID NO. 3) linker to provide a hSC27.204v2-scFv polynucleotide sequence:

(SEQ ID NO. 6)
GACATCCAGATGACCCAGTCCCCCTCCAGCCTGTCTGCTTCCGTGGGCGA
CAGAGTGACCATCACATGCAAGGCCGGCCAGAACGTGGGCACCTCTGTGG
CCTGGTTCCAGCAGAAGCCTGGCAAGGCCCCCAAGTCCCTGATCTACTCC
GCCTCCTACAGATACTCCGGCGTGCCCTCCAGATTCTCCGGCTCTGGCTC
TGGCACCGACTTTACCCTGACCATCAGCTCCCTGCAGCCCGAGGACTTCG
CCACCTACTACTGCCAGCAGTACATCACCTACCCCTACACCTTCGGCGGA
GGCACCAAGGTGGAAATCAAGGGCGGCGGAGGATCTGGCGGAGGCGGAAG
TGGCGGAGGGGGATCTGAAGTGCAGCTGCTGGAATCTGGCGGCGGACTGG
TGCAGCCTGGCGGATCTCTGAGACTGTCTTGTGCCGCCTCCGGCTTCACC
TTCTCCCGGTACTGGATGTCCTGGGTGCGACAGGCTCCTGGCAAGGGCCT
GGAATGGGTGTCCGAGATCAACCCCGACTCCTCCACCATCCAGTACACCC
CCAGCCTGAAGGCCCGGTTCACCATCTCTCGGGACAACTCCAAGAACACC
CTGTACCTGCAGATGAACTCCCTGCGGGCCGAGGACACCGCCGTGTACTA
CTGTACCGGCCCTGCTTATTGGGGCCAGGGCACCCTCGTGACCGTGTCCT
CT

that encodes the amino acid sequence set forth immediately below:

(SEQ ID NO. 7)
DIQMTQSPSSLSASVGDRVTITCKAGQNVGTSVAWFQQKPGKAPKSLIY
SASYRYSGVPSRFSGSGSGTDFTLTISSLQPEDFATYYCQQYITYPYTF
GGGTKVEIKGGGGSGGGGSGGGGSEVQLLESGGGLVQPGGSLRLSCAAS
GFTFSRYWMSWVRQAPGKGLEWVSEINPDSSTIQYTPSLKARFTISRDN
SKNTLYLQMNSLRAEDTAVYYCTGPAYWGQGTLVTVSS

wherein the scFv comprises the VL amino acid sequence set forth in SEQ ID NO. 73 and the VH amino acid sequence set forth in SEQ ID NO. 87. Using standard molecular engineering techniques the hSC27.204v2-scFv nucleotide sequence was subsequently cloned into the SCT1 cassette to provide a SCT1-h27.204v2 lentiviral expression vector comprising an anti-CLDN CAR. In this regard the SCT1-27. 204v2 CAR comprises an open reading frame encoding the following elements from 5′ to 3′: CD8 alpha chain leader region (amino acids 1-21, UniProt P01732-1), h27.204 VL domain (as per Example 5), (G₄S)₃ synthetic linker sequence (amino acid 1-15, Huston et al., 1988), h27. 204v2 VH domain (as per Example 5), the human CD8 alpha hinge and transmembrane domain (amino acids 138-206, UniProt accession P01732-1), the intracellular co-stimulatory signaling region from the human 4-1BB protein (amino acids 214-255, UniProt accession Q07011-1) and the human CD3_{ζ} chain intracellular signaling region (amino acids 52-164, UniProt accession P20963-1). The CAR open reading frame was sequence confirmed. A schematic diagram of the SCT1-h27.204v2 CAR open reading frame is set forth in FIG. 5 with the corresponding nucleic acid sequence set forth in FIG. 6C (SEQ ID NO. 10) and the resulting amino acid sequence set forth in FIG. 6D (SEQ ID NO. 11).

### Example 8

### Generation and Characterization of Lentiviral Vector Particles

Lentiviral vector packaging of SCT1-h27.108 and SCT1-h27.204v2 were carried out as follows: 10 ug of SCT1-h27.108 or SCT1-h27.204v2 plasmid, 7 ug of pΔR8.74, and 4 ug of pMD2.G were co-transfected into ten-million HEK-293T cells (ATCC) in the presence of polyethylenemine (Polysciences) at a DNA:PEI ratio of 1:4. Co-transfected cells were incubated at 37° C. (5%CO₂) overnight, followed by media exchange the next day. Forty-eight hours post-transfection, culture media containing lentiviral particles was harvested and clarified by centrifugation at 1200 rpm for 5 min at 4° C. to remove cell debris. To pellet lentiviral vector particles the clarified culture media was ultracentrifuged at 19500 rpm for two hours at 4° C. After ultracentrifugation the supernatant was discarded, the viral pellet resuspended in sterile PBS, and stored at −80° C. Quantitation of recovered lentiviral vector stocks was assessed by p24 ELISA (Cell Biolabs), and gene-transfer efficiency (functional titer) was assessed by standard lentiviral vector titration methods. Typical yields of lentiviral vector stocks ranged from 7-15 ug/ml of p24 antigen, and functional titers ranged from 1-3×10e8 TU/ml. The SCT1-h27.108 and SCT1-27.204v2 lentiviral vector stocks were frozen and stored until use.

As set forth in the subsequent Examples the vector stocks may be used to generate sensitized lymphocytes and induce a desired immune response as discussed in detail throughout the instant application and shown schematically in FIG. 7 appended hereto.

### Example 9

### Generation of T Lymphocytes Expressing SCT1-h27.108 and SCT1-h27.204v2 CAR Constructs

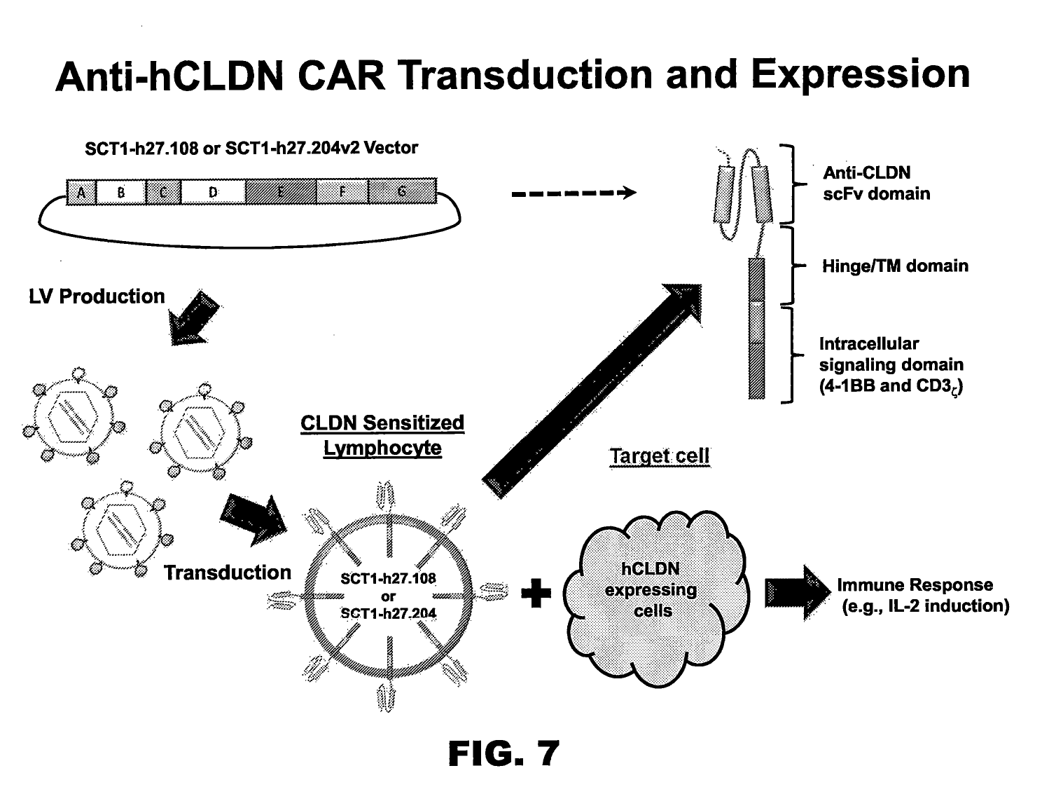

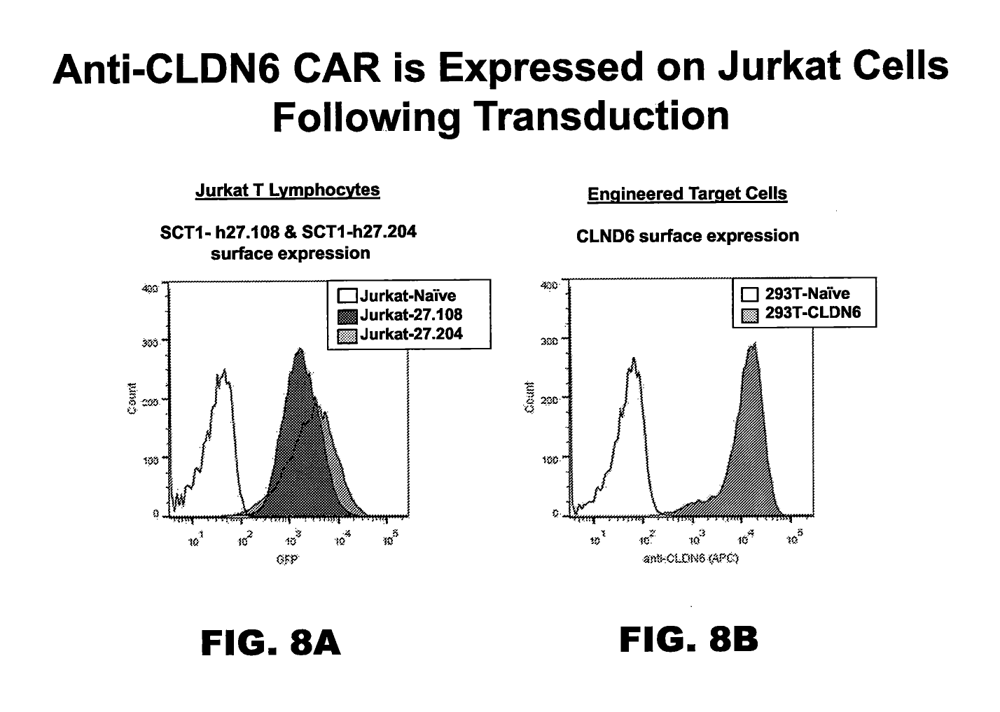

CLDN target-specific Jurkat lymphocytes expressing SCT1-h27.108 or SCT1-h27.204v2 were generated by transducing one million Jurkat E6-1 (ATCC) T lymphocyte cells with the SCT1-h27.108 or SCT1-h27.204v2 lentiviral vector from the previous Example at a multiplicity of infection (MOI) of ˜4 in the presence of 10 ug/ml of polybrene (EMD Millipore) to ensure efficient viral transduction. The cells were allowed to incubate in the presence of lentiviral particles for seventy-two hours at 37° C. (5%CO₂). Afterwards, the spent media was exchanged with fresh media containing 2 ug/ml Puromcyin (Life Technologies) to positively select for SCT1-h27.108 or SCT1-h27.204v2-expressing cells. Cells were allowed to incubate an additional 5 days in the presence of Puromycin prior to inferring the presence of anti-CLDN scFv on the cell surface by flow cytometry (FIG. 8A). More particularly, the transduced Jurkat cells expressing SCT1-h27.108 or SCT1-h27.204v2 and non-transduced control cells were then pelleted, washed and resuspended in buffer as described herein. The preparations were then analyzed on a BD FACS Canto II flow cytometer as per the manufacturer's instructions to detect the presence of GFP, and hence infer expression of the SCT1-h27.108 or SCT1-h27.204, as evidenced in FIG. 8A.

Flow cytometry was also used to detect the presence of hCLDN6 protein on the surface of an engineered HEK-293T cell line overexpressing hCLDN6 (FIG. 8B) that are used to characterize CAR constructs of the instant invention. In this regard HEK-293T parental cells or HEK-293T cells overexpressing hCLDN6 were harvested and isolated into single cell suspensions with Versene (Life Technologies). The isolated cells were washed as described above and incubated for 30 minutes at 4° C. in the dark with 1 microgram of anti-CLDN6 antibody prior to thrice washing in PBS/2% FCS. The cells were then incubated for 30 minutes with 50 μL per sample AlexaFluor-647 labeled goat-anti-mouse IgG, Fc fragment specific secondary antibody (Life Technologies) diluted 1:200 in PBS/2%FCS, washed thrice with PBS/2% FCS and re-suspended in PBS/2% FCS with DAPI (to detect living cells). The cells were then analyzed on a BD FACS Canto II flow cytometer as per the manufacturer's instructions to provide the data set forth in FIG. 8B.

FIGS. 8A and 8B, respectively, demonstrate that the SCT1-h27.108 and SCT1-h27.204v2 are expressed on transduced Jurkat T lymphocytes but not on non-transduced Jurkat cells, and that human CLDN6 protein is expressed on the engineered HEK-293T cells but not on HEK-293T-Naïve cells.

### Example 10

### Jurkat-SCT1-h27.108 or SCT1-h27.204v2 T Lymphocytes Induce IL-2 Production upon Contacting hCLDN Expressing Cells

Transduced Jurkat-SCT1-h27.108 or SCT1-h27.204v2 lymphocytes were assessed for target-specific activity by measuring IL-2 induction, which is indicative of CAR mediated T-cell activation. More specifically, using transduced Jurkat lymphocytes and engineered 293T cells expressing hCLDN6 from the previous Example, IL-2 levels were monitored to demonstrate that the CAR expressing lymphocytes are activated and mount an immune response upon contact with cells expressing hCLDN6.

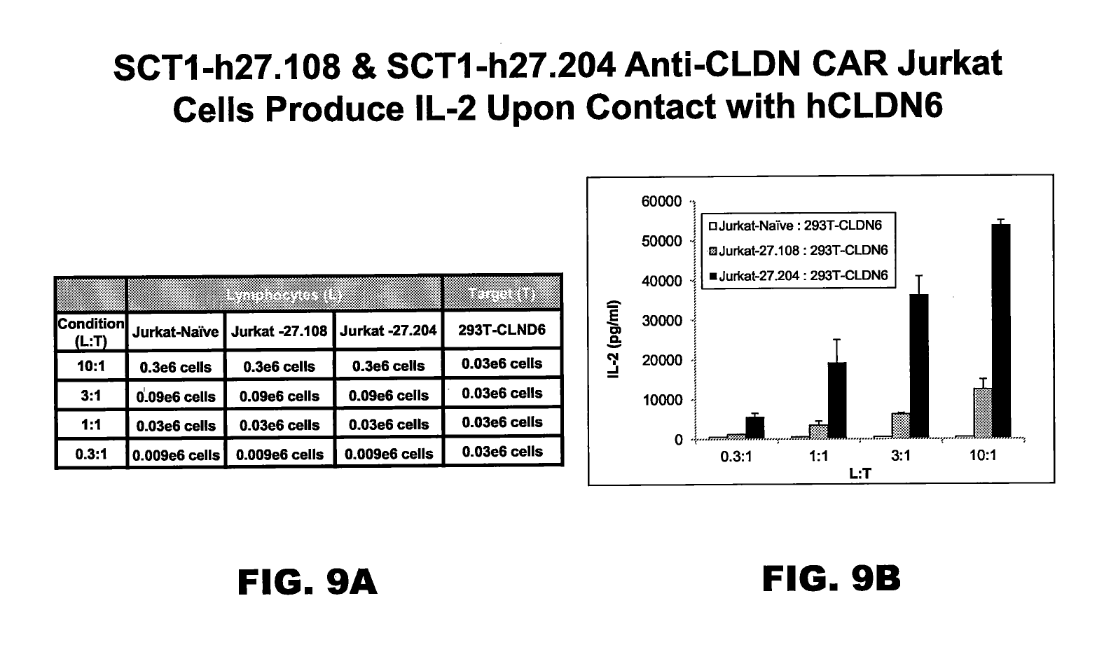

In this regard Jurkat-SCT1-h27.108 or SCT1-h27.204v2 lymphocytes from Example 9 were co-cultured with HEK-293T cells engineered to over-express hCLDN6 antigen on the cell surface as evidenced by flow cytometry. Co-culturing of lymphocytes with target HEK-293T-hCLDN cells was performed at the described lymphocyte to target cell (L:T) ratios set forth in FIG. 9A to assess dose response and determine maximum IL-2 production conditions. Co-cultures were incubated at 37° C. (5%CO₂) for 48 hrs, at which time media was harvested and clarified of cell debris by centrifugation at 1200 rpm for 5 minutes. Clarified supernatant was then assessed for IL-2 production by ELISA (Thermo Scientific) per manufacturer's instructions. To assess background IL-2 production, non-transduced Jurkat cells (Jurkat-Naïve) were co-cultured with HEK-293T-hCLDN cells.

As evidenced by the data set forth in FIG. 9B, the Jurkat-SCT1-h27.108 or SCT1-h27.204v2 lymphocytes were prompted to produce IL-2 in a concentration dependent manner upon exposure to cells expressing hCLDN6. Such IL-2 production is indicative of T-cell activation by the SCT1 CAR upon recognition of CLDN antigen on hCLDN6 expressing cells (including hCLDN expressing tumorigenic cells). Specific CAR-mediated activation of Jurkat cells is further elucidated by the lack of observable IL-2 production among co-cultures containing HEK-293T-CLDN and non-transduced Jurkat cells (FIG. 9B).

As previously alluded to the process for generating this desired immune response, as discussed in detail throughout the instant application, is shown schematically in FIG. 7 appended hereto.

### Example 11

### Generation of T Lymphocytes Expressing SCT1-h27.108 or SCT1-h27.204v2 CAR Constructs

In order to demonstrate that the disclosed CARs may be used to provide sensitized lymphocytes primary human CD3+ T lymphocytes were isolated from commercially available peripheral blood mononuclear cell preparations (PBMCs: AllCells) using a human CD3 positive selection kit (Stemcell Technologies). The PBMCs were obtained from two different donors (donor 1 and donor 2).

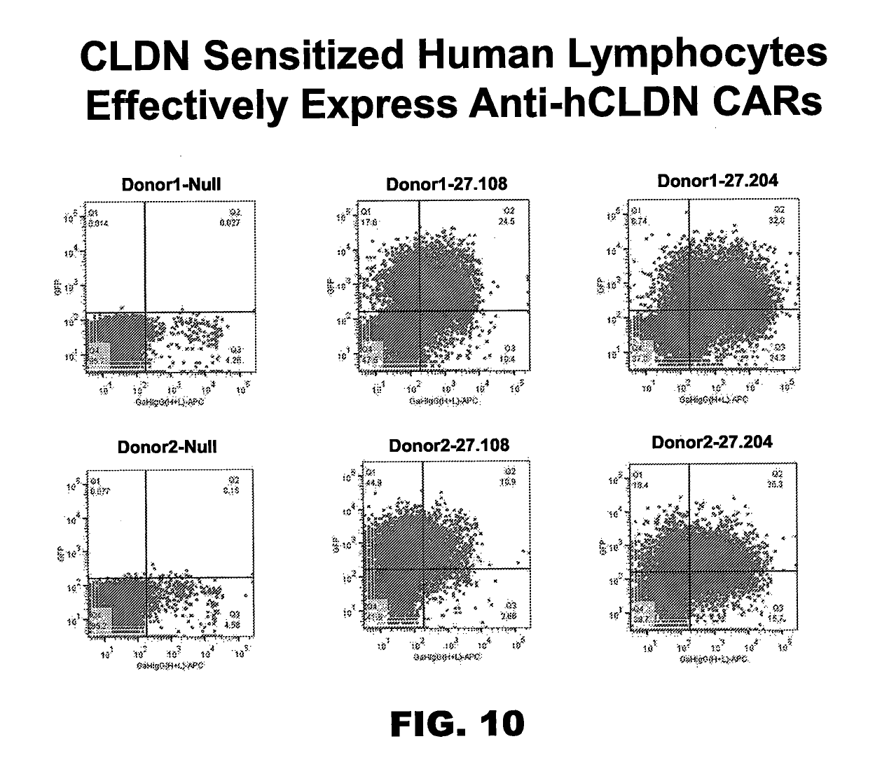

Following isolation CD3+ T cells were cultured in RPMI media containing 10% heat-inactivated fetal bovine serum (Hyclone), 1% penicillin/streptomycin (Corning), 1% 1-glutamine (Corning), and 10 mM HEPES (Corning). T lymphocytes were incubated at 37° C. (5%CO₂) in the presence of CD3/CD28 activation beads (Dynabeads) at a 1:5 ratio for activation. IL-2 (Peprotech) was added every other day to a final concentration of 50 IU/ml. Twenty-four hours after initial activation, CLDN target-specific T lymphocytes expressing SCT1-h27.108 or SCT1-h27.204v2 were generated by transducing one million T cells with SCT1-h27.108 or SCT1-h27.204v2 lentiviral vectors (generated substantially as set forth in Example 8) at a multiplicity of infection (MOI) of ˜5 in the presence of 10 ug/ml of polybrene (EMD Millipore) to ensure efficient viral transduction. The cells were allowed to incubate in the presence of lentiviral particles for seventy-two hours at 37° C. (5%CO₂) prior to assessing the anti-CLDN CAR surface expression by flow cytometry (FIG. 10).

Flow cytometry analysis of transduced T lymphocytes expressing SCT1-h27.108 or SCT1-h27.204v2 along with non-CAR-bearing T lymphocyte control cells was performed as follows: 10⁶ cells of each sample were harvested and pelleted by centrifugation at 1200 rpm at 4° C. for 5 minutes; supernatant was removed and the pellet was washed in cold PBS/2% FCS twice. After supernatant from the final wash was removed, in order to directly detect the presence of the CAR at the surface of the cells, the cell pellet was resuspended in 100 microliters of PBS/2% FCS containing 1 microgram of Alexa Fluor® 647-conjugated Affinipure Goat Anti-Human IgG, F(ab′) antibody (Jackson ImmunoResearch). The cells were incubated in the dark at 4° C. for 30 minutes. After incubation, cells were washed thrice in PBS/2% FCS before being re-suspended in PBS/2% FCS with DAPI (to detect living cells). The cells were then analyzed on a BD FACS Canto II flow cytometer as per the manufacturer's instructions to provide the data set forth in FIG. 10.

FIG. 10 clearly shows that SCT1-h27.108 or SCT1-h27.204v2 are both expressed on transduced primary T lymphocytes (i.e., sensitized lymphocytes) but not on non-transduced lymphocytes.

### Example 12

### Generation and Characterization of Cells Expressing CLDN Target Antigen

As set forth in Example 9 flow cytometry was used to detect the presence of CLDN protein on the surface of an engineered HEK-293T cell line overexpressing human CLDN6. Similarly flow cytometry was used to confirm the expression of human CLDN on a patient-derived xenograph (PDX) tumor cell line (OV78). Both the artificially engineered 293T cell line and the derived ovarian cancer cell line may be used to characterize sensitized lymphocytes of the instant invention.

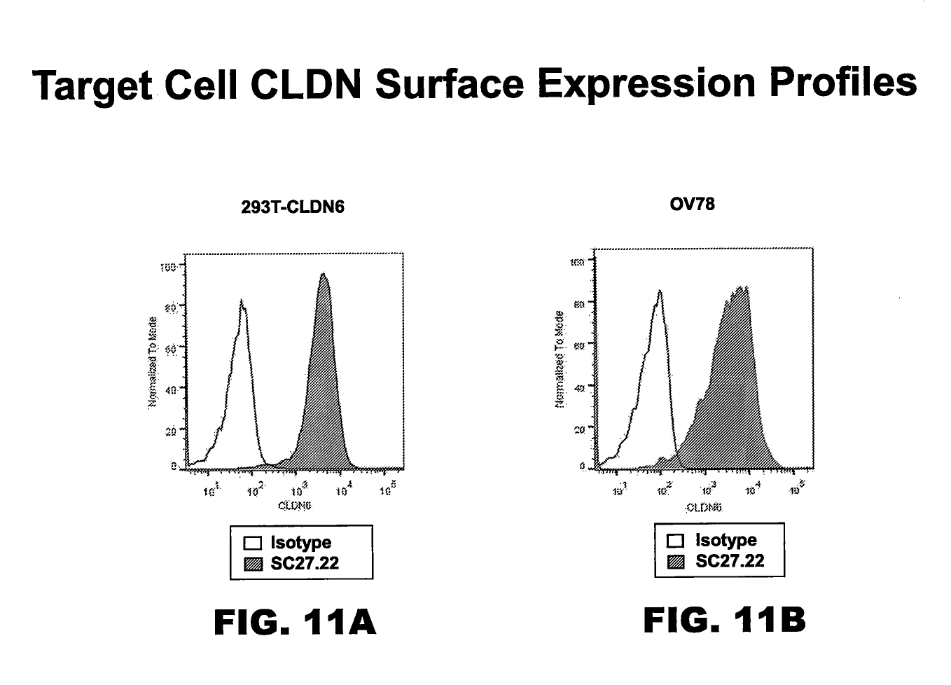

More particularly HEK-293T cells overexpressing human CLDN6 (293T-CLDN) were harvested and isolated into single cell suspensions with Versene (Life Technologies). Similarly, freshly harvested OV78 PDX tumors were processed into single cell suspension using a tumor dissociation kit (Mylteni Biotec). Isolated cells were washed as described herein and incubated for 30 minutes at 4° C. in the dark with 1 microgram of anti-CLDN antibody (SC27.22) or isotype control prior to washing three times in PBS/2% FCS. The cells were then incubated for 30 minutes with 50 microliters per sample of AlexaFluor-647 labeled goat-anti-mouse IgG, Fc fragment specific secondary antibody (Life Technologies) diluted 1:200 in PBS/2%FCS, washed thrice with PBS/2% FCS and resuspended in PBS/2% FCS with DAPI (to detect living cells). Cells were then analyzed on a BD FACS Canto II flow cytometer as per the manufacturer's instructions to provide the data set forth in FIGS. 11A and 11B.

The resulting data show that human CLDN protein is expressed both on the engineered HEK-293T cells (FIG. 11A) and OV78 PDX tumor cells (FIG. 11B).

### Example 13

### T Lymphocyte-SCT1-h27.108 or SCT1-h27.204v2 Induce Cytokine Production upon Contacting CLDN-Expressing Cells

Sensitized lymphocytes comprising SCT1-h27.108 or SCT1-h27.204v2 were assessed for target-specific activation by measuring IFNγ and TNFα induction upon contact with CLDN-expressing target cells. It will be appreciated that cytokine production (e.g., TNFα and IFNγ induction) is indicative of active chimeric antigen receptors that are capable of inducing an anti-tumor immune response.

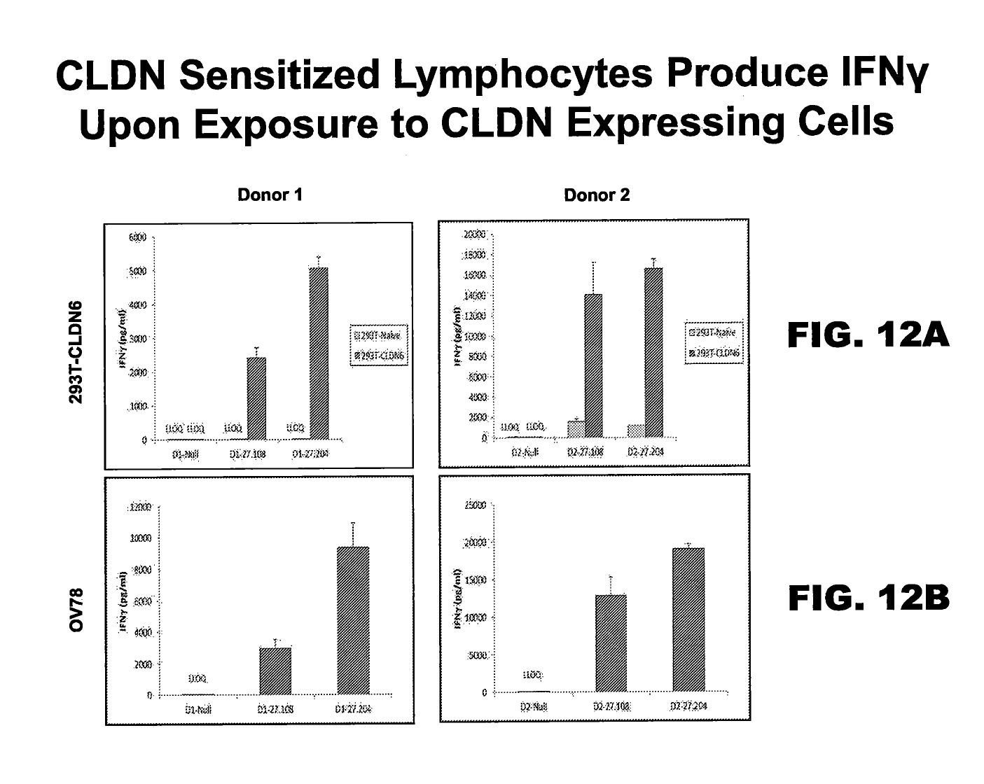

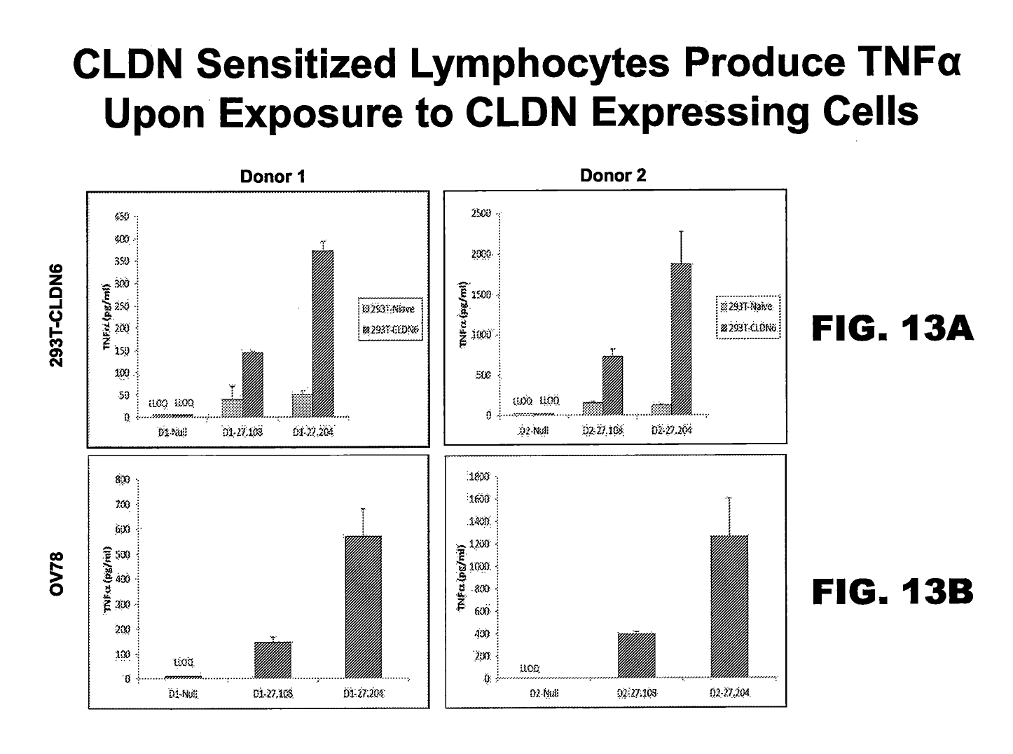

To assess target-specific (i.e. CLDN) activation sensitized lymphocytes comprising host cells from two different donors were exposed to CLDN+ 293T cells and ovarian cancer cells expressing CLDN (both from Example 11). More specifically, PMBC preparations from two different donors (donor 1 and donor 2) were used to provide CD3+ T lymphocyte preparations substantially as set forth above. The respective lymphocyte preparations were then transduced with SCT1-h27.108 or SCT1-h27.204v2 substantially as set forth in Example 11 to provide donor 1 and donor 2 CLDN sensitized lymphocyte preparations (along with non-transduced lymphocytes as controls). Each of the sensitized lymphocyte preparations (with controls) were then co-cultured with either 293T-CLDN or OV78 PDX target cells at an effector to target (E:T) ratio of 3:1. Co-cultures were incubated at 37° C. (5%CO₂) for 48 hrs, at which time media was harvested and clarified of cell debris by centrifugation at 1200 rpm for 5 minutes. Clarified supernatant was then assessed for TNFα production by ELISA (Thermo Fisher) and IFNγ by ELISA (Invitrogen) per manufacturer's instructions. The resulting measurements for levels of IFNγ and TNFα are shown, respectively, in FIGS. 12A and 12B (IFNγ) and FIGS. 13A and 13B (TNFα). In both instances, higher cytokine production is indicative of more robust activation of CAR+ populations.

As evidenced by the data set forth in FIGS. 12A (293 cells) and 12B (tumor cells) and FIGS. 13A (293 cells) and 13B (tumor cells) the SCT1-h27.108 or SCT1-h27.204v2-bearing T lymphocytes were prompted to produce TNFα and IFNγ upon exposure to both engineered and tumor-derived cells expressing human CLDN, whereas the non-CAR-bearing T lymphocytes exhibited minimal TNFα and IFNγ induction when co-cultured with the same target cells. This confirms that the CLDN sensitized lymphocytes of the instant invention are active and capable of generating immunostimulatory signals upon exposure to CLDN+ tumor cells.

### Example 14

### Targeted Killing of CLDN-Expressing Cells In Vitro by SCT1-h27.108 or SCT1-h27.204v2-T lymphocytes

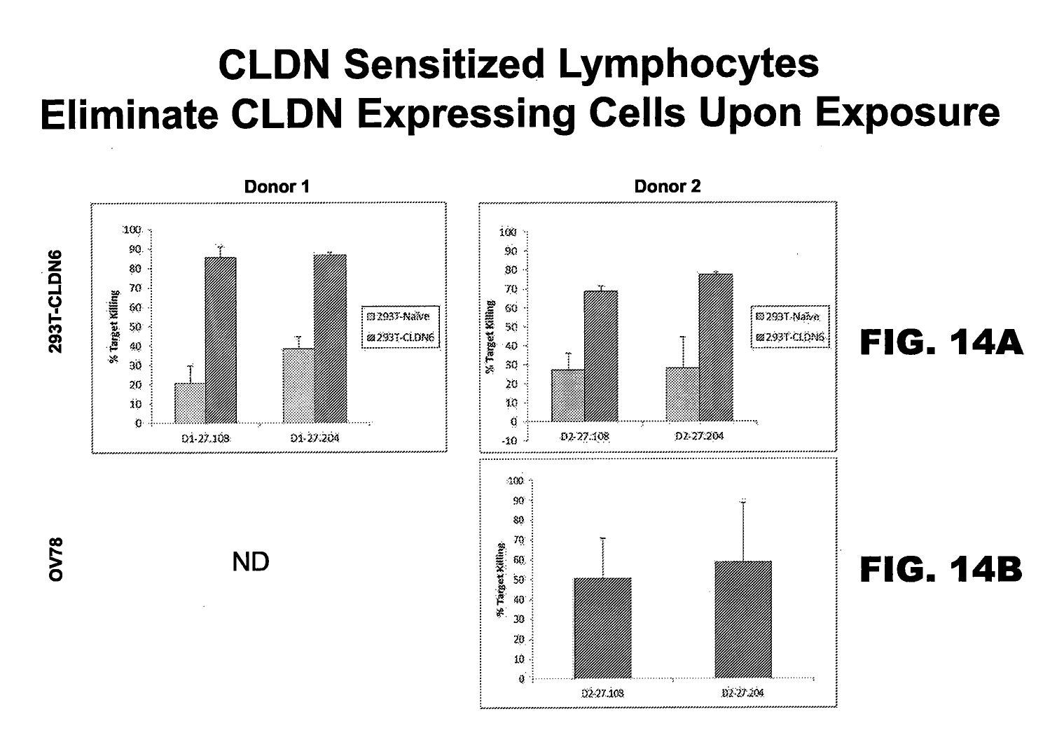

To demonstrate the ability of CLDN sensitized lymphocytes to kill cells in a target-specific manner, CAR transduced cells of the instant invention were exposed to engineered 293 cells and tumor cells expressing CLDN (each from Example 12). Following exposure the number of living target cells remaining was calculated with the results being set forth in FIGS. 14A (293 cells) and 14B (tumor cells).

More particularly SCT1-h27.108 or SCT1-h27.204v2 sensitized lymphocytes (prepared as per Example 11 with host cells from two donors) were co-cultured with either 293T-CLDN or OV78 PDX cells at an effector to target (E:T) ratio of 3:1 (Note that due to assay conditions measurements of the activity of donor 1 lymphocytes exposed to OV78 cells was inconclusive). Co-cultures were incubated at 37° C. (5%CO₂) for 48 hrs prior to determination of remaining viable CLDN-bearing cells.

The percentage of live cells was calculated as follows: co-cultures were harvested and washed as set forth herein prior to incubation for 30 minutes at 4° C. in the dark with 1 microgram of anti-CLDN antibody or isotype control followed by washing three times in PBS/2% FCS. Cells were then incubated for 30 minutes with 50 microliters per sample of AlexaFluor-647 labeled goat-anti-mouse IgG Fc fragment-specific secondary antibody (Life Technologies) diluted 1:200 in PBS/2%FCS. Cells were washed three times with PBS/2% FCS, followed by resuspension in 200 microliters of PBS/2% FCS containing DAPI (Life Technologies) for cell viability discrimination, and 10000 absolute counting beads (Life Technologies) for normalizing cell counts. Analysis and enumeration of remaining viable CLDN-bearing target cells was performed on a BD FACS Canto II flow cytometer by quantifying the respective number of viable CLDN-bearing cells per 7500 absolute counting beads collected. The number of viable target cells remaining in presence of non-CAR-bearing T lymphocytes was used as the benchmark to compare target-specific killing of CLDN-bearing cells by SCT1-h27.108 or SCT1-h27.204v2 sensitized lymphocytes.

As shown in FIGS. 14A (293 cells) and 14B (tumor cells) 293T-CLDN6 cells exhibited significant susceptibility to cytolysis by either SCT1-h27.108-T lymphocytes or SCT1-h27.204-T lymphocytes, with greater than 70% of target cells being eliminated. Importantly, similar results were demonstrated when anti-CLDN6 CAR-bearing-T lymphocytes were cultured in presence of OV78 PDX tumor cells, wherein approximately 50% of OV78 cells were eliminated. Overall, these data demonstrate activation and killing of CLDN6-expressing cells through CLDN6-specific recognition by SCT1-h27.108 and SCT-h27.204 CAR.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A chimeric antigen receptor comprising an anti-CLDN binding domain.

2. The chimeric antigen receptor of claim 1 wherein the anti-CLDN binding domain comprises a scFv anti-CLDN binding domain.

3. The chimeric antigen receptor of claim 2 wherein the scFv anti-CLDN binding domain comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59.

4. The chimeric antigen receptor of claim 3 wherein the scFv anti-CLDN binding domain comprises
(a) three complementarity determining regions of a light chain variable region (VL) of SEQ ID NO: 69 and three complementarity determining regions of a heavy chain variable region (VH) of SEQ ID NO: 71; or
(b) three complementarity determining regions of a VL of SEQ ID NO: 73 and three complementarity determining regions of a VH of SEQ ID NO: 87.

5. The chimeric antigen receptor of claim 1 wherein the binding domain immunospecifically binds to CLDN6.

6. The chimeric antigen receptor of claim 5 wherein the binding domain is not cross-reactive with CLDN4 or CLDN9.

7. The chimeric antigen receptor of claim 5 wherein the binding domain is cross-reactive with CLDN4 or CLDN9.

8. The chimeric antigen receptor of any of claims 1 to 7 comprising an intracellular domain comprising a 4-1BB signaling domain and a CD3ζ signaling domain.

9. The chimeric antigen receptor of claim 8 further comprising a transmembrane domain comprising a human CD8 alpha hinge.

10. A polynucleotide encoding a chimeric antigen receptor of any one of claims 1 to 9.

11. A vector comprising a polynucleotide of claim 10.

12. The vector of claim 11 wherein the vector comprises a viral vector.

13. The vector of claim 12 wherein the viral vector comprises a lentiviral vector or a retroviral vector.

14. A pharmaceutical composition comprising a polynucleotide of claim 10, or a vector of any one of claims 11 to 13.

15. A kit for the preparation of CLDN sensitized lymphocytes comprising a pharmaceutical composition of claim 14.

16. An isolated host cell comprising a chimeric antigen receptor of any one of claims 1 to 9.

17. The isolated host cell of claim 16 wherein the host cell comprises a CLDN sensitized lymphocyte.

18. The isolated host cell of claim 17 wherein the CLDN sensitized lymphocyte is obtained from a patient.

19. The isolated host cell of claim 17 or 18 wherein the CLDN sensitized lymphocyte is a T cell or a NK cell.

20. The isolated host cell of claim 19 wherein the T cell is a CD8+ T cell.

21. The isolated host cell of claim 19 wherein the CLDN sensitized lymphocyte is a NK cell.

22. A pharmaceutical composition comprising a host cell of any one of claims 16 to 21.

23. A method of treating a patient suffering from cancer comprising the step of administering a pharmaceutical composition of claim 22 to the patient.

24. The method of claim 23 wherein the patient is suffering from a cancer selected from the group consisting of lung cancer, melanoma, breast cancer, prostate cancer, colon cancer, renal cell carcinoma, ovarian cancer, neuroblastoma, rhabdomyosarcoma, leukemia and lymphoma.

25. The method of claim 24 wherein the cancer is lung cancer and the lung cancer is small cell lung cancer.

26. The method of claim 24 wherein the cancer is ovarian cancer.

27. A method of reducing the frequency of cancer stem cells in a tumor cell population, wherein the method comprises contacting the tumor cell population with a pharmaceutical composition of claim 22.

28. A method of producing a CLDN-sensitized lymphocyte comprising the step of transforming a host cell with a polynucleotide of claim 10.


SEQUENCE LISTING (178 sequences)

SEQ ID NO: 1 - protein, 107 aa, Homo sapiens [sequence omitted]

SEQ ID NO: 2 - protein, 329 aa, Homo sapiens
  MISC_FEATURE: IgGI heavy chain (HC) constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 15 aa, Artificial Sequence
  scFv linker protein
GGGGSGGGGSGGGGS

SEQ ID NO: 4 - DNA, 747 bp, Artificial Sequence
  hSC27.108 scFv
gaaatcgtgcttacacaatcccctgccactctgagcctttctccaggcgagcgagcaaccctttcctgcagtgtttcctcttcaatcagttccagcaatttgcactggtaccagcagaagcctggtcaggcaccccgattgttgatctatggcacatctaacctggccagcggcatccctgctcggttcagtggatctggctccggaacagatttcactctcactatcagctcccttgagcctgaagattttgccgtgtactactgtcagcaatggagttcctacccccacacctttggcggcgggacaaaggtcgagataaaaggcggcggaggatctggcggaggcggaagtggcggagggggatctcaggtacagctggtccagtccggcgctgaggttaagaagcccggtgcctccgtgaaggtatcttgtaaggcctcaggttacacctttacaaattatctgatcgaatgggtgagacaggccccaggtcagggtctggaatggatgggactcatcaaccctgggagtggcgggaccaactacaacgaaaagtttaaggggagagtgacaatgaccacagataccagtacctccaccgcatatatggagctgcgaagcttgaggtccgatgacactgctgtgtactattgcgcccgtagaagcccactcgggtcttggatctattacgcatacgatggtgtggcctattggggccagggcaccctggtgacagtctcctcg

SEQ ID NO: 5 - protein, 249 aa, Artificial Sequence
  hSC27.108 scFv
EIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIKGGGGSGGGGSGGGGSQVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSS

SEQ ID NO: 6 - DNA, 702 bp, Artificial Sequence
  hSC27.204v2 scFv
gacatccagatgacccagtccccctccagcctgtctgcttccgtgggcgacagagtgaccatcacatgcaaggccggccagaacgtgggcacctctgtggcctggttccagcagaagcctggcaaggcccccaagtccctgatctactccgcctcctacagatactccggcgtgccctccagattctccggctctggctctggcaccgactttaccctgaccatcagctccctgcagcccgaggacttcgccacctactactgccagcagtacatcacctacccctacaccttcggcggaggcaccaaggtggaaatcaagggcggcggaggatctggcggaggcggaagtggcggagggggatctgaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 7 - protein, 234 aa, Artificial Sequence
  hSC27.204v2 scFv
DIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIKGGGGSGGGGSGGGGSEVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSS

SEQ ID NO: 8 - DNA, 1482 bp, Artificial Sequence
  SCT1-h27.108 scFv
atggccctgcctgtgacagccctgctgctgcctctggctctgctgctgcatgcggcgcgccccgaaatcgtgcttacacaatcccctgccactctgagcctttctccaggcgagcgagcaaccctttcctgcagtgtttcctcttcaatcagttccagcaatttgcactggtaccagcagaagcctggtcaggcaccccgattgttgatctatggcacatctaacctggccagcggcatccctgctcggttcagtggatctggctccggaacagatttcactctcactatcagctcccttgagcctgaagattttgccgtgtactactgtcagcaatggagttcctacccccacacctttggcggcgggacaaaggtcgagataaaaggcggcggaggatctggcggaggcggaagtggcggagggggatctcaggtacagctggtccagtccggcgctgaggttaagaagcccggtgcctccgtgaaggtatcttgtaaggcctcaggttacacctttacaaattatctgatcgaatgggtgagacaggccccaggtcagggtctggaatggatgggactcatcaaccctgggagtggcgggaccaactacaacgaaaagtttaaggggagagtgacaatgaccacagataccagtacctccaccgcatatatggagctgcgaagcttgaggtccgatgacactgctgtgtactattgcgcccgtagaagcccactcgggtcttggatctattacgcatacgatggtgtggcctattggggccagggcaccctggtgacagtctcctcgaccacaacccctgcccccagacctcctacacccgcccctacaattgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggccagaaccagctgtacaacgagctgaacctgggcagacgggaagagtacgacgtgctggacaagcggagaggccgggatcctgaaatgggcggcaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccacctagatga

SEQ ID NO: 9 - protein, 493 aa, Artificial Sequence
  SCT1-h27.108 scFv
MALPVTALLLPLALLLHAARPEIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIKGGGGSGGGGSGGGGSQVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1443 bp, Artificial Sequence
  SCT1-h27.204v2
atggccctgcctgtgacagccctgctgctgcctctggctctgctgctgcatgcggcgcgccccgacatccagatgacccagtccccctccagcctgtctgcttccgtgggcgacagagtgaccatcacatgcaaggccggccagaacgtgggcacctctgtggcctggttccagcagaagcctggcaaggcccccaagtccctgatctactccgcctcctacagatactccggcgtgccctccagattctccggctctggctctggcaccgactttaccctgaccatcagctccctgcagcccgaggacttcgccacctactactgccagcagtacatcacctacccctacaccttcggcggaggcaccaaggtggaaatcaagggcggcggaggatctggcggaggcggaagtggcggagggggatctgaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctcttcctcgaccacaacccctgcccccagacctcctacacccgcccctacaattgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggccagaaccagctgtacaacgagctgaacctgggcagacgggaagagtacgacgtgctggacaagcggagaggccgggatcctgaaatgggcggcaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccacctagatga

SEQ ID NO: 11 - protein, 480 aa, Artificial Sequence
  SCT1-h27.204v2
MALPVTALLLPLALLLHAARPDIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIKGGGGSGGGGSGGGGSEVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 8 aa, Mus musculus
RESERVED

SEQ ID NO: 13 - protein, 8 aa, Mus musculus
RESERVED

SEQ ID NO: 14 - protein, 8 aa, Mus musculus
RESERVED

SEQ ID NO: 15 - protein, 8 aa, Mus musculus
RESERVED

SEQ ID NO: 16 - protein, 8 aa, Mus musculus
RESERVED

SEQ ID NO: 17 - protein, 8 aa, Mus musculus
RESERVED

SEQ ID NO: 18 - protein, 8 aa, Mus musculus
RESERVED

SEQ ID NO: 19 - protein, 8 aa, Mus musculus
RESERVED

SEQ ID NO: 20 - DNA, 321 bp, Mus musculus
  misc_feature: SC27.1 Light Chain Variable Region
gacatccagatgacacaatcttcatcctccttttctgtatctctaggagacagagtcaccattacttgcaaggcaagtgaagacatatataatcggttagcctggtatcagcagaaaccaggaaatgctcccaggctcttaatatctggtgcaaccagtttggaaactgggactccttcaagattcagtggcagtggatctggaaaggattacactctcagtattaccagtcttcggactgaagatgctgctacttattactgtcaacaatattggagtactccactcacgttcggtactgggaccaagctggagctgaaa

SEQ ID NO: 21 - protein, 107 aa, Mus musculus
  MISC_FEATURE: SC27.1 Light Chain Variable Region
DIQMTQSSSSFSVSLGDRVTITCKASEDIYNRLAWYQQKPGNAPRLLISGATSLETGTPSRFSGSGSGKDYTLSITSLRTEDAATYYCQQYWSTPLTFGTGTKLELK

SEQ ID NO: 22 - DNA, 357 bp, Mus musculus
  misc_feature: SC27.1 Heavy Chain Variable Region
gaggtccagctgcaagagtctagacctgagctggtgaagcctggggcttcagtgaagatatcctgcaagacttctggatacacattcactgaatacaccttgcactgggtgaagcagagtcatggaaagagccttgagtggattggaggtattaatcctaacaatggtgatactatctacaaccagaaattcaagggcaaggccacattgactgtagacaagtcctccagcacagcctacatggagctccgcagcctgacatctgaatattctgcagtctattactgtgcaagaagggcgattacggtctatgctatggactactggggtcaaggtacctcagtcaccgtctcctca

SEQ ID NO: 23 - protein, 119 aa, Mus musculus
  MISC_FEATURE: SC27.1 Heavy Chain Variable Region
EVQLQESRPELVKPGASVKISCKTSGYTFTEYTLHWVKQSHGKSLEWIGGINPNNGDTIYNQKFKGKATLTVDKSSSTAYMELRSLTSEYSAVYYCARRAITVYAMDYWGQGTSVTVSS

SEQ ID NO: 24 - DNA, 333 bp, Mus musculus
  misc_feature: SC27.22 Light Chain Variable Region
gacattgtgctgacacagtctcctgcttccttagctgtatctctggggcagagggccaccatctcatgcagggccagccagactgtcagtacatctagctatagttatatgcactggttccaacagaaaccaggacagccacccaaactcctcatcaagtttgcatccaacgtagaatctggggtccctgccagattcagtggcagtgggtctgggacagacttcaccctcaacatccatcctgtggaggaggaggatatttcaacatattactgtcagcacagttgggagattccgtggacgttcggtggaggcaccaagctggaaatcaaa

SEQ ID NO: 25 - protein, 111 aa, Mus musculus
  MISC_FEATURE: SC27.22 Light Chain Variable Region
DIVLTQSPASLAVSLGQRATISCRASQTVSTSSYSYMHWFQQKPGQPPKLLIKFASNVESGVPARFSGSGSGTDFTLNIHPVEEEDISTYYCQHSWEIPWTFGGGTKLEIK

SEQ ID NO: 26 - DNA, 366 bp, Mus musculus
  misc_feature: SC27.22 Heavy Chain Variable Region
caggtccaactgcagcagcctggggctgagctggtgaggcctggagcttcagtgaagctgtcctgcaaggcttctggctacaccttcaccagctactggatgaactgggtgaagcagaggcctggacaaggccttgaatggattgccatgattcatccttccgatagtgaaattaggttaaatcagaagttcaaggacaaggccacattgactgtagacagatcctccagcacagcctacatgcaactcagcagcccgacatctgaggactctgcggtctattactgtgcaagaattgatagttattatggttacctgttttactttgactactggggccaaggcaccactctcacagtctcctca

SEQ ID NO: 27 - protein, 122 aa, Mus musculus
  MISC_FEATURE: SC27.22 Heavy Chain Variable Region
QVQLQQPGAELVRPGASVKLSCKASGYTFTSYWMNWVKQRPGQGLEWIAMIHPSDSEIRLNQKFKDKATLTVDRSSSTAYMQLSSPTSEDSAVYYCARIDSYYGYLFYFDYWGQGTTLTVSS

SEQ ID NO: 28 - DNA, 324 bp, Mus musculus
  misc_feature: SC27.103 Light Chain Variable Region
caaattgttctcacccagtctccagcaatcatgtctgcatctctaggggaacgggtcaccatgacctgcactgccagctcaagtgtaagttccagttacttgcactggtaccagcagaagccaggatcctcccccacactctggatttataggacatccgacctggcttctggagtcccagctcgcttcagtggcagtggatctgggacctcttactctctcacaatcagcagcatggaggctgaagatgctgccacttattactgccaccagtatcatcgttccccgtggacgttcggtggaggcaccaggctggaaatcaaa

SEQ ID NO: 29 - protein, 108 aa, Mus musculus
  MISC_FEATURE: SC27.103 Light Chain Variable Region
QIVLTQSPAIMSASLGERVTMTCTASSSVSSSYLHWYQQKPGSSPTLWIYRTSDLASGVPARFSGSGSGTSYSLTISSMEAEDAATYYCHQYHRSPWTFGGGTRLEIK

SEQ ID NO: 30 - DNA, 354 bp, Mus musculus
  misc_feature: SC27.103 Heavy Chain Variable Region
gaggtccacctgcaacagtctggacctgagctagtgaagcctggaggttcaatgaagatatcctgcaaggcttctggttactcattcactggctacaccatgaactgggtgaagcagagccatggaaagaaccttgagtggattggactttttaatccttacaatggtggtactagttataaccagaagttcaagggcaaggccacattaactgtagacaagtcatccagcacagcctacatggagctcctcagtctgacatctgaggactctgcagtctattactgtgcaagatgctataggtacgacggtcttgactactggggccaaggcaccactctcacagtctcctca

SEQ ID NO: 31 - protein, 118 aa, Mus musculus
  MISC_FEATURE: SC27.103 Heavy Chain Variable Region
EVHLQQSGPELVKPGGSMKISCKASGYSFTGYTMNWVKQSHGKNLEWIGLFNPYNGGTSYNQKFKGKATLTVDKSSSTAYMELLSLTSEDSAVYYCARCYRYDGLDYWGQGTTLTVSS

SEQ ID NO: 32 - DNA, 321 bp, Mus musculus
  misc_feature: SC27.104 Light Chain Variable Region
gacatccagatgacacaatcttcatcctccttttctgtatctctaggagacagagtcaccattacttgcaaggcaagtgaggacatatataatcggttagcctggtatcagcagaaaccaggaaatgctcccaggctcttaatatctggtgcaaccagtttggaaactggggttccttcaagattcagtggcagtggatctggaaaggattacactctcagcattaccagtcttcagactgaagatgttgctacttattactgtcaacagtattggagtaatcctccgacgttcggtggaggcaccaagctggaaatcaaa

SEQ ID NO: 33 - protein, 107 aa, Mus musculus
  MISC_FEATURE: SC27.104 Light Chain Variable Region
DIQMTQSSSSFSVSLGDRVTITCKASEDIYNRLAWYQQKPGNAPRLLISGATSLETGVPSRFSGSGSGKDYTLSITSLQTEDVATYYCQQYWSNPPTFGGGTKLEIK

SEQ ID NO: 34 - DNA, 354 bp, Mus musculus
  misc_feature: SC27.104 Heavy Chain Variable Region
gaggtccagctgcaacagtctggacctgagctggtgaagcctggggcttcagtgaagatatcctgcaagacttctggatacacattcactgaatacaccgtgcactgggtgaagcagagccatggaaagagccttgagtggattggaggtgtttatcctaagaatggtgatactacctacaaccagaagttcaggggcaaggccacattgactgtagacaagtcctccaacacagcctatatggaactccgcagcctgacatctgaggattctgcagtctattactgtacaggaaaggatgggtacgacgggtttgcttactggggccaagggactctggtcactgtctctgca

SEQ ID NO: 35 - protein, 118 aa, Mus musculus
  MISC_FEATURE: SC27.104 Heavy Chain Variable Region
EVQLQQSGPELVKPGASVKISCKTSGYTFTEYTVHWVKQSHGKSLEWIGGVYPKNGDTTYNQKFRGKATLTVDKSSNTAYMELRSLTSEDSAVYYCTGKDGYDGFAYWGQGTLVTVSA

SEQ ID NO: 36 - DNA, 318 bp, Mus musculus
  misc_feature: SC27.105 Light Chain Variable Region
gatgttcaaatgacccagtctccatcctccctgtctgcatctttgggagagagagtctccctgacttgccaggcaagtcagagtgttagcaataatttaaactggtatcagcaaacaccagggaaagctcctaggctcttgatctatggtgcaagcaaattggaagatggggtctcttcaaggttcagtggcactggatatgggacagatttcactttcaccatcagcagcctggaggaagaagatgtggcaacttatttttgtctacagcataggtatctgtggacgttcggtggaggcaccaagctggaaatcaaa

SEQ ID NO: 37 - protein, 106 aa, Mus musculus
  MISC_FEATURE: SC27.105 Light Chain Variable Region
DVQMTQSPSSLSASLGERVSLTCQASQSVSNNLNWYQQTPGKAPRLLIYGASKLEDGVSSRFSGTGYGTDFTFTISSLEEEDVATYFCLQHRYLWTFGGGTKLEIK

SEQ ID NO: 38 - DNA, 369 bp, Mus musculus
  misc_feature: SC27.105 Heavy Chain Variable Region
gaggtccagctgcagcagtctggacctgagttggtgaagcctggggcttcagtgaagatatcctgcaaggcttctggttactcattcactggctactacatgaactgggtgaagcaaagtcctgaaaagagccttgagtggattggagagattaatcctagcactggtagtactacttacaaccagaagttcaaggccaaggccacattgactgtagacaaatcctccagcacagcctacatgcagctcaagagcctgacatctgaggactctgcagtctattactgtgcaagaagggattattactacggtagtggtttctatgctatggactactggggtcaaggaacctcagtcaccgtctcctca

SEQ ID NO: 39 - protein, 123 aa, Mus musculus
  MISC_FEATURE: SC27.105 Heavy Chain Variable Region
EVQLQQSGPELVKPGASVKISCKASGYSFTGYYMNWVKQSPEKSLEWIGEINPSTGSTTYNQKFKAKATLTVDKSSSTAYMQLKSLTSEDSAVYYCARRDYYYGSGFYAMDYWGQGTSVTVSS

SEQ ID NO: 40 - DNA, 321 bp, Mus musculus
  misc_feature: SC27.106 Light Chain Variable Region
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatgtggtgcaaccagtttggaaactggggttccttcaagattcagtggcagtggatctggaaaggattacactctcagcattaccagtcttcagactgaagatgttgctacttattactgtcaacagtattggagtactccgctcacgttcggtgctgggaccaaactggagctgaaa

SEQ ID NO: 41 - protein, 107 aa, Mus musculus
  MISC_FEATURE: SC27.106 Light Chain Variable Region
DIQMTQSSSSFSVSLGDRVTITCKASEDIYNRLAWYQQKPGNAPRLLICGATSLETGVPSRFSGSGSGKDYTLSITSLQTEDVATYYCQQYWSTPLTFGAGTKLELK

SEQ ID NO: 42 - DNA, 357 bp, Mus musculus
  misc_feature: SC27.106 Heavy Chain Variable Region
gaggtccagctgcaacagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtggtactaactacaaccagaagttcaagggcaaggccacattgactgttgacaagtcctccagcacagcctacatggagctccgcagcctgacatctgaggattctgcagtctattactgtgcaagaaggcttattacttactatgctatggactactggggtcaaggaacctcagtcaccgtctcctca

SEQ ID NO: 43 - protein, 119 aa, Mus musculus
  MISC_FEATURE: SC27.106 Heavy Chain Variable Region
EVQLQQSGPELVKPGASVKISCKTSGYTFTEYTMHWVKQSHGKSLEWIGGINPNNGGTNYNQKFKGKATLTVDKSSSTAYMELRSLTSEDSAVYYCARRLITYYAMDYWGQGTSVTVSS

SEQ ID NO: 44 - DNA, 324 bp, Mus musculus
  misc_feature: SC27.108 Light Chain Variable Region
gaaattgtgctcacccagtctccagcactcatggctgcatctccaggggagaaggtcaccatcacctgcagtgtcagctcaagtataagttccagcaacttgcactggtaccagcagaagtcaggaacctcccccaaactctggatttatggcacatccaacctggcttctggagtccctgttcgcttcagtggcagtggatctgggacctcttattctctcacaatcagcaacatggaggctgaagatgctgccacttattactgtcaacagtggagtagttacccacacacgttcggaggggggaccaagctggaaataaaa

SEQ ID NO: 45 - protein, 108 aa, Mus musculus
  MISC_FEATURE: SC27.108 Light Chain Variable Region
EIVLTQSPALMAASPGEKVTITCSVSSSISSSNLHWYQQKSGTSPKLWIYGTSNLASGVPVRFSGSGSGTSYSLTISNMEAEDAATYYCQQWSSYPHTFGGGTKLEIK

SEQ ID NO: 46 - DNA, 378 bp, Mus musculus
  misc_feature: SC27.108 Heavy Chain Variable Region
caggtccaaatgcagcagtctggagctgagctggtaaggcctgggacttcagtgaaggtgtcctgcaaggcttctggatacgccttcactaattacttgatagagtgggtaaagcagaggcctggacagggccttgagtggattggactgattaatcctggaagtggtggtactaattacaatgagaagttcaagggcaaggcaacactgactgcagacaaatcctccaccactgcctacatgcagctcagcagcctgacatctgatgactctgcggtttatttctgtgcaagacggtcccctctagggagttggatctactatgcttacgacggtgttgcttactggggccaagggactctggtcactgtctctgca

SEQ ID NO: 47 - protein, 126 aa, Mus musculus
  MISC_FEATURE: SC27.108 Heavy Chain Variable Region
QVQMQQSGAELVRPGTSVKVSCKASGYAFTNYLIEWVKQRPGQGLEWIGLINPGSGGTNYNEKFKGKATLTADKSSTTAYMQLSSLTSDDSAVYFCARRSPLGSWIYYAYDGVAYWGQGTLVTVSA

SEQ ID NO: 48 - DNA, 321 bp, Mus musculus
  misc_feature: SC27.201 Light Chain Variable Region
gacatccagatgacacaatcttcatcctccttttctgtctctctgggagacagagtcactattacttgcaaggcaagtgaggacatctataatcggttagcctggtatcaacagaaaccaggaaatgctcctaggctcttaatatctggtgcaaccagtttggaagctggggttccttcaggattcagtggcagtggatctggaaaggattacactctcagcattaccagtcttcagactgaagatgttgctacttattactgtcaacagtattggagtactcctccgacgttcggtggaggcaccaagctggaactcaag

SEQ ID NO: 49 - protein, 107 aa, Mus musculus
  MISC_FEATURE: SC27.201 Light Chain Variable Region
DIQMTQSSSSFSVSLGDRVTITCKASEDIYNRLAWYQQKPGNAPRLLISGATSLEAGVPSGFSGSGSGKDYTLSITSLQTEDVATYYCQQYWSTPPTFGGGTKLELK

SEQ ID NO: 50 - DNA, 360 bp, Mus musculus
  misc_feature: SC27.201 Heavy Chain Variable Region
gaggtccagctgcaacagtctggacctgaactggtgaagcctggggcttcagtgaagatatcctgcaagacttctggatacacattcactgaaaacatcagacactgggtgaagcagagccgaggaaagagccttgagtggattggtactattaatcctaataatggtgagactaggtacaatcagaagttcaagggcaaggccacattgactgtagacaagtcctccagcacagcctacatggagctccgcagcctgacatctgaggattctgcagtctattactgtacaagggggattacaaagtccccttatggtatggactactggggtcaaggaacctcaatcaccgtctcctca

SEQ ID NO: 51 - protein, 120 aa, Mus musculus
  MISC_FEATURE: SC27.201 Heavy Chain Variable Region
EVQLQQSGPELVKPGASVKISCKTSGYTFTENIRHWVKQSRGKSLEWIGTINPNNGETRYNQKFKGKATLTVDKSSSTAYMELRSLTSEDSAVYYCTRGITKSPYGMDYWGQGTSITVSS

SEQ ID NO: 52 - DNA, 321 bp, Mus musculus
  misc_feature: SC27.203 Light Chain Variable Region
gacatccagatgacacaatcttcatcctccttttctgtatctctaggagacagagtcaccatcacttgcaaggcaagtgaggacatatataatcggttagcctggtatcagcagaatccaggaaatactcctaggctcttaatgtctggtgcaaccagtttggaaactggggttccttcaagattcagtggcagtggatctggaaaggattacactctcagcattaccagtcttcagattgaagatgtttctacttattactgtcaacaatattggagtactcctccgacgttcggtggaggcaccaggctggaaatcaaa

SEQ ID NO: 53 - protein, 107 aa, Mus musculus
  MISC_FEATURE: SC27.203 Light Chain Variable Region
DIQMTQSSSSFSVSLGDRVTITCKASEDIYNRLAWYQQNPGNTPRLLMSGATSLETGVPSRFSGSGSGKDYTLSITSLQIEDVSTYYCQQYWSTPPTFGGGTRLEIK

SEQ ID NO: 54 - DNA, 360 bp, Mus musculus
  misc_feature: SC27.203 Heavy Chain Variable Region
gaggtccagctgcaacagtctggacctgagctggtgaagcctggggcttcagtgaagatatcctgcaagacttctggatacacattcactgaaaacatcatacactgggtgaagcagagccatggaaagagccttgagtggattggaggtattaatcctatcaatggtggtactagctacaaccagaagttcaagggcaaggccacattgactgtagacaagtcctccagcacagcctacatggagctccgtagcctgacatctgaggattctgcagtctattactgtgcaagggggattactacgtccccttatgctatggactactggggtcaaggaacctcagtcaccgtctcctca

SEQ ID NO: 55 - protein, 120 aa, Mus musculus
  MISC_FEATURE: SC27.203 Heavy Chain Variable Region
EVQLQQSGPELVKPGASVKISCKTSGYTFTENIIHWVKQSHGKSLEWIGGINPINGGTSYNQKFKGKATLTVDKSSSTAYMELRSLTSEDSAVYYCARGITTSPYAMDYWGQGTSVTVSS

SEQ ID NO: 56 - DNA, 321 bp, Mus musculus
  misc_feature: SC27.204 Light Chain Variable Region
gacattgtgatgacccagtctcaaaaattcatgtccacatcagtaggagacagggtcagcgtcgcctgcaaggccggtcagaatgtgggtactagtgtagcctggtatcaacagaaaccaggacattctcctaaatcactgatttactcggcatcctaccggtacagtggagtccctaatcgcttcacaggcagtggatctgggacagatttcactctcaccatcagcaatgtgcagtctgaagacttggcagactatttctgtcagcaatatatcacctatccgtacacgttcggaggggggaccaagctggaaataata

SEQ ID NO: 57 - protein, 107 aa, Mus musculus
  MISC_FEATURE: SC27.204 Light Chain Variable Region
DIVMTQSQKFMSTSVGDRVSVACKAGQNVGTSVAWYQQKPGHSPKSLIYSASYRYSGVPNRFTGSGSGTDFTLTISNVQSEDLADYFCQQYITYPYTFGGGTKLEII

SEQ ID NO: 58 - DNA, 336 bp, Mus musculus
  misc_feature: SC27.204 Heavy Chain Variable Region
gaggtgaaggttctcgagtctggaggtggcctggtgcagcctggaggatccctgaaactctcctgtgcagcctcaggattcgattttagtagatactggatgagttgggtccggcaggctccagggaaaggcctagaatggattggagaaattaatccagatagcagtacgataaactatacgccatctctaaaggctaaattcatcatctccagagacaacgccaaaaatacgctgtacctgcaaatgagcaaagtgagatctgaggacacagccctttattactgtacaggaccagcttactggggccaagggactctggtcactgtctctgca

SEQ ID NO: 59 - protein, 112 aa, Mus musculus
  MISC_FEATURE: SC27.204 Heavy Chain Variable Region
EVKVLESGGGLVQPGGSLKLSCAASGFDFSRYWMSWVRQAPGKGLEWIGEINPDSSTINYTPSLKAKFIISRDNAKNTLYLQMSKVRSEDTALYYCTGPAYWGQGTLVTVSA

SEQ ID NO: 60 - DNA, 321 bp, Artificial Sequence
  hSC27.1 Light Chain Variable Region
gacatccagatgacccagtctccatcttccgtgtctgcatctgtaggagacagagtcaccatcacttgtaaggcgagtgaggatatttacaaccggttagcctggtatcagcagaaaccagggaaagcccctaagctcctgatctatggtgcaaccagtttggaaactggggtcccatcaaggttcagcggcagtggatctgggacagattacactctcaccatcagcagcctgcagcctgaagattttgcaacttactattgtcaacagtattggagtactccactcacgttcggtcaagggaccaagctggagattaaa

SEQ ID NO: 61 - protein, 107 aa, Artificial Sequence
  hSC27.1 Light Chain Variable Region
DIQMTQSPSSVSASVGDRVTITCKASEDIYNRLAWYQQKPGKAPKLLIYGATSLETGVPSRFSGSGSGTDYTLTISSLQPEDFATYYCQQYWSTPLTFGQGTKLEIK

SEQ ID NO: 62 - DNA, 357 bp, Artificial Sequence
  hSC27.1 Heavy Chain Variable Region
caggtccagcttgtgcagtctggggctgaggtgaagaagcctggggcctcagtgaaggtttcctgcaaggcttctggatacaccttcactgagtatactctgcattgggtgcgccaggcccccggacaaaggcttgagtggatgggagggatcaaccctaacaatggtgacacaatatataaccagaagttcaagggcagagtcaccattaccagggacacatccgcgagcacagcctacatggagctgagcagcctgagatctgaagacacggctgtgtattactgtgcgagaagggcgattacggtctatgctatggactactggggtcaaggtaccctagtcaccgtctcgagc

SEQ ID NO: 63 - protein, 119 aa, Artificial Sequence
  hSC27.1 Heavy Chain Variable Region
QVQLVQSGAEVKKPGASVKVSCKASGYTFTEYTLHWVRQAPGQRLEWMGGINPNNGDTIYNQKFKGRVTITRDTSASTAYMELSSLRSEDTAVYYCARRAITVYAMDYWGQGTLVTVSS

SEQ ID NO: 64 - DNA, 333 bp, Artificial Sequence
  hSC27.22 Light Chain Variable Region
gacattgtcatgacccagtcccctgacagtttggccgttagcttgggggagcgtgccaccatcaactgtagggctagtcaaactgtttctacatcctcctactcttacatgcattggtatcagcagaaacctggtcagcctccaaaactgctgatttatttcgcatctaacgtcgagtccggagttcctgaccggttcagcggatcaggaagcggtacagattttacacttaccatctcatctctgcaagcagaagatgtggccgtgtactattgtcagcattcctgggagatcccctggaccttcgggcagggaaccaagctcgagattaaa

SEQ ID NO: 65 - protein, 111 aa, Artificial Sequence
  hSC27.22 Light Chain Variable Region
DIVMTQSPDSLAVSLGERATINCRASQTVSTSSYSYMHWYQQKPGQPPKLLIYFASNVESGVPDRFSGSGSGTDFTLTISSLQAEDVAVYYCQHSWEIPWTFGQGTKLEIK

SEQ ID NO: 66 - DNA, 366 bp, Artificial Sequence
  hSC27.22 Heavy Chain Variable Region
caggtgcagttggtgcagagcggcgccgaagtcaagaaaccaggagcttctgtcaaagtctcctgtaaagcctccggatataccttcaccagctactggatgaattgggtaagacaggcccccggacagaggcttgagtggatgggaatgatccatccctctgacagcgagattcggctcaaccagaagtttaaagaccgagtgactatcacacgcgataccagtgctagcacagcctacatggagttgagttctcttcgtagcgaggacactgccgtgtattattgcgcccgcatcgactcatattatggttatctgttctacttcgactattggggccaggggaccaccgtgactgtgtcttcc

SEQ ID NO: 67 - protein, 122 aa, Artificial Sequence
  hSC27.22 Heavy Chain Variable Region
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSS

SEQ ID NO: 68 - DNA, 324 bp, Artificial Sequence
  hSC27.108 Light Chain Variable Region
gaaatcgtgcttacacaatcccctgccactctgagcctttctccaggcgagcgagcaaccctttcctgcagtgtttcctcttcaatcagttccagcaatttgcactggtaccagcagaagcctggtcaggcaccccgattgttgatctatggcacatctaacctggccagcggcatccctgctcggttcagtggatctggctccggaacagatttcactctcactatcagctcccttgagcctgaagattttgccgtgtactactgtcagcaatggagttcctacccccacacctttggcggcgggacaaaggtcgagataaaa

SEQ ID NO: 69 - protein, 108 aa, Artificial Sequence
  hSC27.108 Light Chain Variable Region
EIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIK

SEQ ID NO: 70 - DNA, 378 bp, Artificial Sequence
  hSC27.108 Heavy Chain Variable Region
caggtacagctggtccagtccggcgctgaggttaagaagcccggtgcctccgtgaaggtatcttgtaaggcctcaggttacacctttacaaattatctgatcgaatgggtgagacaggccccaggtcagggtctggaatggatgggactcatcaaccctgggagtggcgggaccaactacaacgaaaagtttaaggggagagtgacaatgaccacagataccagtacctccaccgcatatatggagctgcgaagcttgaggtccgatgacactgctgtgtactattgcgcccgtagaagcccactcgggtcttggatctattacgcatacgatggtgtggcctattggggccagggcaccctggtgacagtcagctcc

SEQ ID NO: 71 - protein, 126 aa, Artificial Sequence
  hSC27.108 Heavy Chain Variable Region
QVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSS

SEQ ID NO: 72 - DNA, 321 bp, Artificial Sequence
  hSC27.204 Light Chain Variable Region
gacatccagatgacccagtccccctccagcctgtctgcttccgtgggcgacagagtgaccatcacatgcaaggccggccagaacgtgggcacctctgtggcctggttccagcagaagcctggcaaggcccccaagtccctgatctactccgcctcctacagatactccggcgtgccctccagattctccggctctggctctggcaccgactttaccctgaccatcagctccctgcagcccgaggacttcgccacctactactgccagcagtacatcacctacccctacaccttcggcggaggcaccaaggtggaaatcaag

SEQ ID NO: 73 - protein, 107 aa, Artificial Sequence
  hSC27.204 Light Chain Variable Region
DIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIK

SEQ ID NO: 74 - DNA, 336 bp, Artificial Sequence
  hSC27.204 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaactacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 75 - protein, 112 aa, Artificial Sequence
  hSC27.204 Heavy Chain Variable Region
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCTGPAYWGQGTLVTVSS

SEQ ID NO: 76 - DNA, 324 bp, Artificial Sequence
  hSC27.108v1 Light Chain Variable Region
gaaatcgtgctgactcagtctcccgatttccagtccgtcacacccaaggagaaagtcaccatcacttgttctgtctcctcaagcatctcttctagtaacctgcactggtatcagcagaagcctgatcagtcccctaagctttggatatacggcacctcaaacctcgcctccggagttccttcaaggttttcaggtagtggatctggaaccgatttcacccttacaataaacagtcttgaggccgaggacgccgccacttactactgccagcagtggagttcttacccccacacatttgggggcggcaccaaagtggagatcaaa

SEQ ID NO: 77 - protein, 108 aa, Artificial Sequence
  hSC27.108v1 Light Chain Variable Region
EIVLTQSPDFQSVTPKEKVTITCSVSSSISSSNLHWYQQKPDQSPKLWIYGTSNLASGVPSRFSGSGSGTDFTLTINSLEAEDAATYYCQQWSSYPHTFGGGTKVEIK

SEQ ID NO: 78 - DNA, 366 bp, Artificial Sequence
  hSC27.22-VH1-8 Heavy Chain Variable Region
caggtgcagctggtgcagtctggcgccgaagtgaagaaacctggcgcctccgtgaaggtgtcctgcaaggcctccggctacacctttaccagctactggatgaactgggtgcgacaggctaccggccagggcctggaatggatgggcatgatccacccctccgactccgagatccggctgaaccagaaattcaaggacagagtgaccatgacccggaacacctccatctccaccgcctacatggaactgtcctccctgcggagcgaggacaccgccgtgtactactgcgcccggatcgactcctactacggctacctgttctacttcgactactggggccagggcaccaccgtgaccgtgtcatct

SEQ ID NO: 79 - protein, 122 aa, Artificial Sequence
  hSC27.22-VH1-8 Heavy Chain Variable Region
QVQLVQSGAEVKKPGASVKVSCKASGYTFTSYWMNWVRQATGQGLEWMGMIHPSDSEIRLNQKFKDRVTMTRNTSISTAYMELSSLRSEDTAVYYCARIDSYYGYLFYFDYWGQGTTVTVSS

SEQ ID NO: 80 - DNA, 366 bp, Artificial Sequence
  hSC27.22-VH1-46 Heavy Chain Variable Region
caggtgcagctggtgcagtctggcgccgaagtgaagaaacctggcgcctccgtgaaggtgtcctgcaaggcctccggctacacctttaccagctactggatgaactgggtgcgacaggcccctggacagggcctggaatggatgggcatgatccacccctccgactccgagatccggctgaaccagaaattcaaggaccgcgtgaccatgaccagagacacctccaccagcaccgtgtacatggaactgtcctccctgcggagcgaggacaccgccgtgtactactgcgcccggatcgactcctactacggctacctgttctacttcgactactggggccagggcaccaccgtgaccgtgtcatct

SEQ ID NO: 81 - protein, 122 aa, Artificial Sequence
  hSC27.22-VH1-46 Heavy Chain Variable Region
QVQLVQSGAEVKKPGASVKVSCKASGYTFTSYWMNWVRQAPGQGLEWMGMIHPSDSEIRLNQKFKDRVTMTRDTSTSTVYMELSSLRSEDTAVYYCARIDSYYGYLFYFDYWGQGTTVTVSS

SEQ ID NO: 82 - DNA, 366 bp, Artificial Sequence
  hSC27.22-VH1-69 Heavy Chain Variable Region
caggtgcagctggtgcagtctggcgccgaagtgaagaaacccggctcctccgtgaaggtgtcctgcaaggcttccggcggcaccttctccagctactggatgaactgggtgcgacaggcccctggacagggcctggaatggatgggcatgatccacccctccgactccgagatccggctgaaccagaaattcaaggacagagtgaccatcaccgccgacgagtccacctccaccgcctacatggaactgtcctccctgcggagcgaggacaccgccgtgtactactgcgcccggatcgactcctactacggctacctgttctacttcgactactggggccagggcaccaccgtgaccgtgtcatct

SEQ ID NO: 83 - protein, 122 aa, Artificial Sequence
  hSC27.22-VH1-69 Heavy Chain Variable Region
QVQLVQSGAEVKKPGSSVKVSCKASGGTFSSYWMNWVRQAPGQGLEWMGMIHPSDSEIRLNQKFKDRVTITADESTSTAYMELSSLRSEDTAVYYCARIDSYYGYLFYFDYWGQGTTVTVSS

SEQ ID NO: 84 - DNA, 336 bp, Artificial Sequence
  hSC27.204v1 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 85 - protein, 112 aa, Artificial Sequence
  hSC27.204v1 Heavy Chain Variable Region
EVQLLESGGGLVQPGGSLRLSCAASGFTFSRYWMSWVRQAPGKGLEWVSEINPDSSTIKYTPSLKARFTISRDNSKNTLYLQMNSLRAEDTAVYYCTGPAYWGQGTLVTVSS

SEQ ID NO: 86 - DNA, 336 bp, Artificial Sequence
  hSC27.204v2 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 87 - protein, 112 aa, Artificial Sequence
  hSC27.204v2 Heavy Chain Variable Region
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSS

SEQ ID NO: 88 - DNA, 336 bp, Artificial Sequence
  hSC27.204v3 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaactacaaccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 89 - protein, 112 aa, Artificial Sequence
  hSC27.204v3 Heavy Chain Variable Region
EVQLLESGGGLVQPGGSLRLSCAASGFTFSRYWMSWVRQAPGKGLEWVSEINPDSSTINYNPSLKARFTISRDNSKNTLYLQMNSLRAEDTAVYYCTGPAYWGQGTLVTVSS

SEQ ID NO: 90 - DNA, 336 bp, Artificial Sequence
  hSC27.204v4 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaactacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 91 - protein, 112 aa, Artificial Sequence
  hSC27.204v4 Heavy Chain Variable Region
EVQLLESGGGLVQPGGSLRLSCAASGFDFSRYWMSWVRQAPGKGLEWVSEINPDSSTINYTPSLKARFTISRDNSKNTLYLQMNSLRAEDTAVYYCTGPAYWGQGTLVTVSS

SEQ ID NO: 92 - DNA, 336 bp, Artificial Sequence
  hSC27.204v5 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 93 - protein, 112 aa, Artificial Sequence
  hSC27.204v5 Heavy Chain Variable Region
EVQLLESGGGLVQPGGSLRLSCAASGFDFSRYWMSWVRQAPGKGLEWVSEINPDSSTIKYTPSLKARFTISRDNSKNTLYLQMNSLRAEDTAVYYCTGPAYWGQGTLVTVSS

SEQ ID NO: 94 - DNA, 336 bp, Artificial Sequence
  hSC27.204v6 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 95 - protein, 112 aa, Artificial Sequence
  hSC27.204v6 Heavy Chain Variable Region
EVQLLESGGGLVQPGGSLRLSCAASGFDFSRYWMSWVRQAPGKGLEWVSEINPDSSTIQYTPSLKARFTISRDNSKNTLYLQMNSLRAEDTAVYYCTGPAYWGQGTLVTVSS

SEQ ID NO: 96 - DNA, 336 bp, Artificial Sequence
  hSC27.204v7 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaactacaaccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 97 - protein, 112 aa, Artificial Sequence
  hSC27.204v7 Heavy Chain Variable Region
EVQLLESGGGLVQPGGSLRLSCAASGFDFSRYWMSWVRQAPGKGLEWVSEINPDSSTINYNPSLKARFTISRDNSKNTLYLQMNSLRAEDTAVYYCTGPAYWGQGTLVTVSS

SEQ ID NO: 98 - DNA, 336 bp, Artificial Sequence
  hSC27.204v8 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaactacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 99 - protein, 112 aa, Artificial Sequence
  hSC27.204v8 Heavy Chain Variable Region
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCAGPAYWGQGTLVTVSS

SEQ ID NO: 100 - DNA, 336 bp, Artificial Sequence
  hSC27.204v9 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaagtacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 101 - protein, 112 aa, Artificial Sequence
  hSC27.204v9 Heavy Chain Variable Region
EVQLLESGGGLVQPGGSLRLSCAASGFTFSRYWMSWVRQAPGKGLEWVSEINPDSSTIKYTPSLKARFTISRDNSKNTLYLQMNSLRAEDTAVYYCAGPAYWGQGTLVTVSS

SEQ ID NO: 102 - DNA, 336 bp, Artificial Sequence
  hSC27.204v10 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 103 - protein, 112 aa, Artificial Sequence
  hSC27.204v10 Heavy Chain Variable Region
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCAGPAYWGQGTLVTVSS

SEQ ID NO: 104 - DNA, 336 bp, Artificial Sequence
  hSC27.204v11 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaactacaaccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 105 - protein, 112 aa, Artificial Sequence
  hSC27.204v11 Heavy Chain Variable Region
EVQLLESGGGLVQPGGSLRLSCAASGFTFSRYWMSWVRQAPGKGLEWVSEINPDSSTINYNPSLKARFTISRDNSKNTLYLQMNSLRAEDTAVYYCAGPAYWGQGTLVTVSS

SEQ ID NO: 106 - DNA, 336 bp, Artificial Sequence
  hSC27.204v12 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaactacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 107 - protein, 112 aa, Artificial Sequence
  hSC27.204v12 Heavy Chain Variable Region
EVQLLESGGGLVQPGGSLRLSCAASGFDFSRYWMSWVRQAPGKGLEWVSEINPDSSTINYTPSLKARFTISRDNSKNTLYLQMNSLRAEDTAVYYCAGPAYWGQGTLVTVSS

SEQ ID NO: 108 - DNA, 336 bp, Artificial Sequence
  hSC27.204v13 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaagtacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 109 - protein, 112 aa, Artificial Sequence
  hSC27.204v13  Heavy Chain Variable Region
EVQLLESGGGLVQPGGSLRLSCAASGFDFSRYWMSWVRQAPGKGLEWVSEINPDSSTIKYTPSLKARFTISRDNSKNTLYLQMNSLRAEDTAVYYCAGPAYWGQGTLVTVSS

SEQ ID NO: 110 - DNA, 336 bp, Artificial Sequence
  hSC27.204v14 Heavy Chain Variable Region Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 111 - protein, 112 aa, Artificial Sequence
  hSC27.204v14 Heavy Chain Variable Region
EVQLLESGGGLVQPGGSLRLSCAASGFDFSRYWMSWVRQAPGKGLEWVSEINPDSSTIQYTPSLKARFTISRDNSKNTLYLQMNSLRAEDTAVYYCAGPAYWGQGTLVTVSS

SEQ ID NO: 112 - DNA, 336 bp, Artificial Sequence
  hSC27.204v15 Heavy Chain Variable Region
gaagtgcagctgctggaatctggcggcggactggtgcagcctggcggatctctgagactgtcttgtgccgcctccggcttcgacttctcccggtactggatgtcctgggtgcgacaggctcctggcaagggcctggaatgggtgtccgagatcaaccccgactcctccaccatcaactacaaccccagcctgaaggcccggttcaccatctctcgggacaactccaagaacaccctgtacctgcagatgaactccctgcgggccgaggacaccgccgtgtactactgtgccggccctgcttattggggccagggcaccctcgtgaccgtgtcctct

SEQ ID NO: 113 - protein, 112 aa, Artificial Sequence
  hSC27.204v15 Heavy Chain Variable Region
EVQLLESGGGLVQPGGSLRLSCAASGFDFSRYWMSWVRQAPGKGLEWVSEINPDSSTINYNPSLKARFTISRDNSKNTLYLQMNSLRAEDTAVYYCAGPAYWGQGTLVTVSS

SEQ ID NO: 114 - protein, 214 aa, Artificial Sequence
  hSC27.1 Light Chain
DIQMTQSPSSVSASVGDRVTITCKASEDIYNRLAWYQQKPGKAPKLLIYGATSLETGVPSRFSGSGSGTDYTLTISSLQPEDFATYYCQQYWSTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 448 aa, Artificial Sequence
  hSC27.1 Heavy Chain
QVQLVQSGAEVKKPGASVKVSCKASGYTFTEYTLHWVRQAPGQRLEWMGGINPNNGDTIYNQKFKGRVTITRDTSASTAYMELSSLRSEDTAVYYCARRAITVYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 218 aa, Artificial Sequence
  hSC27.22 Light Chain
DIVMTQSPDSLAVSLGERATINCRASQTVSTSSYSYMHWYQQKPGQPPKLLIYFASNVESGVPDRFSGSGSGTDFTLTISSLQAEDVAVYYCQHSWEIPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 451 aa, Artificial Sequence
  hSC27.22 Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 215 aa, Artificial Sequence
  hSC27.108 Light Chain
EIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 455 aa, Artificial Sequence
  hSC27.108 Heavy Chain
QVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 214 aa, Artificial Sequence
  hSC27.204 Light Chain
DIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 441 aa, Artificial Sequence
  hSC27.204 Heavy Chain
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 451 aa, Artificial Sequence
  hSC27.22ss1 Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 451 aa, Artificial Sequence
  hSC27.22-VH1-8 Heavy Chain
QVQLVQSGAEVKKPGASVKVSCKASGYTFTSYWMNWVRQATGQGLEWMGMIHPSDSEIRLNQKFKDRVTMTRNTSISTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 451 aa, Artificial Sequence
  hSC27.22-VH1-46 Heavy Chain
QVQLVQSGAEVKKPGASVKVSCKASGYTFTSYWMNWVRQAPGQGLEWMGMIHPSDSEIRLNQKFKDRVTMTRDTSTSTVYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 451 aa, Artificial Sequence
  hSC27.22-VH1-69 Heavy Chain
QVQLVQSGAEVKKPGSSVKVSCKASGGTFSSYWMNWVRQAPGQGLEWMGMIHPSDSEIRLNQKFKDRVTITADESTSTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 447 aa, Artificial Sequence
  hSC27.22 IgG2 Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 127 - protein, 448 aa, Artificial Sequence
  hSC27.22 IgG4 R409K Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 128 - protein, 448 aa, Artificial Sequence
  hSC27.22 IgG4 S228P Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 129 - protein, 448 aa, Artificial Sequence
  hSC27.22 IgG4 S228P K370E R409K Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVEGFYPSDIAVEWESNGQPENNYKTTPPVLDSDGSFFLYSKLTVDKSRWQEGNVFSCSVMHEALHNHYTQKSLSLSLG

SEQ ID NO: 130 - protein, 448 aa, Artificial Sequence
  hSC27.22 IgG4 K370E Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVEGFYPSDIAVEWESNGQPENNYKTTPPVLDSDGSFFLYSRLTVDKSRWQEGNVFSCSVMHEALHNHYTQKSLSLSLG

SEQ ID NO: 131 - protein, 448 aa, Artificial Sequence
  hSC27.22 IgG4 S228P K370E Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVEGFYPSDIAVEWESNGQPENNYKTTPPVLDSDGSFFLYSRLTVDKSRWQEGNVFSCSVMHEALHNHYTQKSLSLSLG

SEQ ID NO: 132 - protein, 448 aa, Artificial Sequence
  hSC27.22 IgG4 C127S S228P Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 133 - protein, 448 aa, Artificial Sequence
  hSC27.22 IgG4 C127S K370E Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVEGFYPSDIAVEWESNGQPENNYKTTPPVLDSDGSFFLYSRLTVDKSRWQEGNVFSCSVMHEALHNHYTQKSLSLSLG

SEQ ID NO: 134 - protein, 448 aa, Artificial Sequence
  hSC27.22 IgG4 C127S S228P K370E Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVEGFYPSDIAVEWESNGQPENNYKTTPPVLDSDGSFFLYSRLTVDKSRWQEGNVFSCSVMHEALHNHYTQKSLSLSLG

SEQ ID NO: 135 - protein, 215 aa, Artificial Sequence
  hSC27.108v1 Heavy Chain
EIVLTQSPDFQSVTPKEKVTITCSVSSSISSSNLHWYQQKPDQSPKLWIYGTSNLASGVPSRFSGSGSGTDFTLTINSLEAEDAATYYCQQWSSYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 441 aa, Artificial Sequence
  hSC27.204v1 Heavy Chain
EVQLLESGGGLVQPGGSLRLSCAASGFTFSRYWMSWVRQAPGKGLEWVSEINPDSSTIKYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 441 aa, Artificial Sequence
  hSC27.204v2 Heavy Chain
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 441 aa, Artificial Sequence
  hSC27.204v3 Heavy Chain
EVQLLESGGGLVQPGGSLRLSCAASGFTFSRYWMSWVRQAPGKGLEWVSEINPDSSTINYNPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 441 aa, Artificial Sequence
  hSC27.204v4 Heavy Chain
EVQLLESGGGLVQPGGSLRLSCAASGFDFSRYWMSWVRQAPGKGLEWVSEINPDSSTINYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 441 aa, Artificial Sequence
  hSC27.204v5 Heavy Chain
EVQLLESGGGLVQPGGSLRLSCAASGFDFSRYWMSWVRQAPGKGLEWVSEINPDSSTIKYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 441 aa, Artificial Sequence
  hSC27.204v6 Heavy Chain
EVQLLESGGGLVQPGGSLRLSCAASGFDFSRYWMSWVRQAPGKGLEWVSEINPDSSTIQYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 441 aa, Artificial Sequence
  hSC27.204v7 Heavy Chain
EVQLLESGGGLVQPGGSLRLSCAASGFDFSRYWMSWVRQAPGKGLEWVSEINPDSSTINYNPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 441 aa, Artificial Sequence
  hSC27.204v8 Heavy Chain
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 441 aa, Artificial Sequence
  hSC27.204v9 Heavy Chain
EVQLLESGGGLVQPGGSLRLSCAASGFTFSRYWMSWVRQAPGKGLEWVSEINPDSSTIKYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 441 aa, Artificial Sequence
  hSC27.204v10 Heavy Chain
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 441 aa, Artificial Sequence
  hSC27.204v11 Heavy Chain
EVQLLESGGGLVQPGGSLRLSCAASGFTFSRYWMSWVRQAPGKGLEWVSEINPDSSTINYNPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 441 aa, Artificial Sequence
  hSC27.204v12 Heavy Chain
EVQLLESGGGLVQPGGSLRLSCAASGFDFSRYWMSWVRQAPGKGLEWVSEINPDSSTINYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 441 aa, Artificial Sequence
  hSC27.204v13 Heavy Chain
EVQLLESGGGLVQPGGSLRLSCAASGFDFSRYWMSWVRQAPGKGLEWVSEINPDSSTIKYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 441 aa, Artificial Sequence
  hSC27.204v14 Heavy Chain
EVQLLESGGGLVQPGGSLRLSCAASGFDFSRYWMSWVRQAPGKGLEWVSEINPDSSTIQYTPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 441 aa, Artificial Sequence
  hSC27.204v15 Heavy Chain
EVQLLESGGGLVQPGGSLRLSCAASGFDFSRYWMSWVRQAPGKGLEWVSEINPDSSTINYNPSLKARFTISRDNSKNTLYLQMNSLRAEDTAVYYCAGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 11 aa, Artificial Sequence
  hSC27.1 CDRL1
KASEDIYNRLA

SEQ ID NO: 152 - protein, 7 aa, Artificial Sequence
  hSC27.1 CDRL2
GATSLET

SEQ ID NO: 153 - protein, 9 aa, Artificial Sequence
  hSC27.1 CDRL3
QQYWSTPLT

SEQ ID NO: 154 - protein, 5 aa, Artificial Sequence
  hSC27.1 CDRH1
EYTLH

SEQ ID NO: 155 - protein, 17 aa, Artificial Sequence
  hSC27.1 CDRH2
GINPNNGDTIYNQKFKG

SEQ ID NO: 156 - protein, 10 aa, Artificial Sequence
  hSC27.1 CDRH3
RAITVYAMDY

SEQ ID NO: 157 - protein, 15 aa, Artificial Sequence
  hSC27.22 CDRL1
RASQTVSTSSYSYMH

SEQ ID NO: 158 - protein, 7 aa, Artificial Sequence
  hSC27.22 CDRL2
FASNVES

SEQ ID NO: 159 - protein, 9 aa, Artificial Sequence
  hSC27.22 CDRL3
QHSWEIPWT

SEQ ID NO: 160 - protein, 5 aa, Artificial Sequence
  hSC27.22 CDRH1
SYWMN

SEQ ID NO: 161 - protein, 17 aa, Artificial Sequence
  hSC27.22 CDRH2
MIHPSDSEIRLNQKFKD

SEQ ID NO: 162 - protein, 13 aa, Artificial Sequence
  hSC27.22 CDRH3
IDSYYGYLFYFDY

SEQ ID NO: 163 - protein, 12 aa, Artificial Sequence
  hSC27.108 CDRL1
SVSSSISSSNLH

SEQ ID NO: 164 - protein, 7 aa, Artificial Sequence
  hSC27.108 CDRL2
GTSNLAS

SEQ ID NO: 165 - protein, 9 aa, Artificial Sequence
  hSC27.108 CDRL3
QQWSSYPHT

SEQ ID NO: 166 - protein, 5 aa, Artificial Sequence
  hSC27.108 CDRH1
NYLIE

SEQ ID NO: 167 - protein, 17 aa, Artificial Sequence
  hSC27.108 CDRH2
LINPGSGGTNYNEKFKG

SEQ ID NO: 168 - protein, 17 aa, Artificial Sequence
  hSC27.108 CDRH3
RSPLGSWIYYAYDGVAY

SEQ ID NO: 169 - protein, 11 aa, Artificial Sequence
  hSC27.204 CDRL1
KAGQNVGTSVA

SEQ ID NO: 170 - protein, 7 aa, Artificial Sequence
  hSC27.204 CDRL2
SASYRYS

SEQ ID NO: 171 - protein, 9 aa, Artificial Sequence
  hSC27.204 CDRL3
QQYITYPYT

SEQ ID NO: 172 - protein, 5 aa, Artificial Sequence
  hSC27.204 CDRH1
RYWMS

SEQ ID NO: 173 - protein, 17 aa, Artificial Sequence
  hSC27.204 CDRH2
EINPDSSTINYTPSLKA

SEQ ID NO: 174 - protein, 3 aa, Artificial Sequence
  hSC27.204 CDRH3
PAY

SEQ ID NO: 175 - protein, 17 aa, Artificial Sequence
  hSC27.204v1, hSC27.204v5 and hSC27.405v13 CDRH2
EINPDSSTIKYTPSLKA

SEQ ID NO: 176 - protein, 17 aa, Artificial Sequence
  hSC27.204v2, hSC27.204v6 and hSC27.405v14 CDRH2
EINPDSSTIQYTPSLKA

SEQ ID NO: 177 - protein, 17 aa, Artificial Sequence
  hSC27.204v3, hSC27.204v7 and hSC27.405v15 CDRH2
EINPDSSTINYNPSLKA

SEQ ID NO: 178 - DNA, 366 bp, Artificial Sequence
  Codon optimized hSC27.22ss1 full length HC DNA
caagtgcagctcgtccagtccggtgccgaagtcaagaagccgggcgcatcagtgaaagtgtcgtgcaaagcctccgggtacaccttcacctcatactggatgaactgggtccgccaagccccgggacagagactggagtggatgggcatgattcacccatccgattccgagatccggctgaaccagaagttcaaggaccgcgtgaccatcacccgggacaccagcgccagcactgcctacatggaattgagctcgctgcggtccgaggataccgctgtgtactattgcgcgaggatcgactcctactacggctaccttttctacttcgactactggggacaagggacgaccgtgactgtgtcgagc